(19) United States
(12) Patent Application Publication
(10) Pub. No.: US 2023/0381377 A1
(43) Pub. Date: Nov. 30, 2023

(54) DIALDEHYDE STARCH CROSSLINKED SCAFFOLD COMPOSITIONS AND METHODS

(71) Applicant: Scripps Health, San Diego, CA (US)

(72) Inventors: Darryl D. D'LIMA, San Diego, CA (US); Clifford W. COLWELL, JR., San Diego, CA (US); Kwang Il LEE, San Diego, CA (US)

(21) Appl. No.: 18/248,319

(22) PCT Filed: Oct. 6, 2021
(86) PCT No.: PCT/US2021/053793

§ 371 Date: Apr. 7, 2023

Related U.S. Application Data
(60) Provisional application No. 63/089,504, filed on Oct. 8, 2020.

(51) Int. Cl. A61L 27/52; A61L 27/38; A61L 27/26; A61L 27/36; A61L 27/54; B33Y 70/00

(52) U.S. Cl. CPC A61L 27/52; A61L 27/3817; A61L 27/3834; A61L 27/26; A61L 27/3852; A61L 27/3633; A61L 27/54; A61L 27/3654; B33Y 70/00; A61L 2400/06; A61L 2430/24; A61L 2300/414; A61L 2430/34; A61L 2430/06

(57) ABSTRACT

Provided herein are compositions comprising collagen, dialdehyde starch, and at least one population of cells. Also, provided herein are methods of bioprinting and methods of producing cell-laden, three-dimensional scaffolds, comprising the compositions described herein.

### CROSS-REFERENCE

This application claims the benefit of U.S. Provisional Application No. 63/089,504, filed Oct. 8, 2020, which is incorporated herein by reference in its entity.

### BACKGROUND

Tissue substitutes made of biomimetic material and cells have drawn tremendous attention in the field of regenerative medicine as an optimal treatment for damaged human tissue. More recently, three dimensional (3D) bioprinting technology using bio-inks have emerged to provide spatially-controlled and personalized substitutes for individual tissues of a patient. However, existing tissue substitute materials lack many desired physical and chemical properties. These properties include flexibility, strength, biocompatibility, the capacity to maintain the function and viability of cells, and compatibility with bioprinting. Thus, improved compositions for tissue substitutes or bio-inks to generate the tissue substitutes are needed.

### SUMMARY

According to an aspect, a composition is provided herein. In some embodiments, the composition comprises: a hydrogel comprising collagen cross-linked with dialdehyde starch; and at least one population of cells comprising a plurality of chondrocytes, where the at least one population of cells is seeded on the hydrogel.

In some embodiments, the collagen is a Type I collagen. In some embodiments, the collagen is a Type II collagen, Type IX collagen, Type X collagen, or Type XI collagen. In some embodiments, the concentration of the collagen in the hydrogel is from about 0.1% to about 75% weight to volume. In some embodiments, the concentration of the collagen in the hydrogel is from about 0.5% to about 50% weight to volume. In some embodiments, the concentration of the collagen in the composition is from about 4% to about 8% weight to volume. In some embodiments, the collagen is derived from an animal. In some embodiments, the collagen is derived from skin.

In some embodiments, the concentration of the dialdehyde starch in the composition is from about 0.01% to about 15% weight to volume. In some embodiments, the concentration of the dialdehyde starch in the composition is from about 5% to about 10% weight to volume. In some embodiments, the concentration of the dialdehyde starch in the composition is about 10% weight to volume.

In some embodiments, the composition further comprises extracellular matrix (ECM). In some embodiments, the at least one population of cells comprises a plurality types of cells. In some embodiments, the at least one population of cells comprises at least three different types of cells, at least four different types of cells, or at least five different types of cells. In some embodiments, the at least one population of cells comprises two different types of cells, three different types of cells, four different types of cells, or five different types of cells. In some embodiments, the at least one population of cells is randomly distributed throughout at least 10%, at least 20%, at least 30%, at least 40%, at least 50%, at least 60%, at least 70%, at least 80%, at least 90%, or 100% of the volume of the hydrogel. In some embodiments, the at least one population of cells is evenly distributed on average throughout at least 10%, at least 20%, at least 30%, at least 40%, at least 50%, at least 60%, at least 70%, at least 80%, at least 90%, or 100% of the volume of the hydrogel. In some embodiments, the at least one population of cells comprises from about 1×10⁶ cells per mL of the hydrogel to about 50×10⁶ cells per mL of the hydrogel. In some embodiments, the at least one population of cells secretes an extracellular matrix protein.

In some embodiments, the at least one population of cells comprises at least one cell selected from the group consisting of: a synovial cell, a meniscus cell, an embryonic stem cell, a bone marrow-derived stem cell, an adipose-derived stromal cell, an infrapatellar fat pad-derived stem cell (IPFP), a pericyte, an induced pluripotent stem cell, a mesenchymal stem cell, an osteoblast, an endothelial cell, a human umbilical vein endothelial cell (HUVEC), and a myoblast.

In some embodiments, the at least one population of cells comprises at least one cell selected from the group consisting of: a human cell, a bovine cell, an equine cell, a murine cell, a canine cell, a feline cell, a lapine cell, and a porcine cell. In some embodiments, at least 80% of cells are viable in the composition.

In some embodiments, the hydrogel is a viscous gel. In some embodiments, the viscosity of the hydrogel is from 1 to 5000 centipoise. In some embodiments, the hydrogel has a mechanical stiffness ranging from 2 kPa to 1000 kPa. In some embodiments, the hydrogel has a mechanical stiffness ranging from 1000 kPa to 20 GPa.

In some embodiments, the hydrogel further comprises at least one growth factor. In some embodiments, the at least one growth factor comprises a plurality of different types of growth factors. In some embodiments, the plurality of different types of growth factors comprises at least three different types of growth factors, at least four different types of growth factors, or at least five different types of growth factors.

In some embodiments, the at least one growth factor comprises one type of growth factor, two different types of growth factors, three different types of growth factors, four different types of growth factors, or five different types of growth factors. In some embodiments, where the at least one growth factor is selected from the group consisting of PDGF-BB, PDGF-AA, PDGF-AB, PEDF, IGF, TGF-beta1, TGF-beta2, TGF-beta3, FGF, a BMP, EGF, GDF5, and a Wnt ligand.

In some embodiments, the composition further comprises heparin. In some embodiments, the heparin is conjugated to the collagen. In some embodiments, the heparin is conjugated to a growth factor of the at least one growth factor. In some embodiments, the hydrogel further comprises gelatin. In some embodiments, the ratio of gelatin to collagen in the hydrogel is 1:1 or less. In some embodiments, the ratio of gelatin to collagen in the hydrogel is 1:4 or less. In some embodiments, the composition further comprises a starch. In some embodiments, a concentration of starch in the composition is from 10% w/v to 20% w/v. In some embodiments, the starch is derived from corn.

In some embodiments, the hydrogel is injectable. In some embodiments, the hydrogel is moldable. In some embodiments, the composition is a bio-ink composition. In some embodiments, the hydrogel is moldable into a shape selected from the group consisting of: at least a portion of a meniscus, at least a portion of an articular cartilage, at least a portion of a rib, at least a portion of an ear, at least a portion of a nose, at least a portion of a bronchial tube, at least a portion of an intervertebral disc, at least a portion of a maxillofacial cartilage, at least a portion of a temporomandibular joint (TMJ), at least a portion of a glenoid labrum, and at least a portion of an acetabular labrum, a cylinder, a cube, a three-dimensional rectangle, a sphere, a crescent, and a tetrahedron. In some embodiments, the hydrogel is printed using a three-dimensional (3D) printer. In some embodiments, the hydrogel is printed into a shape selected from the group consisting of: at least a portion of a meniscus, at least a portion of an articular cartilage, at least a portion of a rib, at least a portion of an ear, at least a portion of a nose, at least a portion of a bronchial tube, at least a portion of an intervertebral disc, at least a portion of a maxillofacial cartilage, at least a portion of a temporomandibular joint (TMJ), at least a portion of a glenoid labrum, and at least a portion of an acetabular labrum, a cylinder, a cube, a three-dimensional rectangle, a sphere, a crescent, and a tetrahedron. In some embodiments, the hydrogel is lyophilized.

In another aspect, a layered composition is provided. The layered composition comprises a first layer comprising a first composition according to any of composition provided herein; and a second layer comprising a second composition according to any of composition provided herein, where the second composition is coupled to the first composition, where the first composition is different than the second composition by at least one of a mechanical property, a chemical property, and a biological property.

In some embodiments, the biological property is a type of cell in the composition, and where the at least one population of cells of the first layer comprises a different cell type than the at least one population of cells of the second layer.

In some embodiments, at least 40%, at least 50%, at least 60%, at least 70%, at least 80%, at least 90%, or at least 95% of collagen molecules of the first layer are oriented at an angle of 30 degrees or less relative to a first direction. The In some embodiments, at least 40%, at least 50%, at least 60%, at least 70%, at least 80%, at least 90%, or at least 95% of collagen molecules of the second layer are oriented at an angle 30 degrees or less relative to a second direction. In some embodiments, the first direction is oriented at an angle that is at least 10 degrees, at least 20 degrees, at least 30 degrees, at least 45 degrees, or at least 60 degrees relative to the second direction. In some embodiments, the first direction is perpendicular to the second direction. In some embodiments, the first layer is mechanically anisotropic.

In some embodiments, the second layer is mechanically anisotropic. In some embodiments, the second layer is mechanically is isotropic. In some embodiments, the concentration of the collagen in the first layer is from 0.5% to 50% weight to volume. In some embodiments, the concentration of the collagen in the first layer is from 4% to 8% weight to volume. In some embodiments, the concentration of the dialdehyde starch in the first layer is from 0.01% to 15% weight to volume. In some embodiments, the concentration of the dialdehyde starch in the first layer is from 5% to 10% weight to volume.

The layered composition of any one of claims 48-60, where the concentration of the dialdehyde starch in the first layer is 10% weight to volume.

In some embodiments, the concentration of the collagen in the second layer is from 0.5% to 50% weight to volume. In some embodiments, the concentration of the collagen in the second layer is from 4% to 8% weight to volume. In some embodiments, the concentration of the dialdehyde starch in the second layer is from 0.01% to 15% weight to volume. In some embodiments, the concentration of the dialdehyde starch in the second layer is from 5% to 10% weight to volume. In some embodiments, the concentration of the dialdehyde starch in the second layer is 10% weight to volume.

In some embodiments, the first layer comprises at least one growth factor. In some embodiments, the second layer comprises at least one growth factor. In some embodiments, the at least one growth factor comprises a plurality of different types of growth factors. In some embodiments, the plurality of different types of growth factors comprises at least three different types of growth factors, at least four different types of growth factors, or at least five different types of growth factors. In some embodiments, the at least one growth factor comprises two different types of growth factors, three different types of growth factors, four different types of growth factors, or five different types of growth factors. In some embodiments, the at least one growth factor is selected from the group consisting of PDGF-BB, PDGF-AA, PDGF-AB, PEDF, IGF, TGF-beta1, TGF-beta2, TGF-beta3, FGF, a BMP, EGF, GDF5, and a Wnt ligand. In some embodiments, the ratio of gelatin to collagen in the hydrogel is 1:4 or less. In some embodiments, a growth factor of the at least one growth factor is conjugated with heparin. In some embodiments, the composition further comprises a buffer. In some embodiments, the buffer is a zwitterionic buffer.

In some embodiments, the buffer is selected from the group consisting of: MES, ADA, PIPES, ACES, MOPSO, MOPS, BES, TES, HEPES, DIPSO, Acetamidoglycine, TAPSO, POPSO, HEPPSO, HEPPS, Tricine, Glycinamide, Bicine, and TAPS.

In another aspect, a method of forming a hydrogel is provided. The method comprises mixing collagen with dialdehyde starch and a population of cells comprising a plurality of chondrocytes.

In some embodiments, the method further comprises mixing a starch. In some embodiments, a concentration of starch in the hydrogel is from 10% w/v to 20% w/v. In some embodiments, the starch is derived from corn. In some embodiments, the collagen is Type I collagen. In some embodiments, the collagen is Type II collagen, Type IX collagen, Type X collagen, or Type XI collagen. In some embodiments, the concentration of the collagen in the hydrogel is from 0.1% to 75% weight to volume. In some embodiments, the concentration of the collagen in the hydrogel is from 0.5% to 50% weight to volume. In some embodiments, the concentration of the collagen in the composition is from 4% to 8% weight to volume. In some embodiments, the collagen is derived from an animal. In some embodiments, the collagen is derived from skin. In some embodiments, the concentration of the dialdehyde starch in the composition is from 0.01% to 15% weight to volume. In some embodiments, the concentration of the dialdehyde starch in the composition is from 5% to 10% weight to volume. In some embodiments, the concentration of the dialdehyde starch in the composition is about 10% weight to volume.

In some embodiments, the method further comprises adding extracellular matrix (ECM) to the mixing. In some embodiments, the at least one population of cells comprises a plurality types of cells. In some embodiments, the at least one population of cells comprises at least three different types of cells, at least four different types of cells, or at least five different types of cells. In some embodiments, the at least one population of cells comprises two different types of cells, three different types of cells, four different types of cells, or five different types of cells. In some embodiments, the at least one population of cells is randomly distributed throughout at least 10%, at least 20%, at least 30%, at least 40%, at least 50%, at least 60%, at least 70%, at least 80%, at least 90%, or 100% of the volume of the hydrogel. In some embodiments, the at least one population of cells is evenly distributed on average throughout at least 10%, at least 20%, at least 30%, at least 40%, at least 50%, at least 60%, at least 70%, at least 80%, at least 90%, or 100% of the volume of the hydrogel. In some embodiments, the at least one population of cells comprises from about 1×10⁶ cells per mL of the hydrogel to about 50×10⁶ cells per mL of the hydrogel. In some embodiments, the at least one population of cells comprises at least one cell selected from the group consisting of: a synovial cell, a meniscus cell, an embryonic stem cell, a bone marrow-derived stem cell, an adipose-derived stromal cell, an infrapatellar fat pad-derived stem cell (IPFP), a pericyte, an induced pluripotent stem cell, a mesenchymal stem cell, an osteoblast, an endothelial cell, a human umbilical vein endothelial cell (HUVEC), and a myoblast. In some embodiments, the at least one population of cells comprises at least one cell selected from the group consisting of: a human cell, a bovine cell, an equine cell, a murine cell, a canine cell, a feline cell, a lapine cell, and a porcine cell. In some embodiments, the at least one population of cells secretes an extracellular matrix protein.

In another aspect, a method of mixing is provided. In some embodiments, the method of the mixing comprises steps of: generating a first mixture of collagen and at least one population of cells comprising a plurality of chondrocytes; generating the hydrogel by adding dialdehyde starch to the first mixture.

In some embodiments, the method of mixing further comprises neutralizing a pH of the hydrogel while the collagen, the population of cells, and the dialdehyde starch are mixed. In some embodiments, the neutralizing comprises adding a buffer. In some embodiments, the buffer is zwitterionic. In some embodiments, the buffer is selected from the group consisting of: MES, ADA, PIPES, ACES, MOPSO, MOPS, BES, TES, HEPES, DIPSO, Acetamidoglycine, TAPSO, POPSO, HEPPSO, HEPPS, Tricine, Glycinamide, Bicine, and TAPS. In some embodiments, the hydrogel is a viscous gel. In some embodiments, the viscosity of the hydrogel is from 1 to 5000 centipoise. In some embodiments, the hydrogel has a mechanical stiffness ranging from 2 kPa to 1000 kPa. In some embodiments, the hydrogel has a mechanical stiffness ranging from 1000 kPa to 20 GPa. In some embodiments, the hydrogel further comprises at least one growth factor. In some embodiments, the at least one growth factor comprises a plurality of different types of growth factors. In some embodiments, the plurality of different types of growth factors comprises at least three different types of growth factors, at least four different types of growth factors, or at least five different types of growth factors.

In some embodiments, n the at least one growth factor comprises two different types of growth factors, three different types of growth factors, four different types of growth factors, or five different types of growth factors. In some embodiments, the at least one growth factor is selected from the group consisting of PDGF-BB, PDGF-AA, PDGF-AB, PEDF, IGF, TGF-beta1, TGF-beta2, TGF-beta3, FGF, a BMP, EGF, GDF5, and a Wnt ligand.

In some embodiments, the method further comprises adding heparin. In some embodiments, the heparin is conjugated to the collagen. In some embodiments, where the heparin is conjugated to a growth factor of the at least one growth factor. In some embodiments, where the hydrogel further comprises gelatin. In some embodiments, the ratio of gelatin to collagen in the hydrogel is 1:1 or less. In some embodiments, the ratio of gelatin to collagen in the hydrogel is 1:4 or less.

In some embodiments, the collagen, the dialdehyde starch, and the population of cells are mixed in a mold. In some embodiments, a shape of the mold comprises at least a portion of a meniscus, at least a portion of an articular cartilage, at least a portion of a rib, at least a portion of an ear, at least a portion of a nose, at least a portion of a bronchial tube, at least a portion of an intervertebral disc, at least a portion of a maxillofacial cartilage, at least a portion of a temporomandibular joint (TMJ), at least a portion of a glenoid labrum, at least a portion of an acetabular labrum, a cylinder, or any combinations thereof.

In some embodiments, the method further comprises extruding a pattern onto a substrate with the collagen, the dialdehyde starch, and the population of cells as they are mixed.

In some embodiments, the method further comprises depositing the collagen, the dialdehyde starch, and the population of cells as they are mixed in a predetermined shape onto a substrate. In some embodiments, where at least 80% of the populations of cells are viable. In some embodiments, the substrate comprises a target region of a subject. In some embodiments, the target region comprises at least a portion of a knee joint, at least a portion of a shoulder joint, at least a portion of a hip joint, at least a portion of a temporomandibular joint (TMJ), at least a portion of a rib, at least a portion of an ear, at least a portion of a nose, at least a portion of a bronchial tube, at least a portion of an intervertebral disc, or at least a portion of a maxillofacial cartilage. In some embodiments, the at least a portion of a knee joint comprises a meniscus. In some embodiments, the at least a portion of a shoulder joint comprises a glenoid labrum. In some embodiments, the at least a portion of a hip joint comprises an acetabular labrum. In some embodiments, the at least a portion of a temporomandibular joint comprises a maxillofacial cartilage. In some embodiments, the predetermined shape is at least 50%, at least 60%, at least 70%, at least 80%, or at least 90% identical to the target region.

In another aspect, a method of forming a hydrogel scaffold is provided. In some embodiments, the method comprises: mixing collagen with dialdehyde starch to cross-link the collagen into a hydrogel; and seeding the cross-linked collagen with at least one population of cells comprising a plurality of chondrocytes.

In some embodiments, the method further comprises mixing a starch. In some embodiments, a concentration of starch in the hydrogel is from 10% w/v to 20% w/v. In some embodiments, the starch derived from corn.

In some embodiments, the collagen is Type I collagen. In some embodiments, the collagen is Type II collagen, Type IX collagen, Type X collagen, or Type XI collagen. In some embodiments, the concentration of the collagen in the hydrogel is from 0.1% to 75% weight to volume. In some embodiments, the concentration of the collagen in the hydrogel is from 0.5% to 50% weight to volume. In some embodiments, the concentration of the collagen in the composition is from 4% to 8% weight to volume. In some embodiments, the collagen is derived from an animal. In some embodiments, the collagen is derived from skin.

In some embodiments, the concentration of the dialdehyde starch in the composition is from 0.01% to 15% weight to volume. In some embodiments, the concentration of the dialdehyde starch in the composition is from 5% to 10% weight to volume. In some embodiments, the concentration of the dialdehyde starch in the composition is about 10% weight to volume.

In some embodiments, the method further comprises mixing extracellular matrix (ECM). In some embodiments, the at least one population of cells comprises a plurality types of cells. In some embodiments, the at least one population of cells comprises at least three different types of cells, at least four different types of cells, or at least five different types of cells. In some embodiments, the at least one population of cells comprises two different types of cells, three different types of cells, four different types of cells, or five different types of cells. In some embodiments, the at least one population of cells is randomly seeded throughout at least 10%, at least 20%, at least 30%, at least 40%, at least 50%, at least 60%, at least 70%, at least 80%, at least 90%, or 100% of the volume of the hydrogel. In some embodiments, the at least one population of cells is evenly seeded on average throughout at least 10%, at least 20%, at least 30%, at least 40%, at least 50%, at least 60%, at least 70%, at least 80%, at least 90%, or 100% of the volume of the hydrogel. In some embodiments, the at least one population of cells is seeded in a pattern within at least 10%, at least 20%, at least 30%, at least 40%, at least 50%, at least 60%, at least 70%, at least 80%, at least 90%, or 100% of the volume of the hydrogel. In some embodiments, the at least one population of cells comprises from about 1×10⁶ cells per mL of the hydrogel to about 50×10⁶ cells per mL of the hydrogel. In some embodiments, the at least one population of cells comprises at least one cell selected from the group consisting of: a synovial cell, a meniscus cell, an embryonic stem cell, a bone marrow-derived stem cell, an adipose-derived stromal cell, an infrapatellar fat pad-derived stem cell (IPFP), a pericyte, an induced pluripotent stem cell, a mesenchymal stem cell, an osteoblast, an endothelial cell, a human umbilical vein endothelial cell (HUVEC), and a myoblast. In some embodiments, the at least one population of cells comprises at least one cell selected from the group consisting of: a human cell, a bovine cell, an equine cell, a murine cell, a canine cell, a feline cell, a lapine cell, and a porcine cell. In some embodiments, the at least one population of cells secretes an extracellular matrix protein.

In some embodiments, the method further comprises neutralizing a pH of the hydrogel before seeding the cross-linked collagen with the at least one population of cells. In some embodiments, the neutralizing comprises adding a buffer. In some embodiments, the buffer is zwitterionic. In some embodiments, the buffer is selected from the group consisting of: MES, ADA, PIPES, ACES, MOPSO, MOPS, BES, TES, HEPES, DIPSO, Acetamidoglycine, TAPSO, POPSO, HEPPSO, HEPPS, Tricine, Glycinamide, Bicine, and TAPS.

In some embodiments, the method further comprises placing the hydrogel into a mold. In some embodiments, the method further comprises placing the hydrogel scaffold into a mold.

In some embodiments, a shape of the mold comprises at least a portion of a meniscus, at least a portion of an articular cartilage, at least a portion of a rib, at least a portion of an ear, at least a portion of a nose, at least a portion of a bronchial tube, at least a portion of an intervertebral disc, at least a portion of a maxillofacial cartilage, at least a portion of a temporomandibular joint (TMJ), at least a portion of a glenoid labrum, at least a portion of an acetabular labrum, a cylinder, or any combinations thereof.

In some embodiments, the collagen is conjugated with a heparin.

In some embodiments, the heparin is conjugated with at least one growth factor. In some embodiments, at least two different growth factors, at least three different growth factors, at least four different growth factors, or at least five different growth factors are conjugated to the heparin. In some embodiments, two different growth factors, three different growth factors, four different growth factors, or five different growth factors are conjugated to the heparin. In some embodiments, the growth factor is selected from the group consisting of PDGF-BB, PDGF-AA, PDGF-AB, PEDF, IGF, TGF-beta1, TGF-beta2, TGF-beta3, FGF, a BMP, EGF, GDF5, and a Wnt ligand. In some embodiments, the hydrogel further comprises gelatin. In some embodiments, the ratio of gelatin to collagen in the hydrogel is 1:1 or less.

In some embodiments, the hydrogel is a viscous gel. In some embodiments, the viscosity of the hydrogel is from 1 to 5000 centipoise. In some embodiments, the hydrogel is injectable. In some embodiments, the hydrogel has a mechanical stiffness ranging from 2 kPa to 1000 kPa. In some embodiments, the hydrogel has a mechanical stiffness ranging from 1000 kPa to 20 GPa.

In another aspect, the method comprises forming a layered hydrogel by forming a first layer comprising a hydrogel comprising a composition according to any composition provided herein and a second layer comprising a composition according to any composition provided herein, where the composition of the first layer is different than the composition of the second layer by at least one of a mechanical property, a chemical property, and a biological property.

In some embodiments, the biological property is a type of cell in the composition, and where the at least one population of cells of the first layer comprises a different cell type than the at least one population of cells of the second layer. In some embodiments, at least 40%, at least 50%, at least 60%, at least 70%, at least 80%, at least 90%, or at least 95% of collagen molecules of the first layer are oriented at an angle of 30 degrees or less relative to a first direction. In some embodiments, at least 40%, at least 50%, at least 60%, at least 70%, at least 80%, at least 90%, or at least 95% of collagen molecules of the second layer are oriented at an angle 30 degrees or less relative to a second direction.

In some embodiments, the first direction is oriented at an angle that is at least 10 degrees, at least 20 degrees, at least 30 degrees, at least 45 degrees, or at least 60 degrees relative to the second direction. In some embodiments, the first direction is perpendicular to the second direction. In some embodiments, the second layer is mechanically anisotropic. In some embodiments, the second layer is mechanically is isotropic.

In some embodiments, the concentration of the collagen in the first layer is from 0.5% to 50% weight to volume. In some embodiments, the concentration of the collagen in the first layer is from 4% to 8% weight to volume. In some embodiments, the concentration of the dialdehyde starch in the first layer is from 0.01% to 15% weight to volume. In some embodiments, the concentration of the dialdehyde starch in the first layer is from 5% to 10% weight to volume. In some embodiments, the concentration of the dialdehyde starch in the first layer is 10% weight to volume. In some embodiments, the concentration of the collagen in the second layer is from 0.5% to 50% weight to volume. In some embodiments, the concentration of the collagen in the second layer is from 4% to 8% weight to volume. In some embodiments, the concentration of the dialdehyde starch in the second layer is from 0.01% to 15% weight to volume. In some embodiments, the concentration of the dialdehyde starch in the second layer is from 5% to 10% weight to volume. In some embodiments, the concentration of the dialdehyde starch in the second layer is 10% weight to volume.

In some embodiments, the first layer comprises at least one growth factor. In some embodiments, the second layer comprises at least one growth factor. In some embodiments, the at least one growth factor comprises a plurality of different types of growth factors. In some embodiments, the plurality of different types of growth factors comprises at least three different types of growth factors, at least four different types of growth factors, or at least five different types of growth factors. In some embodiments, the at least one growth factor comprises two different types of growth factors, three different types of growth factors, four different types of growth factors, or five different types of growth factors. In some embodiments, the at least one growth factor is selected from the group consisting of PDGF-BB, PDGF-AA, PDGF-AB, PEDF, IGF, TGF-beta1, TGF-beta2, TGF-beta3, FGF, a BMP, EGF, GDF5, and a Wnt ligand. In some embodiments, a growth factor of the at least one growth factor is conjugated with heparin.

In some embodiments, the method further comprises adding a buffer. In some embodiments, the buffer is a zwitterionic buffer. In some embodiments, the buffer is selected from the group consisting of: MES, ADA, PIPES, ACES, MOPSO, MOPS, BES, TES, HEPES, DIPSO, Acetamidoglycine, TAPSO, POPSO, HEPPSO, HEPPS, Tricine, Glycinamide, Bicine, and TAPS.

In some embodiments, the first and second layers are in different shapes. In some embodiments, the first and second layers are in different pH. In some embodiments, the first and second layers have different stiffness.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the subject matter disclosed herein are set forth with particularity in the appended claims. A better understanding of the features and advantages of the subject matter disclosed herein will be obtained by reference to the following detailed description that sets forth illustrative embodiments, in which the principles of the subject matter disclosed herein are utilized, and the accompanying drawings of which:

FIG. 1A shows unseeded and cell-seeded dialdehyde starch-collagen (DAS-COL) disc-shaped hydrogels at Day 0, in accordance with embodiments.

FIG. 1B shows unseeded and cell-seeded dialdehyde starch-collagen (DAS-COL) disc-shaped hydrogels at 2 weeks in culture, in accordance with embodiments.

FIG. 2A shows disc-shaped dialdehyde starch-collagen hydrogels seeded with bovine chondrocytes at Day 0, in accordance with embodiments.

FIG. 2B shows the disc-shaped, chondrocyte-seeded dialdehyde starch-collagen hydrogels of FIG. 2A at Day 1 of culture in insulin-transferrin-sodium selenite (ITS) supplemented medium (left culture well) or in medium supplemented with ITS and TGF-□3 (right culture well), in accordance with embodiments.

FIG. 2C shows the disc-shaped, chondrocyte-seeded dialdehyde starch-collagen hydrogels of FIG. 2A at Day 7 of culture in insulin-transferrin-sodium selenite (ITS) supplemented medium (left culture well) or in medium supplemented with ITS and TGF-□3 (right culture well), in accordance with embodiments.

FIG. 2D shows the disc-shaped, chondrocyte-seeded dialdehyde starch-collagen hydrogels of FIG. 2A at Day 14 of culture in insulin-transferrin-sodium selenite (ITS) supplemented medium (left culture well) or in medium supplemented with ITS and TGF-□3 (right culture well), in accordance with embodiments.

FIG. 2E shows a disc-shaped chondrocyte-seeded dialdehyde starch-collagen hydrogel after 14 days of culture in insulin-transferrin-sodium selenite (ITS) supplemented medium (left image) or in medium supplemented with ITS and TGF-□3 (right image), in accordance with embodiments.

FIG. 3A shows a method for seeding a hydrogel with cells, in accordance with embodiments.

FIG. 3B shows evaluation of cell viability in a hydrogel, in accordance with embodiments.

FIGS. 3C-3E show exemplary devices for mixing components of hydrogels and assembly thereof, in accordance with embodiments.

FIG. 3F shows a method for mixing components of a hydrogel, in accordance with embodiments.

FIGS. 4A-4E show cell viability staining for dialdehyde starch-collagen (DAS-COL) hydrogels comprising bovine chondrocytes after one or two days of culture with initial seeding densities of 1 million cells/mL (FIG. 4A), 2 million cells/mL (FIG. 4B), 4 million cells/mL (FIG. 4C), 8 million cells/mL (FIG. 4D), or 10 million cells/mL (FIG. 4E), in accordance with embodiments (arrows indicate cells stained as non-viable; scale bars indicate 50 □m).

FIG. 5A shows a fluorescent microscopy image of a human meniscal cell-seeded hydrogel comprising 0% DAS-100% COL stained with a cell viability dye after 3 days in culture (10× magnification), in accordance with embodiments.

FIG. 5B shows a fluorescent microscopy image of a human meniscal cell-seeded hydrogel comprising 10% DAS-90% COL stained with a cell viability dye after 3 days in culture (10× magnification), in accordance with embodiments.

FIG. 5C shows a fluorescent microscopy image of a human meniscal cell-seeded hydrogel comprising 25% DAS-75% COL stained with a cell viability dye after 3 days in culture (10× magnification), in accordance with embodiments.

FIG. 5D shows a fluorescent microscopy image of a human meniscal cell-seeded hydrogel comprising 50% DAS-50% COL stained with a cell viability dye after 3 days in culture (10× magnification), in accordance with embodiments.

FIG. 5E shows a fluorescent microscopy image of a human meniscal cell-seeded hydrogel comprising 75% DAS-25% COL stained with a cell viability dye after 3 days in culture (10× magnification), in accordance with embodiments.

FIGS. 6A-6D show fluorescent microscopy images of human chondrocytes embedded within DAS-COL hydrogels and stained with a cell viability dye, in accordance with embodiments. FIG. 6A shows a microscopy image of the DAS-COL hydrogel comprising human chondrocytes at Day 1. FIG. 6B shows a microscopy image of the DAS-COL hydrogel comprising human chondrocytes at Day 9. FIG. 6C shows a microscopy image of the DAS-COL hydrogel comprising human chondrocytes at Day 21 at a high magnification. FIG. 6D shows a microscopy image of the DAS-COL hydrogel comprising human chondrocytes at Day 21 at a lower magnification than FIG. 6C. Positive staining in FIGS. 6A-6D indicates positive viability except for cells indicated with arrowheads, which stained positive for ethidium homodimer-1.

FIG. 7 shows Fourier transform infrared (FTIR) spectroscopy data obtained from analysis of collagen (COL) matrix, dialdehyde starch-collagen hydrogel (DAS-COL), and dialdehyde starch-collagen-heparin (DAS-COL-HEP) hydrogel samples, in accordance with embodiments.

FIGS. 8A-8D show photographs of the DAS-COL hydrogel formation and different physical properties of DAS-COL hydrogels. FIG. 8A shows a photograph of the DAS-COL hydrogel after 10 minutes of being mixed. FIG. 8B shows a photograph of the DAS-COL hydrogel after 1 hour of being mixed. FIG. 8C shows a photograph of the DAS-COL hydrogel after 3 hours of being mixed. FIG. 8D shows the change in Young's moduli based on increasing cross-linking time.

FIGS. 9A-9C show hydrogels formed in the presence of buffers of different concentrations of NaOH, in accordance with embodiments. FIG. 9A shows a DAS-COL hydrogel formed in the presence of 0.25 N NaOH. FIG. 9B shows a DAS-COL hydrogel formed in the presence of 0.5 N NaOH. FIG. 9C shows a DAS-COL hydrogel formed in the presence of 0.75 N NaOH.

FIG. 9D shows measured Young's modulus values for DAS-COL hydrogels formed for 3 hours in the presence of 0.25 N NaOH, 0.5 N NaOH, and 0.75 N NaOH, in accordance with embodiments.

FIG. 10 shows a mechanical characterization of the DAS-COL hydrogels: change in Young's moduli based as a result of different collagen to gelatin ratios.

FIG. 11 shows a suture test of a DAS-COL-HEP (8% COL w/v) hydrogel.

FIGS. 12A-12E show degradation of DAS-COL hydrogels (10% w/v DAS, 4% w/v COL; 10% w/v DAS, 6% w/v COL; 10% w/v DAS, 8% w/v COL) in 0 days (FIG. 12A), 1 day (FIG. 12B), 7 days (FIG. 12C), 14 days (FIG. 12D), or 21 days (FIG. 12E) in the presence of 0.75 N NaOH at 37° C.

FIG. 13A shows a 10× image of a Safranin O stained histological section of a chondrocyte-seeded DAS-COL hydrogel cultured in medium supplemented with insulin-transferrin-sodium selenite (ITS), in accordance with embodiments.

FIG. 13B shows a 40× image of the histological section of FIG. 13A.

FIG. 13C shows a 10× image of a Safranin O stained histological section of a chondrocyte-seeded DAS-COL hydrogel cultured in medium supplemented with insulin-transferrin-sodium selenite (ITS) and TGF-□3, in accordance with embodiments.

FIG. 13D shows a 40× image of a histological section of a chondrocyte-seeded DAS-COL hydrogel cultured in medium supplemented with insulin-transferrin-sodium selenite (ITS) and TGF-□3, in accordance with embodiments.

FIGS. 14A-14F show histological images of hydrogels, in accordance with embodiments. FIG. 14A shows Safranin-O staining of a section of a DAS-COL-HEP hydrogel seeded with bovine chondrocytes and cultured for 21 days. FIG. 14B shows a magnified portion of the DAS-COL-HEP hydrogel shown in FIG. 14A. 14A stained with a viability dye. FIG. 14C shows Safranin-O staining of a section of a DAS-COL-HEP-IGF1 hydrogel seeded with bovine chondrocytes and cultured for 21 days. FIG. 14D shows a magnified portion of the DAS-COL-HEP-IGF1 hydrogel shown in FIG. 14C. FIG. FIG. 14E shows Safranin-O staining of a section of a DAS-COL-HEP-TGF□3 hydrogel that has been cultured for 21 days. FIG. 14F shows a magnified portion of the DAS-COL-HEP-TGF□3 hydrogel shown in FIG. 14E.

FIGS. 15A-15I show fluorescent images of hydrogels, in accordance with embodiments. FIG. 15A shows fluorescent viability staining of a section of a DAS-COL hydrogel seeded with bovine chondrocytes at a concentration of 10 million cell/mL that has been cultured for 1 day. FIG. 15B shows fluorescent viability staining of a section of a DAS-COL hydrogel seeded with bovine chondrocytes at a concentration of 10 million cell/mL that has been cultured for 9 days. FIG. 15C shows fluorescent viability staining of a section of a DAS-COL hydrogel seeded with bovine chondrocytes at a concentration of 10 million cell/mL that has been cultured for 21 days. FIG. 15D shows fluorescent viability staining of a section of a DAS-COL-IGF1 hydrogel seeded with bovine chondrocytes at a concentration of 10 million cell/mL that has been cultured for 1 day. FIG. 15E shows fluorescent viability staining of a section of a DAS-COL-IGF1 hydrogel seeded with bovine chondrocytes at a concentration of 10 million cell/mL that has been cultured for 9 days. FIG. 15F shows fluorescent viability staining of a section of a DAS-COL-IGF1 hydrogel seeded with bovine chondrocytes at a concentration of 10 million cell/mL that has been cultured for 21 days. FIG. 15G shows fluorescent viability staining of a section of a DAS-COL-TGF D 1 hydrogel seeded with bovine chondrocytes at a concentration of 10 million cell/mL that has been cultured for 1 day. FIG. 15H shows fluorescent viability staining of a section of a DAS-COL-TGF D 1 hydrogel seeded with bovine chondrocytes at a concentration of 10 million cell/mL that has been cultured for 9 days. FIG. 15I shows fluorescent viability staining of a section of a DAS-COL-TGF D 1 hydrogel seeded with bovine chondrocytes at a concentration of 10 million cell/mL that has been cultured for 21 days.

FIGS. 16A-16B show fluorescent images of DAS-COL-IGF1 hydrogels seeded with bovine chondrocytes and cultured for 21 days, in accordance with embodiments. FIG. 16B shows a magnified portion of the hydrogel section shown in FIG. 16A. FIGS. 16A and 16B show fluorescence-labeled 40 kDa dextran conjugated to IGF-1 of the hydrogel; punctate staining in FIGS. 16A and 16B indicates cellular nuclei (Hoechst staining).

FIGS. 17A-17G show a schematic diagram of cell migration assay and images from a migration assay for a DAS-COL-HEP hydrogel and a DAS-COL-HEP-PDGFBB hydrogel, in accordance with embodiments.

FIG. 18A shows toluidine blue staining of DAS-COL-HEP hydrogels printed onto a hydrophobic poly-lactic acid (PLA) sheet. Portions of the PLA sheet were printed with DAS-COL hydrogels that has been mixed with heparin sodium salt (0.1% w/v) either before (left) or after (right) a pH neutralization step was performed on the DAS-COL.

FIG. 18B shows toluidine blue staining of a DAS-COL-HEP hydrogel where heparin was mixed with DAS-COL before the DAS-COL mixture was subjected to a pH neutralization step.

FIG. 18C shows toluidine blue staining of a DAS-COL-HEP hydrogel where heparin was mixed with DAS-COL after the DAS-COL mixture was subjected to a pH neutralization step.

FIG. 18D shows quantification of toluidine blue staining shown in FIGS. 18B and 18C.

FIGS. 19A-19D show devices for formation of DAS-COL hydrogels and sponges and formed DAS-COL hydrogels and sponges, in accordance with embodiments. FIG. 19A shows addition of a growth factor to DAS-COL prior to a neutralization step, in accordance with embodiments. FIG. 19B shows molding of DAS-COL-GF hydrogels into cylindrical plugs, in accordance with embodiments. FIG. 19C shows a blocking step applied to DAS-COL-GF hydrogels, in accordance with embodiments. FIG. 19D shows lyophilized DAS-COL-GF sponges, in accordance with embodiments.

FIG. 20A shows a DAS-COL-CHON hydrogel comprising two layers after 21 days in culture, in accordance with embodiments. The left portion (e.g., left layer, arrow) of the hydrogel comprises IGF-1 conjugated to the DAS-COL-CHON hydrogel matrix (DAS-COL-CHON-IGF1), and the right portion (e.g. right layer, double arrows) of the hydrogel comprises TGF-□3 conjugated to the DAS-COL-CHON hydrogel matrix, in accordance with embodiments.

FIG. 20B shows fluorescent staining of the DAS-COL-CHON-IGF1 hydrogel layer shown in FIG. 20A, in accordance with embodiments. Fluorescent staining indicates 40 kDa dextran conjugated to IGF-1 of the hydrogel; punctate staining indicates cellular nuclei (Hoechst staining).

FIG. 20C shows fluorescent staining of the DAS-COL-CHON-TGF D 3 hydrogel layer shown in FIG. 20A, in accordance with embodiments. Fluorescent staining indicates 40 kDa dextran conjugated to TGF-□3 of the hydrogel; punctate staining indicates cellular nuclei (Hoechst staining)

FIG. 21A shows three-layer DAS-COL hydrogels with chondrocytes, in accordance with some embodiments.

FIG. 21B shows a fluorescent image of a cross-section of a three layer DAS-COL hydrogel with chondrocytes.

FIGS. 22A-22D show photographs of the DAS-COL hydrogels, in accordance with embodiments. FIG. 22A shows an image of the DAS-COL hydrogel loaded in a syringe. FIG. 22B shows twenty-four DAS-COL hydrogels with cells embedded that were extruded into a disc shape via printing and further cultured in vitro. FIG. 22C shows an image of a DAS-COL hydrogel that was extruded in the shape of a cylindrical disc. FIG. 22D shows an image of a DAS-COL hydrogel that was molded in the shape of a nose.

FIGS. 23A-23G show DAS-COL hydrogels, in accordance with embodiments. FIG. 23A shows printed DAS-COL hydrogels using a 1% DAS weight to volume (w/v) and a 4% COL w/v mixed at a ratio of 1 DAS:9 COL, FIG. 23B shows a 1% DAS, 4% COL w/v mixed at a ratio of 3 DAS:7 COL, and FIG. 23C shows a 1% DAS, 4% COL w/v mixed at a ratio of 5 DAS:5 COL. FIG. 23D shows printed DAS-COL hydrogels using a 1% DAS and an 8% COL w/v mixed at ratios of 2 DAS: 8 COL and FIG. 23E shows a 1% DAS and an 8% COL w/v mixed at ratios of 1 DAS: 9 COL. FIG. 23F shows molded DAS-COL hydrogel using a 10% DAS w/v and a 4% COL w/v mixed at a ratio of 1 DAS:9 COL. FIG. 23G shows a molded DAS-COL hydrogel using a 10% DAS and an 8% COL w/v mixed at a ratio of 1 DAS:9 COL, in accordance with embodiments.

FIG. 24A shows DAS-COL-CHON hydrogels comprising 10 million bovine chondrocytes per mL hydrogel, in accordance with embodiments. The matrix of the top (control) DAS-COL hydrogel is not conjugated to growth factors prior to implantation. The matrix of the bottom DAS-COL hydrogel is conjugated to IGF-1 and TGF-□3 prior to implantation.

FIG. 24B shows a histologically stained section of a DAS-COL-CHON hydrogel that is not conjugated to growth factors prior to implantation into a bovine knee defect (as shown in the top hydrogel of FIG. 24A) from a sample taken three weeks after implantation. Dotted lines indicate edges of bovine knee defect.

FIG. 24C shows a high magnification view of the stained section shown in FIG. 24B.

FIG. 24D shows a histologically stained section of a DAS-COL-CHON hydrogel that is conjugated to IGF-1 and TGF-□3 prior to implantation into a bovine knee defect (as shown in the bottom hydrogel of FIG. 24A) from a sample taken three weeks after implantation. Dotted lines indicate edges of the bovine knee defect.

FIG. 24E shows a high magnification view of the stained section shown in FIG. 24D.

FIG. 25A shows an example of a collagen solution, in accordance with some embodiments.

FIG. 25B shows an example of a collagen solution with added starch, in accordance with some embodiments.

FIG. 25C shows an example of DAS-COL mixture in a syringe, in accordance with some embodiments.

FIGS. 25D-25E shows an example of a method to mix DAS-COL and starch, in accordance with some embodiments

FIG. 25F shows an example of a DAS-COL and starch crosslinked composition in a neutralized state, in accordance with some embodiments.

FIG. 26A show an example of a crosslinked collagen composition, in accordance with some embodiments.

FIG. 26B show an example of a neutralized collagen composition, in accordance with some embodiments.

FIG. 27A show an example of a 3D printed shape using bioink material comprising DAS-COL crosslinked with starch, in accordance with some embodiments.

FIG. 27B show examples of another 3D printed shape using bioink material comprising DAS-COL crosslinked with starch, in accordance with some embodiments.

FIG. 27C show examples of disc-shaped hydrogels made from DAS-COL crosslinked with starch, in accordance with some embodiments.

FIG. 27D shows examples of lyophilized disc-shaped DAS-COL hydrogels, in accordance with some embodiments.

FIG. 28A shows examples of printed DAS-COL hydrogel mixed with 10% starch, in accordance with some embodiments.

FIG. 28B shows examples of printed DAS-COL hydrogel mixed with 20% starch, in accordance with some embodiments.

FIGS. 28C-28D show examples of printed cultured DAS-COL hydrogel mixed with starch in the PBS at Day 1, in accordance with some embodiments.

FIGS. 28E-28F show examples of printed cultured DAS-COL hydrogel mixed with starch in the PBS at Day 7, in accordance with some embodiments.

FIGS. 29A-29E show an example of a DAS-COL degradation test over time, in accordance with some embodiments.

FIG. 30A shows an example of cultured DAS-COL extruded gel in the media, in accordance with some embodiments.

FIG. 30B shows an example of live cell fluorescent imaging in a cultured DAS-COL extruded gel, in accordance with some embodiments.

FIG. 30C shows an example of dead cell fluorescent imaging in a cultured DAS-COL extruded gel, in accordance with some embodiments.

FIG. 31A shows an example of cultured DAS-COL-starch extruded gel in the media, in accordance with some embodiments.

FIG. 31B shows an example of live cell fluorescent imaging in a cultured DAS-COL-starch extruded gel, in accordance with some embodiments.

FIG. 31C shows an example of dead cell fluorescent imaging in a cultured DAS-COL-starch extruded gel, in accordance with some embodiments.

FIG. 32A shows printed DAS-COL-STARCH gel with 27G needle, in accordance with some embodiments.

FIG. 32B shows printed DAS-COL-STARCH gel with 20G needle, in accordance with some embodiments.

FIG. 33A shows a defect made in agarose gel.

FIG. 33B shows laser scanning of the surface of the defect.

FIGS. 33C-33D show examples of bio-printed DAS-COL-based gel in agarose from top-view and a bottom view, in accordance with some embodiments.

FIG. 33E show an example of bio-printed DAS-COL-based gel in agarose from side-view, in accordance with some embodiments.

FIGS. 33F-33G show examples of compression testing of a bio-printed gel at two different time points, in accordance with some embodiments.

FIG. 34A shows an example of a lyophilized DAS-COL sponge, in accordance with some embodiments.

FIG. 34B shows an example of cell culture in DAS-COL sponge, in accordance with some embodiments.

FIG. 34C shows side view of an example of a cultured DAS-COL sponge, in accordance with some embodiments.

FIG. 34D shows an example of a section view of a cultured DAS-COL sponge, in accordance with some embodiments.

FIG. 34E shows an example of a DAS-COL sponge loaded with cells and cell culture media, in accordance with some embodiments.

FIG. 34F show an example of live cell imaging in a DAS-COL sponge loaded with cells and cell culture media, in accordance with some embodiments.

FIG. 34G show an example of dead cell imaging in a DAS-COL sponge loaded with cells and cell culture media, in accordance with some embodiments.

FIGS. 35A-35B show a compression test of an example of a DAS-COL gel, in accordance with some embodiments.

FIG. 36 shows an example of a mechanical characterization of the DAS-COL hydrogels as a result of different collagen to gelatin ratios.

FIG. 37A shows an example of parameters used in a bioprinting process, in accordance with some embodiments.

FIG. 37B shows an example of a bioprinting process, in accordance with some embodiments.

FIG. 38A shows an example of parameters used in a bioprinting process, in accordance with some embodiments.

FIG. 38B show another example of a bioprinting process, in accordance with some embodiments.

FIG. 39A shows an example of extruded DAS-COL-STARCH encapsulated with HUVEC on 24-well plate, in accordance with some embodiments.

FIG. 39B shows an example of cell viability in DAS-COL-STARCH encapsulating HUVEC, in accordance with some embodiments.

FIG. 39C shows an example of cell imaging of dead cells in DAS-COL-STARCH encapsulated with HUVEC, in accordance with some embodiments.

FIG. 39D shows an example of a bright field microscopy of HUVEC cells in the DAS-COL-STARCH gel, in accordance with some embodiments.

FIG. 39E shows an example of a bright field microscopy of HUVEC cells in the DAS-COL-STARCH gel with a growth factor, in accordance with some embodiments.

FIG. 40A shows an example of mixed human skin collagen type I with DAS and acetic acid before cross-linking, in accordance with some embodiments.

FIG. 40B shows an example of cross-linked human derived collagen and DAS-based material, in accordance with some embodiments.

FIG. 40C shows an example of synovial cell mixed human skin collagen type I gel cross-linked with DAS, in accordance with some embodiments.

FIG. 41A shows an example of human ECM extracts from placenta with DAS and acetic acid before cross-linking, in accordance with some embodiments.

FIG. 41B shows an example of cross-linked human derived ECM and DAS-based material, in accordance with some embodiments.

FIG. 41C shows another example of synovial cell mixed with human ECM extracts gel cross-linked with DAS, in accordance with some embodiments.

FIG. 42 shows a DAS-COL hydrogel with three layers.

FIG. 43 shows a fibrous substance formed as a gel with various compositions of DAS and collagen.

### DETAILED DESCRIPTION OF THE INVENTION

The present application relates generally to compositions, methods, and systems for generating and/or using dialdehyde starch (DAS)-collagen (DAS-COL) matrices. In many cases, compositions disclosed herein (e.g., compositions comprising DAS-COL) have greater wet strength than existing biomatrices, allowing for molding and in situ or patterned bioprinting of matrices.

In some cases, DAS-COL is mixed with cells during or prior to matrix molding or bioprinting. The inclusion of cells in compositions, systems, and methods disclosed herein often improves the success rate of graft or implant procedures, improve the physical and mechanical characteristics of compositions disclosed herein (e.g., through modification and/or deposition of matrix components), and/or improve the durability of compositions disclosed herein.

Further, compositions, systems, and methods disclosed herein often result in improved survival and/or function of cells that are included in molded or bio-printed matrices. For example, methods, compositions, and systems are disclosed herein for modulating the pH of compositions comprising matrix molecules (e.g., DAS-COL) and cells, which, in some cases, improve cell survival during the formation of an implant or bio-printable composition (e.g., a bioink) and/or after application of the implant or bio-printable composition(s) to a subject's body (e.g., a knee joint, a shoulder joint, or a hip joint).

Bioprinting often includes a process of generating spatially-controlled cell patterns using 3D printing technologies, where cell function and viability are preserved within the printed tissue construct. Bio-printing typically involves dispensing cells onto a biocompatible scaffold using a successive layer-by-layer approach to generate tissue-like three dimensional structures. Such constructed tissue like 3D material is often then implanted into the patient's body.

More recently, attention has been drawn to less invasive, yet efficient artificial tissue implant procedures, using direct bioprinting. Direct bioprinting, in some cases, provides various advantages including i) eliminating the need for prior manufacturing, storage, or transportation of pre-printed tissue construct; ii) providing the ability to customize the engineered tissue to perfectly fit defects of any shape or size of tissues; iii) providing the ability to vary the type or amount of tissue being generated during surgery; iv) providing the ability to combine artificial and natural scaffolds as well as living cells; and/or v) enabling direct integration of the newly printed tissue into the host tissue.

### (a) Compositions

An aspect of the disclosure provides a composition comprising a hydrogel and at least one population of cells. In some cases, the hydrogel comprise collagen cross-linked with dialdehyde starch. In some embodiments, the at least one population of cells comprise a plurality of chondrocytes, wherein the at least one population of cells are seeded on the hydrogel. Any suitable types of collagen are contemplated for DAS-COL composition disclosed herein. For example, in some embodiments, the collagen comprises any one of, or a combination of, type I collagen, type II collagen, type III collagen, type IV collagen, type V collagen, type VI collagen, type VII collagen, type VIII collagen, type IX collagen, type X collagen, type XI collagen, type XII collagen, type XIII collagen, type XIV collagen, type XV collagen, type XVI collagen, type XVII collagen, type XVIII collagen, type XIX collagen, type XX collagen, type XXI collagen, type XXII collagen, type XXIII collagen, type XXIV collagen, type XXV collagen, type XXVI collagen, type XXVII collagen, type XXVIII collagen, or type XXIX collagen. In some embodiments, the collagen in the composition comprises Type I collagen, Type II collagen, Type IX collagen, Type X collagen, or Type XI collagen. Alternatively and/or additionally, the collagen in the composition comprises a combination of any of Type I collagen, Type II collagen, Type IX collagen, Type X collagen, or Type XI collagen. In some embodiments, the collagen is a human derived collagen. In some embodiments, the collagen is a human skin collagen (e.g., collagen type I).

In some cases, a composition (e.g., a hydrogel) disclosed herein comprises type I collagen. Type I collagen provides significant tensile strength to a material. When cross-linked (e.g., by a cross-linking agent, such as dialdehyde starch), type I collagen often improves the strength of an implantable or bio-printable matrix (e.g., hydrogel) such that durability of the composition including type I collagen is often improved, for example, when the composition is implanted or printed in a location that is expected to experience frequent or high magnitude forces, such as a joint of a subject. In many cases, type I collagen comprises a triple-helical structure.

In some cases, a collagen molecule is modified prior to incorporation into a composition disclosed herein. For example, multi-helical collagen structure, such as type I collagen, in some cases, is denatured to form a molecule having a single-helical structure (e.g., gelatin). In some cases, a collagen molecule or denatured protein disclosed herein (such as gelatin) is broken down (e.g., digested using an enzyme) to hydrolyzed collagen. Denaturing or digesting a collagen molecule often affects the mechanical properties of a composition (e.g., matrix, for example, a hydrogel) comprising the denatured and/or digested molecule. Thus, in some embodiments, it is contemplated that denaturing or digesting a molecule (e.g., a collagen molecule, such as type I collagen) affects mechanical properties (e.g., mass density, modulus of elasticity, creep relationship (e.g., creep rate), stress relaxation relationship (e.g., stress relaxation rate), tensile modulus (e.g., Young's modulus), and/or ultimate tensile strength) of the composition.

(i) Cross-Linking Agents

Two or more collagen molecules (or any modified forms of collagen (e.g., gelatin, etc.)) of a composition, system, or method disclosed herein are crosslinked by a linker molecule or a cross-linking agent. In some cases, a linker molecule or cross-linking agent or a portion thereof is consumed in the process of joining a first molecule to a second molecule. In some cases, a linker molecule or cross-linking agent or a portion thereof is preserved in the process of joining a first molecule to a second molecule. For example, in some cases, a cross-linking agent (or a portion thereof) forms a portion of a composition disclosed herein after participating in a reaction to join a first matrix molecule and a second matrix molecule.

In many cases, compositions disclosed herein comprise dialdehyde starch (DAS) as a crosslinking agent. In some embodiments, collagen is polymerized into a hydrogel by DAS as a cross-linking agent as shown below:

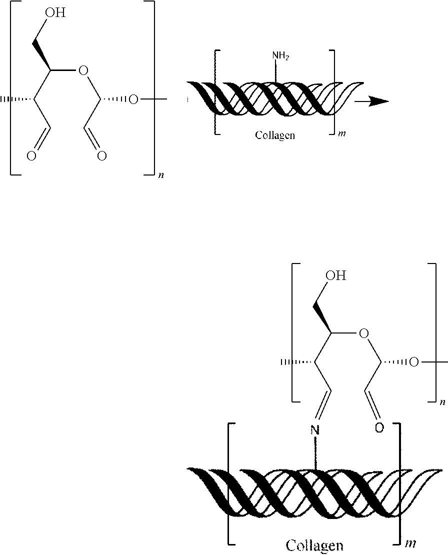

Alternatively and/or additionally, in some cases, the crosslinking agent comprises calcium (Ca²⁺), magnesium (Mg²⁺), calcium chloride, calcium sulfate, calcium carbonate, glutaraldehyde, genipin, nordihydroguaiaretic acid, tannin acid, procyanidin, 1-ethyl-3-3-dimethylaminopropylcarbodiimide hydrochloride (EDC), divinyl benzene (DVB), ethylene glycol dimethacrylate (EGDMA), tetraethylene glycol diacrylate (TEGDA), polyethylene glycol diacrylate (PEGDA), or a combination thereof. In some embodiments, the DAS crosslinker provides an antibacterial property. In some embodiments, DAS is non-cytotoxic.

In some cases, it is contemplated that the concentration, ratio and/or amount of collagen in the composition varies depending on the types of collagen, desired mechanical or chemical properties of the composition, and/or the types or concentration of the crosslinking agents. Thus, for example, the concentration of the collagen in the hydrogel is from 0.1% to 95% weight to volume, from 0.1% to 85% weight to volume, from 0.1% to 75% weight to volume, from 0.1% to 65% weight to volume, from 0.1% to 55% weight to volume, from 0.1% to 45% weight to volume, from 0.1% to 35% weight to volume, from 0.1% to 25% weight to volume, from 0.5% to 90% weight to volume, from 0.5% to 80% weight to volume, from 0.5% to 70% weight to volume, from 0.5% to 60% weight to volume, from 0.5% to 50% weight to volume, from 0.5% to 40% weight to volume, from 0.5% to 30% weight to volume, from 1% to 10% weight to volume, from 1% to 9% weight to volume, from 1% to 8% weight to volume, from 1% to 7% weight to volume, from 2% to 10% weight to volume, from 3% to 10% weight to volume, from 4% to 10% weight to volume, from 4% to 9% weight to volume, or from 4% to 8% weight to volume. In some embodiments, a hydrogel comprising 4% collagen cross linked with 5% DAS is used to support chondrocyte to generate a stiff cartilage-like tissue.

Similarly, in some cases, the concentration of the cross-linking agents in the composition varies depending on the types or concentration of matrix molecules (e.g., collagen, etc.), types of cross-linking agents, desired mechanical or chemical properties of the composition (e.g., rigidity, toxicity, etc.), or cross-linking conditions (e.g., temperature, pH, light (e.g., ultraviolet light), etc.) using the cross-linking agent. In some embodiments, the concentration of the DAS in the DAS-COL composition ranges from 0.01% to 80% weight to volume, 0.01% to 70% weight to volume, 0.01% to 60% weight to volume, 0.01% to 50% weight to volume, 0.01% to 40% weight to volume, 0.01% to 30% weight to volume, from 0.01% to 20% weight to volume, from 0.01% to 15% weight to volume, from 0.01% to 10% weight to volume, from 0.01% to 5% weight to volume, from 1% to 30% weight to volume, from 1% to 20% weight to volume, from 1% to 10% weight to volume, from 2% to 30% weight to volume, from 1% to 20% weight to volume, from 2% to 10% weight to volume, from 5% to 30% weight to volume, from 5% to 20% weight to volume, from 5% to 10% weight to volume, or at a concentration (weight to volume) of about 75%, about 65%, about 55%, about 45%, about 40%, about 35%, about 30%, about 25%, about 20%, about 15%, about 10%, about 5% weight to volume of the composition. In some cases, the cross-linking agent comprises DAS at a concentration between about 0.1% weight to volume to about 0.3% weight to volume. In some cases, the cross-linking agent comprises DAS at a concentration of about 0.25% weight to volume.

In some cases, the ratio between the matrix molecules and the cross-linking agents varies depending on the types of matrix molecules and the cross-linking agents, desired mechanical or chemical properties of the composition (e.g., rigidity, toxicity, etc.), or cross-linking conditions (e.g., temperature, pH, light (e.g., ultraviolet light), etc.). In some embodiments, the ratio between collagen and DAS in the DAS-COL composition ranges from 1:20 to 20:1, 1:15 to 15:1, 1:10 to 10:1, 1:9 to 9:1, 1:8 to 8:1, 1:7 to 7:1, 1:6 to 6:1, 1:5 to 5:1, 1:4 to 4:1, 1:3 to 3:1, 1:2 to 2:1, about 1:1, about 2:1, about 3:1, about 4:1, about 5:1, about 6:1, about 7:1. about 8:1, about 9:1, or about 10:1.

In some embodiments, such generated DAS-COL composition (or hydrogel) forms a viscous gel. In some embodiments, the desired viscosity of the composition varies depending on the use of the composition (e.g., injectable gel, moldable gel, location of the implant or graft, etc.) or any additional molecules or ingredients that is added to the composition after solidification process. In some embodiments, the viscosity of the DAS-COL composition or hydrogel ranges from 10,000 centipoise (cps) to 250,000 cps. In some embodiments, the viscosity of the DAS-COL composition or hydrogel is less than 10,000 cps or more than 250,000 cps. In some embodiments, the viscosity of the DAS-COL composition or hydrogel is about 10,000 cps to about 30,000 cps, about 10,000 cps to about 60,000 cps, about 10,000 cps to about 90,000 cps, about 10,000 cps to about 120,000 cps, about 10,000 cps to about 150,000 cps, about 10,000 cps to about 200,000 cps, about 10,000 cps to about 250,000 cps, about 30,000 cps to about 60,000 cps, about 30,000 cps to about 90,000 cps, about 30,000 cps to about 120,000 cps, about 30,000 cps to about 150,000 cps, about 30,000 cps to about 200,000 cps, about 30,000 cps to about 250,000 cps, about 60,000 cps to about 90,000 cps, about 60,000 cps to about 120,000 cps, about 60,000 cps to about 150,000 cps, about 60,000 cps to about 200,000 cps, about 60,000 cps to about 250,000 cps, about 90,000 cps to about 120,000 cps, about 90,000 cps to about 150,000 cps, about 90,000 cps to about 200,000 cps, about 90,000 cps to about 250,000 cps, about 120,000 cps to about 150,000 cps, about 120,000 cps to about 200,000 cps, about 120,000 cps to about 250,000 cps, about 150,000 cps to about 200,000 cps, about 150,000 cps to about 250,000 cps, or about 200,000 cps to about 250,000 cps. In some embodiments, the viscosity of the DAS-COL composition or hydrogel is about 10,000 cps, about 30,000 cps, about 60,000 cps, about 90,000 cps, about 120,000 cps, about 150,000 cps, about 200,000 cps, or about 250,000 cps.

In some embodiments, the DAS-COL composition has a mechanical stiffness ranging from 1 kPa to 2000 kPa, from 1 kPa to 1000 kPa, from 1 kPa to 500 kPa, from 1 kPa to 250 kPa, from 1 kPa to 100 kPa, from 2 kPa to 1000 kPa, from 2 kPa to 500 kPa, from 2 kPa to 250 kPa, from 5 kPa to 500 kPa. Alternatively, in some cases, the DAS-COL composition or hydrogel has a mechanical stiffness ranging from 1000 kPa to 20 GPa, from 1000 kPa to 10 GPa, from 1000 kPa to 5 GPa, from 1000 kPa to 1 GPa, from 1000 kPa to 100 MPa, 1000 kPa to 10 MPa, 1000 kPa to 5000 kPa, etc. In some embodiments, the DAS-COL composition has a mechanical stiffness ranging from 10 kPa to 1000 kPa.

In some embodiments, the DAS-COL composition has pH, which varies depending on the use of the composition (e.g., use as a bioink, location of the placement of the composition in the subject, etc.), and/or any other biological materials (e.g., cells, etc.) that is further added or mixed in the composition. Thus, for example, the pH of the DAS-COL composition or hydrogel ranging from 3.0 to 11.0, from 3.5 to 10.5, from 4 to 10, from 4.5 to 9.5, from 5 to 9, from 5.5 to 8.5, from 6 to 8, from 6.5 to 7.5, or about 5.5, about 6.0, about 6.5, about 7, about 7.5, about 8, about 8.5, etc. In some embodiments, where the composition includes live cells before the collagen is at least 70%, at least 80%, at least 90% polymerized, the pH of the DAS-COL composition during polymerization and after polymerization does not induce the cell death of at least 20%, at least 30%, at least 40%, at least 50%, at least 60%, at least 70% of the cells initially mixed or seeded in the composition due to the acidic or basic condition of the composition.

(ii) Cells

In some embodiments, the composition includes one or more types of cells. Where the composition includes a plurality of cell types, the cells comprises about 2, about 3, about 4, about 5, about 6, about 7, about 8, about 9, about 10, about 12, about 14, about 16, about 18, about 20, about 25, about 30, about 35, about 40, about 45, about 50, about 55, about 60, about 65, about 70, about 75, about 80, about 85, about 90, about 95 or about 100 cell types. In some embodiments, the composition comprises more than 100 cell types.

In some cases, any suitable types of cells that are viable in the DAS-COL composition or hydrogel are contemplated. For example, in some cases, the types of cells comprise chondrocytes, pluripotent cells, chondrocytes, osteoblasts, synovial cells, mesenchymal stem cells, adipose stromal vascular cells, meniscus cells, infrapatellar fat pad-derived stem cells (IPFP), pericytes, endothelial cells, myoblasts, or any combination thereof. In some embodiments, the plurality of cells comprises chondrocytes, chondroprogenitor cells, keratinocytes, hair root cells, hair shaft cells, hair matrix cells, exocrine secretory epithelial cells, hormone secreting cells, epithelial cells, neural or sensory cells, photoreceptor cells, muscle cells, extracellular matrix cells, blood cells, cardiovascular cells, endothelial cells, vascular smooth muscle cells kidney cells, pancreatic cells, immune cells, stem cells, germ cells, nurse cells, interstitial cells, stellate cells liver cells, gastrointestinal cells, lung cells, tracheal cells, vascular cells, skeletal muscle cells, cardiac cells, skin cells, smooth muscle cells, connective tissue cells, corneal cells, genitourinary cells, breast cells, reproductive cells, endothelial cells, epithelial cells, fibroblasts, Schwann cells, adipose cells, bone cells, bone marrow cells, cartilage cells, pericytes, mesothelial cells, cells derived from endocrine tissue, stromal cells, progenitor cells, lymph cells, endoderm-derived cells, ectoderm-derived cells, mesoderm-derived cells, pericytes, or progenitors thereof and/or a combination thereof. In some embodiments, the plurality of cells comprises chondrocytes. In some embodiments, the plurality of cells comprises chondroblasts. In some embodiments, the plurality of cells comprises mesenchymal stem cells. In some embodiments, the plurality of cells comprises connective tissue fibroblasts, tendon fibroblasts, bone marrow reticular tissue fibroblasts, non-epithelial fibroblasts, pericytes, osteoprogenitor cells, osteoblasts, or osteoclasts or any combination thereof. In some embodiments, the plurality of cells comprises articular chondrocytes. In some embodiments, the plurality of cells is selected from stem cells, progenitor cells, totipotent cells, pluripotent cells, induced pluripotent stem cells, undifferentiated cells, differentiated cells, differentiating cells, trans-differentiating cells, cells from an adult, cells from a child, germ cells, circulating cells, resident cells, adherent cells, malignant cells, tumor cells, proliferating cells, quiescent cells, senescent cells, apoptotic cells, cytokine-producing cells, migrating cells, or a combination thereof.

In some embodiments, the composition comprises a plurality of cells that express cell adhesion molecules. In some embodiments, cell adhesion molecules are selected from one or more of an adherin, a cadherin, a calsyntenin, a claudin, a cluster differentiation protein, a contactin, an immunoglobulin, an integrin, a lectin, a nectin, an occludin, a vinculin, a porimin, a podoplanin, a podocalyxin, a periostin, a neurotrimin, a neurexin, and a selectin. In some embodiments, the cell adhesion molecule is a receptor. In some embodiments, the cell adhesion molecule is a transmembrane protein.

In some embodiments, at least a portion of the plurality of cells comprises a genetic mutation. In some embodiments, some cells comprise a naturally-occurring genetic mutation. In some embodiments, the naturally-occurring genetic mutation is a germline genetic mutation or a somatic genetic mutation. In some embodiments, some cells comprise an induced genetic mutation. In some embodiments, the induced genetic mutation comprises a random genetic mutation or a targeted genetic mutation. In some embodiments, one or more genes in the plurality of cells comprise a genetic mutation. In some embodiments, 2, 3, 4, 5, 6, 7, 8, 9 or 10 genes in the plurality of cells comprise a genetic mutation. In some embodiments, more than 10 genes in the plurality of cells comprise a genetic mutation. In some embodiments, a gene comprises a plurality of genetic mutations. In some embodiments, some cells are genetically modified. In some embodiments, some cells are transfected with a nucleic acid. In some embodiments, some cells are infected by a virus comprising a nucleic acid. In some embodiments, some cells are transduced by a virus comprising a nucleic acid. In some embodiments, the virus is selected from a retrovirus, adenovirus or adeno-associated virus. In some embodiments, the nucleic acid is selected from a vector, a plasmid, a gene, a non-coding nucleic acid, an exon, an intron, a double stranded DNA, a single stranded DNA, a RNA, a siRNA, or a miRNA. In some embodiments, the nucleic acid is a gene. In some embodiments, the gene is a eukaryotic gene. In some embodiments, the gene is a prokaryotic gene. In some embodiments, the nucleic acid encodes a label or an affinity tag.

In some embodiments, at least a portion of the plurality of cells comprises one or more labels. In some embodiments, the one or more labels comprise a fluorescent probe. In some embodiments, the fluorescent probe is selected from a CellTrace™ or CellTracker™ (Life Technologies, Carlsbad, CA, USA). In some embodiments, the label comprises a fluorescent tag. In some embodiments, the fluorescent tag is mPlum, mCherry, tdTomato, mStrawberry, J-Red, DsRed-monomer, mOrange, mKO, mCitrine, Venus, YPet, EYFP, Emerald EGFP, CyPet, mCFPm, Cerulean, T-Sapphire, GFP or YFP. In some embodiments, the one or more labels comprises an affinity tag, which is a peptide (e.g., myc-tag, c-myc tag, FLAG-tag, His-tag, polyhistidine tag, HA-tag, V5, VSVG, softag 1, softag 3, express tag, S tag, fluorescein isothiocyanate (FITC), dinitrophenyl, trinitrophenyl, peridinin chlorophyll protein complex, biotin, phycoerythrin (PE), streptavidin, avidin, horse radish peroxidase (HRP), palmitoylation, nitrosylation, alkaline phosphatase, glucose oxidase, glutathione-S-transferase (GST), SUMO tag, thioredoxin, poly(NANP), poly-Arg, calmodulin binding protein, PurF fragment, ketosteroid isomerase, PaP3.30, TAF12 histone fold domain, maltose binding protein, or a fragment thereof).

In some embodiments, the plurality of cells is from a tissue bank. In some embodiments, the plurality of cells is frozen or previously frozen. In some embodiments, the plurality of cells is harvested or isolated from a donor tissue. In some embodiments, the donor tissue is harvested from a live animal. In some embodiments, the donor tissue is derived from a monkey, an ape, a gorilla, a chimpanzee, a cow, a horse, a dog, a cat, a goat, a sheep, a pig, a rabbit, a chicken, a turkey, a guinea pig, a rat or a mouse. In some embodiments, the donor tissue is synthetic. In some embodiments, the plurality of cells is harvested from a live human donor. In some embodiments, the plurality of cells is derived from the individual. In some embodiments, the plurality of cells comprises Human umbilical vein endothelial cells (HUVECs).

In some embodiments, the donor tissue is harvested from a cadaver. In some embodiments, the plurality of cells is harvested from a cadaver. In some embodiments, wherein the plurality of cells is harvested from a cadaver, the plurality of cells is harvested less than about 1 hour, less than about 2 hours, less than about 4 hours, less than about 6 hours, less than about 12 hours, less than about 24 hours, less than about 36 hours, less than about 48 hours, less than about 72 hours after death. In some embodiments, the plurality of cells is harvested from a cadaver less than about 72 hours after death. In some embodiments, the plurality of cells is harvested from a cadaver between 22 hours and 72 hours after death.

In some embodiments, the plurality of cells is treated with an antibiotic and/or an antimycotic after or while they are isolated or harvested. In some embodiments, the antibiotic comprises penicillin, streptomycin, actinomycin D, ampicillin, blasticidin, carbenicillin, cefotaxime, fosmidomycin, gentamicin, kanamycin, neomycin, polymyxin B, or any combination thereof. In some embodiments, the antimycotic is amphotericin B, nystatin, natamycin or any combination thereof.

In some embodiments, the plurality of cells is propagated or maintained in a cell culture media after they are isolated and before they are mixed with or seeded into the DAS-COL composition. In some embodiments, cell culture media comprises essential nutrients, growth factors, salts, minerals, vitamins, platelet-rich plasma, or a combination thereof. In some embodiments, particular ingredients are selected to enhance cell growth, differentiation, or secretion of specific proteins. In some embodiments, cell culture media comprises cellular differentiation agents. In some embodiments, the plurality of cells is cultured with a supernatant or conditioned media from another population of cells in cell culture. In some embodiments, the plurality of cells is cultured in an atmosphere of about 1%, about 2%, about 3%, about 5%, about 7%, about 10% or about 20% O₂. In some embodiments, cells are cultured in an atmosphere of about 1%, about 2%, about 3%, about 4%, about 5%, about 6%, about 7%, about 8%, about 9% or about 10% CO₂. In some embodiments, cells are cultured at a temperature of about 30° C., about 32° C., about 33° C., about 34° C., about 35° C., about 36° C., about 37° C., about 38° C., about 39° C., about 40° C. or about 42° C. In some embodiments, human chondrocytes are cultured at approximately 37° C. with humidified air containing 5% CO₂, media changed about every four days. In some embodiments, the plurality of cells is used for bioprinting when they grow to about 10%, about 20%, about 30%, about 40%, about 50%, about 60%, about 70%, about 80%, about 90%, about 100% confluence.

In some embodiments, the plurality of cells comprises human chondrocytes, fibrochondrocytes or chondrocyte progenitors. For example, human chondrocytes are cultured (e.g., in a cell culture comprising a growth factor such as TGF-β and FGF-β) until they reach to about 80% to 90% confluence before used for bioprinting. In some embodiments, the chondrocytes are cultured in a three-dimensional cell culture.

In some embodiments, the plurality of cells comprising chondrocytes and chondrogenic precursor cells, are added to a composition described herein as a heterogeneous cell population or as a homogenous population. In such embodiments, markers of chondrogenic precursor cells and chondrocytes are used to identify or isolate chondrogenic precursor cells or chondrocytes, which comprise one or more of cathepsin B, chondrocyte expressed protein-68 (CEP-68), type X collagen, type II collagen, aggrecan, Collagen 9, YKL-39, YKL-40, osteonectin, Sox9, annexin A6, CD44, CD151, type IV collagen, CRTAC1, DSPG3, FoxC1, FoxC2, IBSP/Sialoprotein II, ITM2A, Matrilin-3, Matrilin-4, MIA, OCIL, Otoraplin, SoxS, or URB. In some embodiments where the heterogenous cell population is added to the composition, cells are mixed in a predetermined ratio (e.g., chondrocyte:chondrogenic precursor cells=1:1, 1:2, 1:3, 3:1, 2:1, etc.) using the isolated homogenous cell population.

In some embodiments, disclosed herein is a homogeneous population of cells, wherein the homogeneous population of cells comprise the following characteristics: a) at least 75% of the population of cells is positive for CD73 by FACS analysis; b) at least 75% of the population of cells is positive for CD105 by FACS analysis; c) immunopositive for vimentin; and d) reduced level of Oct3/4 expression.

In some cases, the concentration of cells in the composition varies depending on the projected use of the composition (e.g., types of tissue to substitute, area or volume of the tissue to substitute, etc.), a type of cells (e.g., chondrocytes, stem cells, etc.), or the physical, mechanical, or chemical properties of the composition (e.g., stiffness, viscosity, the ratio of collagen to DAS, pH of the composition, etc.). In some embodiments, the cell density of composition is about 1 cell/pL, about 10 cells/pL, about 100 cells/pL, about 1 cell/nL, about 10 cells/nL, about 100 cells/nL, about 1 cell/pL, about 10 cells/pL, about 100 cells/μL, about 1000 cells/μL, about 10,000 cells cells/μL, about 100,000 cells/μL. In some embodiments, the cell density of the composition is about 2×10⁶ cells/mL, about 3×10⁶ cells/mL, about 4×10⁶ cells/mL, about 5×10⁶ cells/mL, about 6×10⁶ cells/mL, about 7×10⁶ cells/mL, about 8×10⁶ cells/mL, about 9×10⁶ cells/mL, about 10×10⁶ cells/mL, about 15×10⁶ cells/mL, about 20×10⁶ cells/mL, about 25×10⁶ cells/mL, about 30×10⁶ cells/mL, about 35×10⁶ cells/mL, about 40×10⁶ cells/mL, about 45×10⁶ cells/mL, or about 50×10⁶ cells/mL. In some embodiments, the cell density of the composition is about 1 million cells/ml to about 50 million cells/ml.

In some cases, it is contemplated that cells in the composition are distributed in the composition in various manners depending on the types of cells, the mechanical or chemical properties of the composition (e.g., stiffness, viscosity, the ratio of collagen to DAS, pH of the composition, etc.), and/or the use of the composition (e.g., bioink, moldable composition, implant, etc.). For example, in some embodiments, at least 20%, at least 30%, at least 40%, at least 50%, at least 60%, at least 70%, at least 80%, at least 90%, or at least 95% of the cells in the composition are distributed evenly or randomly throughout at least 20%, at least 30%, at least 40%, at least 50%, at least 60%, at least 70%, at least 80%, at least 90%, or at least 95% of the entire volume of the composition. Alternatively and/or additionally, the cells in the composition are preferentially distributed on the surface of the composition. In such embodiments, it is contemplated that at least 50%, at least 60%, at least 70%, at least 80%, at least 90%, or at least 95% of the cells are distributed on the surface or in the near-surface area (e.g., within a depth of at most 10%, at most 20%, at most 30%, at most 40%, or at most 50% of the entire depth or diameter of the composition in a given point or location, etc.). Alternatively and/or additionally, the cells in the composition are preferentially distributed on one side of the composition. In such embodiments, it is contemplated that at least 50%, at least 60%, at least 70%, at least 80%, at least 90%, or at least 95% of the cells are distributed in at most 60%, at most 50%, at most 40%, at most 30%, or at most 20% volume of the composition. In some embodiments, the cells in the composition are distributed in an elevated or decreased concentrations or amounts (e.g., at least 10% increased or decreased, at least 20% increased or decreased, at least 30% increased or decreased, at least 40% increased or decreased, at least 50% increased or decreased, at least 60% increased or decreased, at least 70% increased or decreased, at least 80% increased or decreased, at least 90% increased or decreased, at least twice increased or decreased, etc.) from one given location of the composition to another given location of the composition. Collagen is naturally present in human body and, in some cases, induces less inflammatory reaction compared to other compounds such as, for example, alginate. Some compounds, such as, for example, gelatin that is a denatured collagen comprises smaller chains amino acids and therefore, in some cases, has less desirable mechanical properties than for example, collagen. A biodegradable artificial polymer, in some cases, has adverse reactions when degraded (e.g. PLA often increases local pH). Non-degradable artificial polymers sometimes generate fragments that are undesirable such as, for example, foreign-body reaction. The hydrogel described herein, often does not behave like an artificial polymer.

In some embodiments, where the composition includes two or more types of cells, the cells are distributed in the composition as a mixed population of cells or in a separated manner. For example, in some cases, where the composition includes type A cells and type B cells, type A cells are distributed evenly throughout the composition while type B cells are distributed preferentially on the surface. In another example, where the composition includes type A cells and type B cells, type A cells are distributed in a layer and type B cells are distributed in another layer below, above, or adjacent to the layer of type A cells.

(iii) Culture Media and Biochemical Factors

In some embodiments, the composition comprises cell culture medium or a buffer reagent. In some cases, it is contemplated that the cell culture medium or the buffer reagent contribute to maintain or enhance the cell viability and/or to prevent loss and/or changes in cell properties (e.g., cell division capacity, cell adhesion capacity, cell proliferation capacity, cell excitation capacity, cell secretion capacity, etc.). In some embodiments, cell culture media is selected from Balanced Salts, Dulbecco's Modified Eagle's Medium, Dulbecco's Modified Eagle's Medium/Nutrient F-12 Media, Ham's F-10 Media, Ham's F-12 Media, Minimum Essential Medium Eagle, Medium 199, RPMI-1640 Medium, Ames' Media, BGJb Medium (Fitton-Jackson Modification), Click's Medium, CMRL-1066 Medium, Fischer's Medium, Glascow Minimum Essential Medium (GMEM), Iscove's Modified Dulbecco's Medium (IMDM), L-15 Medium (Leibovitz), McCoy's 5A Modified Medium, NCTC Medium, Swim's S-77 Medium, Waymouth Medium, William's Medium E, or combinations thereof. In some embodiments, the cell culture medium further comprises a biological serum. In some embodiments, the serum is fetal bovine serum, fetal calf serum, fetal goat serum or horse serum. In some embodiments, the biological serum content of the cell culture medium is about 0.5% v/v, about 1% v/v, about 2% v/v, about 5% v/v, about 10% v/v, about 15% v/v, about 20% v/v, about 50% v/v, about 99% v/v, about 100% v/v. In some embodiments, the cell culture medium comprises a buffering agent.

A buffer reagent (e.g., a buffer), in some cases, includes an aqueous buffer. In some embodiments the buffering agent is selected from 2-(N-morpholino)ethanesulfonic acid (MES), 2-[(2-amino-2-oxoethyl)-(carboxymethyl)amino]acetic acid (ADA, also known as N-(2-acetamido)iminodiacetic acid), peperazine-N,N′-bis(2-ethanesulfonic acid) (PIPES, also known as 1,4-Piperazinediethanesulfonic acid), N-(2acetamido)-2-aminoethanesulfonic acid (ACES, also known as 2-(carbamoylmethylamino)ethanesulfonic acid), 2-hydroxy-3-morpholin-4-ylpropane-1-sulfonic acid (MOPSO), 3-(N-morpholino)propanesulfonic acid (MOPS, also known as 3-morpholinopropane-1-sulfonic acid), N,N-bis(2-hydroxyethyl)-2-aminoethanesulfonic acid (BES), 2-[[1,3,-dihydroxy-2-(hydroxymethyl)propan-2-yl]amino]ethanesulfonic acid (TES), (4-(2-hydroxyethyl)-1-piperazineethanesulfonic acid (HEPES), 3-(N,N-bis[2-hydroxyethyl]amino)-2-hydroxypropanesulfonic acid (DIPSO), Acetamidoglycine, 3-[[1,3-dihydroxy-2-(hydroxymethyl)propanyl-2-yl]amino]-2-hydroxypropane-1-sulfonic acid (TAPSO), piperazine-1,4-bis(2-hydroxypropanesulfonic acid) (POPSO), ((2-hydroxyethyl)-piperazine-N-2-hydroxypropanesulfonic acid (HEPPSO), 3-[4-(2-hydroxyethyl)piperazin-1-yl]propane-1-sulfonic acid (HEPPS, also known as EPPS), N-(2-hydroxy-1,1-bis(hydroxymethyl)ethyl)glycine (Tricine), 2-aminoacetamide (Glycinamide), 2(bis(2-hydroxyehtyl)amino) acetic acid (Bicine) or [tris(hydroxymethyl)methylamino]propanesulfonic acid (TAPS, also known as 3-{[1,3-dihydroxy-2-(hydroxymethyl)propan-2-yl]amino}propane-2-sulfonic acid). In some cases, a buffering agent comprises a zwitterion (e.g., a zwitterionic buffer such as MES, ADA, ACES, PIPES, BES, TES, HEPES, MOPS, MOPSO, DIPSO, TAPS, TAPSO, POPSO, HEPPSO, HEPPS, tricene, glycinamide, or bicine). In some cases, a buffer comprises saline.

In some embodiments, it is contemplated that the composition (or hydrogel) comprises additional molecules that sometimes affect the physical, chemical, and/or mechanical properties of the composition (or hydrogel) and sometimes have no effect on these properties. For example, in some cases, the composition (or hydrogel) comprises one or more types of enzymes, biochemical factor, or a small molecule. In some embodiments, the small molecule comprises a salicylic acid, a carboxylic acid, a lipid or fatty acid, a surfactant, a starch, a paraffin, a silica, a glycerol, or a combination thereof. In some embodiments, the lipid or fatty acid comprises palmitic acid, oleic acid, linolenic acid, omega-3 fatty acid or a combination thereof. For example, starch is added to DAS-COL to enhance printability. In some embodiments, to enhance printability or other properties (e.g., chemical or physical properties) of the composition about 1 w/v % to about 3 w/v % collagen, about 0.1 w/v % to about 0.25 w/v % DAS are mixed with about 5 w/v % to about 20 w/v % starch in a ratio of about 8:1:1. In some cases, the ratio of COL:DAS:starch is about 8:1:1, 7:1:2, 7:2:1, 6:3:1, 6:1:3, or any ratio in between two of the ratios mentioned herein. In some embodiments, starch comprises corn, potato, wheat, tapioca starch, or a combination thereof. In some embodiments, the starch comprises a corn starch. In some embodiments, a starch is added to a DAS-COL mix as a starch solution comprising the starch at a weight to volume (w/v %) of about 10% to about 30%. In some embodiments, the starch is added to a DAS-COL mix as a starch solution comprising the starch at about 10 w/v % to about 15 w/v %, about 10 w/v % to about 20 w/v %, about 10 w/v % to about 25 w/v %, about 10 w/v % to about 30 w/v %, about 15 w/v % to about 20 w/v %, about 15 w/v % to about 25 w/v %, about 15 w/v % to about 30 w/v %, about 20 w/v % to about 25 w/v %, about 20 w/v % to about 30 w/v %, or about 25 w/v % to about 30 w/v %. In some embodiments, the starch solution comprises the starch at about 10 w/v %, about 15 w/v %, about 20 w/v %, about 25 w/v %, or about 30 w/v %. In some embodiments the starch solution comprises the starch at about 10 w/v %, about 15 w/v %, about 20 w/v %, about 25 w/v %, or more. In some embodiments, the starch solution comprises the starch at about 30 w/v %, 25 w/v %, 20 w/v %, 15 w/v %, 10 w/v %, or less. The DAS in DAS-COL mix comprises a DAS solution comprising DAS at about 0.3 w/v % to about 6 w/v %, as described herein. The COL in DAS-COL mix comprises a COL solution comprising COL at about 2 w/v % to about 8 w/v %, as described herein.

In some embodiments, a DAS-COL mix to form a gel substance comprises a solution of DAS comprising DAS at a weight to volume (w/v %) of about 0.3 w/v % to about 0.6 w/v %. In some embodiments, the gel substance is used for bio-printing or injection. In some embodiments, a gel substance is formed from the DAS-COL mix using DAS at about 0.3 w/v % to about 0.4 w/v %, about 0.3 w/v % to about 0.5 w/v %, about 0.3 w/v % to about 0.6 w/v %, about 0.3 w/v % to about 0.7 w/v %, about 0.4 w/v % to about 0.5 w/v %, about 0.4 w/v % to about 0.6 w/v %, about 0.4 w/v % to about 0.7 w/v %, about 0.5 w/v % to about 0.6 w/v %, about 0.5 w/v % to about 0.7 w/v %, or about 0.6 w/v % to about 0.7 w/v %. In some embodiments, a gel substance is formed from the DAS-COL mix using a solution of DAS comprising DAS at about 0.3 w/v %, about 0.4 w/v %, about 0.5 w/v %, about 0.6 w/v %, or about 0.7 w/v %. In some embodiments, a gel substance is formed from the DAS-COL mix using a solution of DAS comprising DAS at no less than about 0.3 w/v %, about 0.4 w/v %, about 0.5 w/v %, or about 0.6 w/v %. In some embodiments, a gel substance is formed from the DAS-COL mix comprising a solution of DAS comprising DAS at no more than about 0.6 w/v %, about 0.5 w/v %, about 0.4 w/v %, about 0.3 w/v %. In some embodiments, a gel substance is made from DAS-COL mix comprising a solution of DAS comprising DAS at a w/v % of more than 0.6 w/v % or less than 0.3 w/v %. In some embodiments, the DAS-COL mix to form a gel substance comprises a solution of COL comprising COL at a weight to volume (w/v %) of about 2 w/v % to about 6 w/v %. In some embodiments, the DAS-COL mix to form a gel substance comprises a solution of COL comprising COL at a weight to volume (w/v %) of less than 2 w/v % or more than 6 w/v %. In some embodiments, the gel is formed by mixing DAS (e.g., a DAS solution at concentrations mentioned herein) and COL (e.g., a COL solution at concentrations mentioned herein) at a ratio of 9:1, 8:2, 7:3, or a ration between any two ratios mentioned herein.

In some cases, a DAS-COL mix to form a porous sponge substance comprises a solution of DAS comprising DAS at a weight to volume (w/v %) of about 3 w/v % to about 6 w/v %. The porous sponge substance is used for making a porous block (e.g., by freeze drying) or a rigid block (e.g., by gel molding). In some embodiments, a porous sponge substance is formed using a solution of DAS comprising DAS at about 3 w/v % to about 3.5 w/v %, about 3 w/v % to about 4 w/v %, about 3 w/v % to about 4.5 w/v %, about 3 w/v % to about 5 w/v %, about 3 w/v % to about 5.5 w/v %, about 3 w/v % to about 6 w/v %, about 3.5 w/v % to about 4 w/v %, about 3.5 w/v % to about 4.5 w/v %, about 3.5 w/v % to about 5 w/v %, about 3.5 w/v % to about 5.5 w/v %, about 3.5 w/v % to about 6 w/v %, about 4 w/v % to about 4.5 w/v %, about 4 w/v % to about 5 w/v %, about 4 w/v % to about 5.5 w/v %, about 4 w/v % to about 6 w/v %, about 4.5 w/v % to about 5 w/v %, about 4.5 w/v % to about 5.5 w/v %, about 4.5 w/v % to about 6 w/v %, about 5 w/v % to about 5.5 w/v %, about 5 w/v % to about 6 w/v %, or about 5.5 w/v % to about 6 w/v %. In some embodiments, a porous sponge substance is formed using a solution of DAS comprising DAS at about 3 w/v %, about 3.5 w/v %, about 4 w/v %, about 4.5 w/v %, about 5 w/v %, about 5.5 w/v %, or about 6 w/v %. In some embodiments, a porous sponge substance is formed using a solution of DAS comprising DAS at no less than about 3 w/v %, about 3.5 w/v %, about 4 w/v %, about 4.5 w/v %, about 5 w/v %, or about 5.5 w/v %. In some embodiments, a porous sponge substance is formed using a solution of DAS comprising DAS at no more than about 6 w/v %, about 5 w/v %, about 4.5 w/v %, about 4 w/v %, about 3.5 w/v %, or about 3 w/v %. In some embodiments, a porous sponge substance is made using a solution of DAS comprising DAS at a w/v % of more than 6% or less than 3%. In some embodiments, the DAS-COL mix to form a gel substance comprises a solution of COL comprising COL at a weight to volume (w/v %) of about 2 w/v % to about 8 w/v %. %. In some embodiments, the DAS-COL mix to form a gel substance comprises a solution of COL comprising COL at a weight to volume (w/v %) of less than 2 w/v % or more than 8 w/v %. In some embodiments, the porous sponge is formed by mixing DAS (e.g., a DAS solution at concentrations mentioned herein) and COL (e.g., a COL solution at concentrations mentioned herein) at a ratio of 9:1, 8:2, 7:3, or a ration between any two ratios mentioned herein.

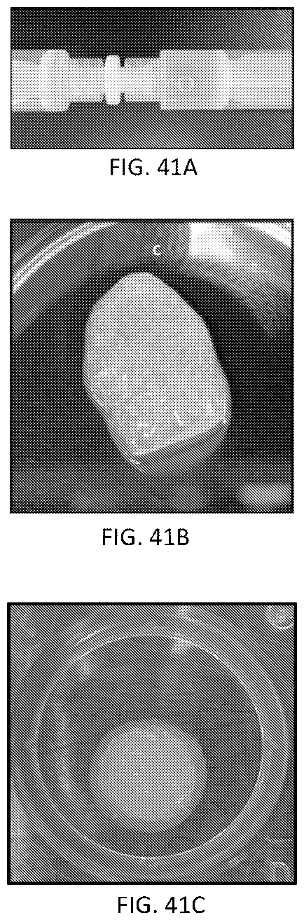

In some embodiments, the composition comprises extracellular matrix (ECM) material (e.g., tissue derived ECM extracts). For example, in some cases, human extracellular matrix (ECM) extracts are obtained from different tissues (e.g., from placenta). In an exemplary embodiment, extracellular matrix (ECM) is mixed with DAS and acetic acid (FIG. 41A). In an exemplary embodiment, the ECM and DAS mixture is cross-linked to form cross-linked DAS and ECM.

In some embodiments, synovial cells are mixed with a gel comprising the ECM and DAS (e.g., crosslinked ECM-DAS or ECM-DAS mix). In an exemplary embodiment, FIG. 41C shows an example of an arbitrary shape formed from synovial cell mixed with human ECM extracts gel cross-linked with DAS. In some embodiments, the synovial cells are mixed with ECM-DAS mix at a concentration of between about 5 million cells/ml to about 50 million cells/ml. In some embodiments, the synovial cells are mixed with DAS-ECM mix at a concentration above 50 million cells/ml or below 2 million cells/ml. In some embodiments, the synovial cells are mixed with the ECM-DAS mix at a concentration of about 25 million cells/ml.

In some embodiments, the biochemical factor is selected from an anticoagulant, albumin, selenium, an amino acid, a vitamin, a hormone, a mineral, or any combination thereof. In some embodiments, the composition comprises a protein. In some embodiments, the protein is a kinase, a hormone, a growth factor, a cytokine, a chemokine, an anti-inflammatory factor, a pro-inflammatory factor, an apoptotic factor or a steroid. In some embodiments, the composition comprises an enzyme. For example, in some embodiments, the composition comprises one or more growth factor (e.g., at least three different types of growth factors, at least four different types of growth factors, or at least five different types of growth factors, two different types of growth factors, three different types of growth factors, four different types of growth factors, or five different types of growth factors, etc.) that is selected from Adrenomedullin (AM), Angiopoietin (Ang), Autocrine motility factor, Bone morphogenetic proteins (BMPs), Brain-derived neurotrophic factor (BDNF), Colony-stimulating factor (CSF), Epidermal growth factor (EGF), Erythropoietin (EPO), Fibroblast growth factor (FGF), Glial cell line-derived neurotrophic factor (GDNF), Granulocyte colony-stimulating factor (G-CSF), Granulocyte macrophage colony-stimulating factor (GM-CSF), Growth differentiation factor-9 (GDF9), Hepatocyte growth factor (HGF), Hepatoma-derived growth factor (HDGF), insulin, Insulin-like growth factor (IGF), Migration-stimulating factor, Myostatin (GDF-8), Nerve growth factor (NGF) and other neurotrophins, Platelet-derived growth factor (PDGF), Thrombopoietin (TPO), Transforming growth factor alpha (TGF-a), Transforming growth factor beta (TGF-□), Tumor necrosis factor-alpha (TNF-α), Vascular endothelial growth factor (VEGF), placental growth factor (P1GF), Fetal Bovine Somatotropin (e.g., bST or bovine growth hormone), IL-1, IL-2, IL-3, IL-4, IL-5, IL-6, IL-7 or a combination thereof. In some embodiments, the composition comprises TGF-□1 and FGF2. In some embodiments, the enzyme is a protease, a collagenase, a nuclease, or a combination thereof. In some embodiments, the protease is a serine protease, a threonine protease, a cysteine protease, an aspartate protease, a glutamic acid protease, a metalloprotease, an exopeptidase, an endopeptidase, a trypsin, a chymotrypsin, a pepsin, a papain, an elastase, a carboxypeptidase, an aminopeptidase, a thrombase, a plasmin, a cathepsin, or snake venom.

In some embodiments, the DAS-COL composition (or hydrogel) comprises one or more types of the specialized proteins. In some embodiments, the proteins comprise fibronectin, laminin, fibrinogen, tenascin, thrombospondin, integrin, or a combination thereof. In some embodiments, the glycosaminoglycan comprises a repeating disaccharide unit. In some embodiments, the disaccharide unit comprises a modified sugar and hexuronic acid. In some embodiments, the modified sugar comprises N-acetylgalactosamine (GalNAc), N-acetylglucosamine (GlcNAc), or a combination thereof. In some embodiments, the hexuronic acid comprises glucuronate (GlcA) or iduronate (IdA). In some embodiments, the glycosaminoglycan comprises hyaluronic acid, dermatan sulfate, chondroitin sulfate, heparin, heparin sulfate, and keratin sulfate. In some embodiments, the glycosaminoglycan is linked to core proteins, forming a proteoglycan. In some embodiments, the core proteins are rich in serine (Ser) and threonine (Thr) residues. In some embodiments, the proteoglycan further comprises a tetrasaccharide linker comprising a glucuronic acid (GlcA) residue, two galactose (Gal) residues, and a xylose (Xyl) residue. In some embodiments, the extracellular matrix is derived from a human, a cow, a horse, a sheep, a goat, a chimpanzee, a monkey, a rat, a pig, a mouse, a rabbit, or a synthetic reaction.

Alternatively and/or additionally, in some cases, the DAS-COL composition comprises one or more types of synthetic or natural polymer or a combination thereof. In some embodiments, the composition is a gel. In some embodiments, the gel is a bio-gel or a hydrogel. In some embodiments, the synthetic polymer is polylactide (PLA), polycaprolactone (PCL), polyethylene glycol (PEG), a PEG macromer, polyethylene glycol methacrylate (PEGMA), polyethylene dimethacrylate (PEGDMA), poly(hydroxyethyl methacrylate) (PHEMA), polyvinyl alcohol (PVA), polyvinylpyrrolidone (PVP), carboxymethyl cellulose (CMC), polyimide (PI), polyacrylate (PAA), polyurethane (PU), PEG-lactide, PEG-glycolide or a combination thereof. In some embodiments, the gel comprises a PEGDMA hydrogel. In some embodiments, the PEGDMA polymer is 10% w/v hydrogel. In some embodiments, the PEGDMA polymer is 20% w/v hydrogel. In some embodiments, the gel does not comprise a synthetic polymer. In some embodiments, PEG macromers comprise reactive chain ends such as acrylate, methacrylate, allyl ether, maleimide, vinyl sulfone, NHS ester and vinyl ether groups. In some embodiments, the alcohol chain ends of PEG are esterified using acid chlorides (e.g., acryloyl chloride, methacryloyl chloride) in the presence of base. In some embodiments, PEG chain ends are etherified under basic conditions by reaction with alkyl halides such as 2-chloroethyl vinyl ether or allyl bromide. In some embodiments, acrylate, methacrylate, vinyl sulfone, maleimide, vinyl ether and allyl ether are capable of step growth network formation or polymerization. In some embodiments, polymerization of macromers is initiated using redox-generated radicals (e.g., ammonium persulfate and TEMED), or radicals generated with light. In some embodiments, the natural polymer is alginate, cellulose, gelatin, pectin, chitosan, paraffin, agarose, or a combination thereof. In some embodiments, the composition comprises Matrigel®.

In some embodiments, where the composition include gelatin, the amount and/or concentration of gelatin varies depending on the desired mechanical or chemical properties of the composition. For example, in some cases, the ratio of gelatin to collagen (gelatin:collagen) ranges from 1:10 to 10:1, from 1:9 to 9:1, from 1:8 to 8:1, from 1:7 to 7:1, from 1:6 to 6:1, from 1:5 to 5:1, from 1:4 to 4:1, from 1:3 to 3:1, from 1:2 to 2:1, from 1:1 to 1:10, from 1:1 to 1:20, from 1:1 to 1:30, from 1:1 to 1:40, from 1:1 to 1:2, from 1:1 to 1:3, from 1:1 to 1:5. In some embodiments, the concentration of the gelatin in the composition ranges from 0.1% to 95% weight to volume, from 0.1% to 85% weight to volume, from 0.1% to 75% weight to volume, from 0.1% to 65% weight to volume, from 0.1% to 55% weight to volume, from 0.1% to 45% weight to volume, from 0.1% to 35% weight to volume, from 0.1% to 25% weight to volume, from 0.5% to 90% weight to volume, from 0.5% to 80% weight to volume, from 0.5% to 70% weight to volume, from 0.5% to 60% weight to volume, from 0.5% to 50% weight to volume, from 0.5% to 40% weight to volume, from 0.5% to 30% weight to volume, from 1% to 10% weight to volume, from 1% to 9% weight to volume, from 1% to 8% weight to volume, from 1% to 7% weight to volume, from 2% to 10% weight to volume, from 3% to 10% weight to volume, from 4% to 10% weight to volume, from 4% to 9% weight to volume, or from 4% to 8% weight to volume. In some embodiments, the composition comprises gelatin at about 2 time to 3 times of the amount of collagen. In some embodiments, the composition comprises gelatin at about 6 w/v % to about 8 w/v %. In some embodiments, the composition comprises gelatin at more than 8 w/v % or less than 6 w/v %.

In some embodiments, the composition comprises an extracellular matrix molecule. In some embodiments, the extracellular matrix molecule comprises a structural protein, a specialized protein, a glycosaminoglycan (GAG), a proteoglycan, or a combination thereof. In some embodiments, a structural protein comprises collagen, elastin, and fibrillin. In some embodiments, the matrix gel material comprises at least one proteoglycan. In some embodiments, the proteoglycan is composed of a core protein with pending glycosaminoglycan (GAG) molecules. In some cases, suitable GAGs comprise hyaluronic acid, chondroitin-4-sulfate, chondroitin-6-sulphate, dermatan sulphate, heparin sulphate, and keratan sulphate. In some embodiments a GAG molecule is linked to the core protein via a trisaccharide linker (e.g. a GalGalXyl-linker). Exemplary proteoglycans include, but are not limited to, decorin, biglycan, versican, and aggrecan. In some embodiments, the proteoglycans are interconnected by hyaluronic acid molecules. In some embodiments, multiple proteoglycans are attached to a single hyaluronic acid backbone. In some embodiments, the ratio of collagen to proteoglycan is in the range of about 0.3 to about 1.1 relative to about 1 of collagen in weight ratio. In some embodiments, the proteoglycan is in the range of about 0.5 to about 0.7 relative to about 1 of collagen.

In some embodiments, such extracellular matrix molecules are present in the composition as a separate/distinct molecule (e.g., without a chemical bond with other molecules in the composition), or as being conjugated with one or more molecules in the composition. For example, in some cases, where the composition includes heparin, heparin is present as a separate molecule in the composition. Alternatively, in some cases, heparin is conjugated with collagen and/or gelatin, via a linker (e.g., a peptide linker, a glycine-rich linker, etc.). In such embodiments, a collagen or gelatin molecule are conjugated with a single heparin molecule. In other embodiments, a collagen or gelatin molecule are conjugated with a plurality of heparin molecules (e.g., multiple heparin molecules conjugated with a single gelatin molecule linearly or via a multi-arm linker).

In some embodiments, the synthetic polymer or the natural polymer comprises a modification to enable crosslinking. In some embodiments, the modification to enable crosslinking is methacrylation. In some embodiments, the synthetic polymer or the natural polymer comprises a functional molecule. In some embodiments, the functional molecule comprises a bioactive protein or drug. In some embodiments, the synthetic polymer or the natural polymer comprises a peptide to promote cell adhesion, a peptide to promote proliferation, or a peptide to promote differentiation. In some embodiments, the peptide to promote cell adhesion is arginyl-glycyl-aspartic acid (RGD). In some embodiments, the synthetic polymer or the natural polymer comprises a biodegradable link. In some embodiments, the biodegradable link is a matrix metalloproteinase (MMP)-sensitive link or an Aggrecanase-sensitive link.

Alternatively and/or additionally, in some cases, the composition comprises one or more types of therapeutic agents. In some embodiments, the therapeutic agent is selected from an antibiotic and/or an antimycotic. In some embodiments, the antibiotic is penicillin, streptomycin, actinomycin D, ampicillin, blasticidin, carbenicillin, cefotaxime, fosmidomycin, gentamicin, kanamycin, neomycin, polymyxin B, or a combination thereof. In some embodiments, the antimycotic is amphotericin B, nystatin, natamycin or a combination thereof. In some embodiments, the therapeutic agent is selected from an anti-inflammatory therapeutic agent. In some embodiments, the anti-inflammatory therapeutic agent is a non-steroidal anti-inflammatory therapeutic agent. In some embodiments, the non-steroidal anti-inflammatory therapeutic agent is a cyclooxygenase (COX) inhibitor. In some embodiments, the COX inhibitor is selected from a COX1 inhibitor, COX2 inhibitor or combination thereof. In some embodiments, the anti-inflammatory therapeutic agent comprises a steroid. In some embodiments, the steroid is a glucocorticoid. In some embodiments, the glucocorticoid is dexamethasone.

Alternatively and/or additionally, in some cases, the composition comprises one or more types of a photoinitiators. In some embodiments, a photoinitiator includes any of type I photoinitiator or a type II photoinitiator. Examples of photoinitiators include, but are not limited to, (2-hydroxy-1-[4-(2-hydroxyethoxy) phenyl]-2-methyl-1-propanone; lithium phenyl-2,4,6-trimethylbenzoylphosphinate (LAP); (2,2′-azobis[2-methyl-N-(2-hydroxyethyl)propionamide]; 2-isocyanotoethyl methacrylate; benzoyl benzylamine; camphorquinone; thiol-norbornene (thiol-ene); riboflavin; lucirin-TPO; Rose Bengal/furfuryl; ethyl eosin; methacrylic anhydride; 2,2-dimethoxy-2-phenylacetophenone; and Eosin Y. In some embodiments, the photoinitiator is added at a final concentration of about 0.01%, 0.02%, 0.03%, 0.04%, 0.05%, 0.1%, 0.15%, 0.2%, 0.25%, 0.3%, 0.35%, 0.4%, 0.45%, 0.5%, 0.6%, 0.7%, 0.8%, 0.9%, or about 1% w/v gel. In some embodiments, the photoinitiator is added at a final concentration of about 0.05% w/v gel.

(iv) Composition Formats

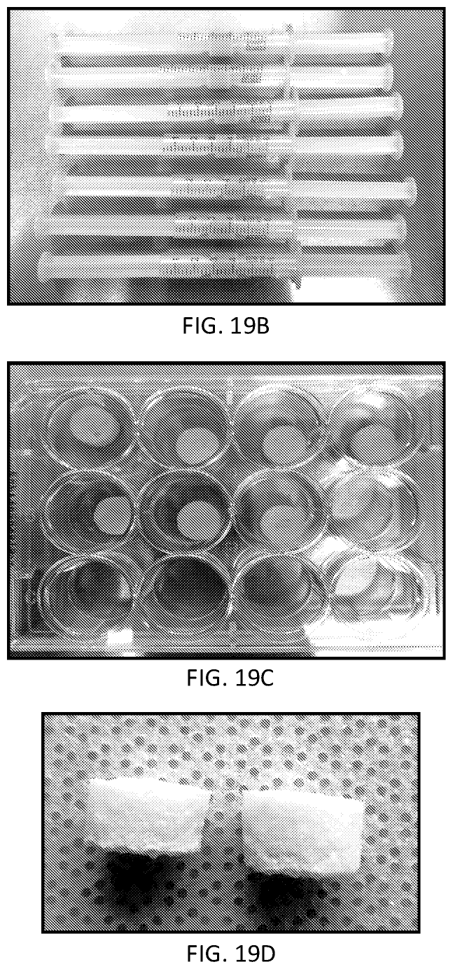

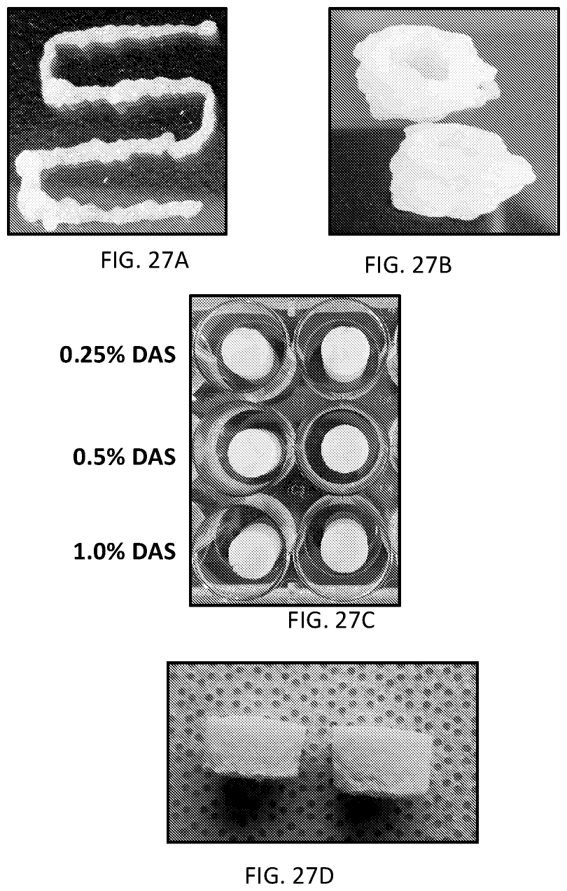

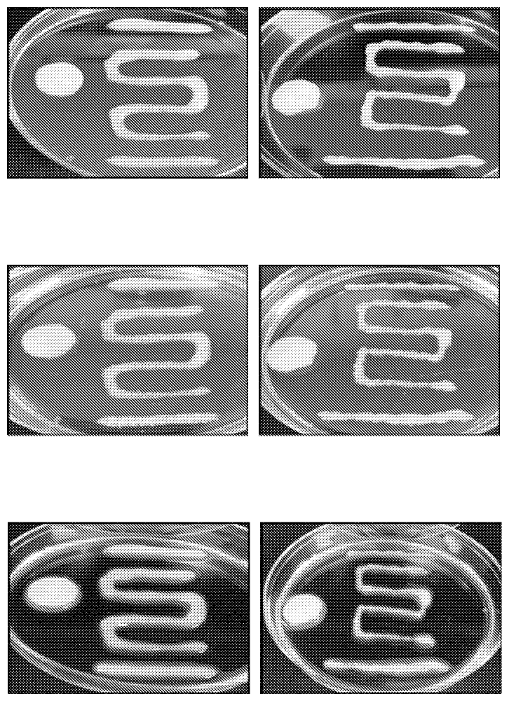

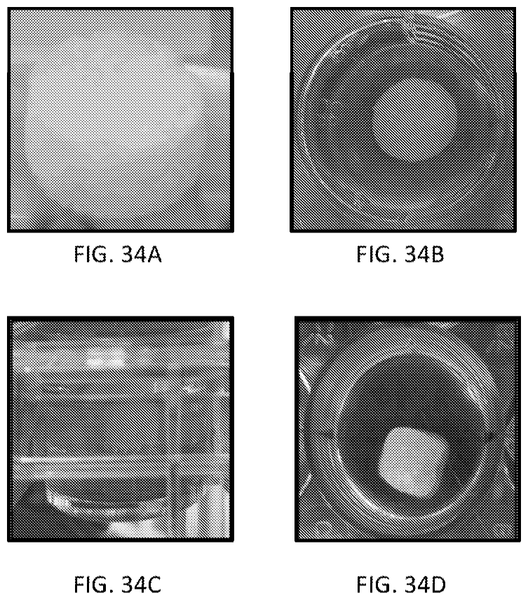

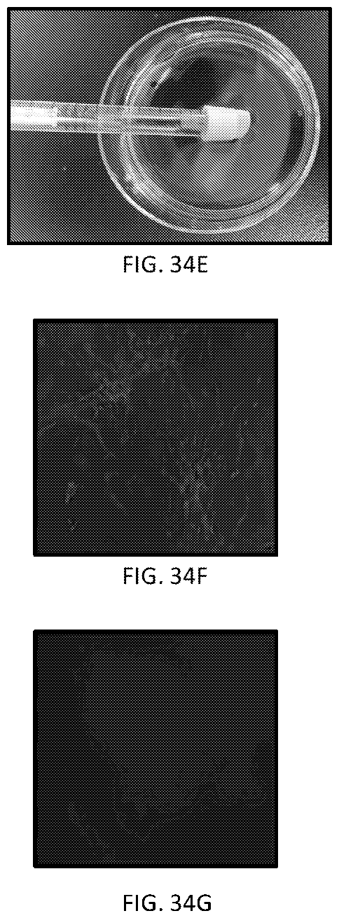

In some embodiments, the composition described herein is generated for various uses. For example, in some cases, the composition is used as a bioink for three-dimensional (3D) printing of a complex tissue or a portion of the tissue. In some cases, the composition used as a bioink includes one or more types of cells that are naturally or preferably present in a tissue to be printed or that help the printed tissue to be implanted and maintained in the subject. Also, in some embodiments, the composition used as a bioink is not completely polymerized or does not achieve full mechanical stiffness before printing such that the composition is shaped via printing without breaking cross-liking bonds between collagen and DAS. In another example, the composition is placed in a mold to shape the composition into a complex tissue or a portion of a tissue. Thus, the shape of the mold mimics the complex tissue or a portion of the tissue including, but not limited to a cartilage, a gum, a bone, at least a portion of a meniscus, at least a portion of an articular cartilage, at least a portion of a rib, at least a portion of an ear, at least a portion of a nose, at least a portion of a bronchial tube, at least a portion of an intervertebral disc, at least a portion of a maxillofacial cartilage, at least a portion of a temporomandibular joint (TMJ), at least a portion of a glenoid labrum, and at least a portion of an acetabular labrum, a cylinder, a cube, a three-dimensional rectangle, a sphere, a crescent, and a tetrahedron. In an exemplary embodiment, molded DAS-COL hydrogels is lyophilized into DAS-COL sponges for storage and/or transportation (FIG. 19D). In some cases, an arbitrary shape is generated using extrusion or bioprinting. In some cases, a shape made from DAS-COL (e.g., molded, extruded or arbitrary) is lyophilized into DAS-COL sponges for storage and/or transportation. In an exemplary embodiment, FIG. 27D shows examples of lyophilized sponges comprising 4 w/v % collagen and 1.0 w/v % dialdehyde starch. In an exemplary embodiment, FIG. 34A shows another example of a lyophilized DAS-COL sponge, generated by mixing 4 w/v % collagen and 0.1 w/v % DAS at a ratio of 9:1. In some embodiments, the composition comprises two or more different layers of hydrogels. In some embodiments, two layers of hydrogels differ by their concentration or amount of collagen and/or DAS, the ratio between collagen and DAS, conditions of polymerization or crosslinking (e.g., temperature, time, pH, etc.) such that the layers have different mechanical and/or chemical properties. In some embodiments, two layers of hydrogels differ by their content including, but not limited to presence or absence of cells, types of cells, concentrations or amounts of cells, presence or absence of, or concentration or amounts of extracellular matrix molecules, natural or synthetic polymers, gelatin, growth factors, cytokines, proteins, enzymes, heparin, therapeutic agents, photoinitiators, buffering agents, serum, medium, etc., such that the layers have different chemical and/or biological properties.

In some embodiments, two layers of hydrogels differ by their molecular structures. For example, in some embodiments, the first layer comprises collagen molecules oriented in a predefined direction A. In such embodiments, it is contemplated that at least 40%, at least 50%, at least 60%, at least 70%, at least 80%, at least 90%, or at least 95% of collagen molecules of the first layer are oriented at an angle of 30 degrees or less relative to the predefined direction A (direction relative to gravity direction, relative to an arbitrary line or plane, relative to traverse, sagittal, lateral, or medial plane of a shape of tissue or portions thereof that the layers of hydrogel is or will be shaping, etc.). Additionally, in some embodiments, at least 40%, at least 50%, at least 60%, at least 70%, at least 80%, at least 90%, or at least 95% of collagen molecules of the second layer are oriented at an angle of 30 degrees or less relative to the predefined direction B that are different from direction A by at least 10 degree, at least 20 degree, at least 30 degree, at least 40 degree, at least 50 degree, at least 60 degree, at least 70 degree, at least 80 degree, at least 90 degree, or perpendicular to the direction A, etc.

Consequently, in some cases, it is also contemplated that the layer of hydrogel with directionally organized collagen molecules are mechanically anisotropic (along the direction or opposite of the direction, etc.) or isotropic. In addition, in some cases, when the two layers are adjacently placed, the combination of two layers provide mechanically anisotropic characteristic or isotropic characteristics even if each layer does not show mechanically anisotropic characteristic or isotropic characteristics in its own. Alternatively, in some cases, when each layer shows mechanically anisotropic characteristic or isotropic characteristics, the combination of two layers do not show either of anisotropic characteristic or isotropic characteristic (by canceling out the directional effects).

### (b) Methods of Generating DAS-COL

Another aspect of the disclosure provides a method of forming a hydrogel by mixing collagen with dialdehyde starch with a population of cells. The amount, concentration, or ratio of collagen, DAS, a population of cells, and/or other optional molecules (e.g., growth factors, cytokines, proteins, medium, buffering agent, etc.) to be used to in the method are as described above.

Any suitable methods for mixing the collagen and DAS, and optionally cells, are contemplated. In some embodiments, collagen and DAS are first mixed in a predetermined condition (e.g., temperature, time duration, pH, etc.) to obtain a desired mechanical and/or chemical property of a hydrogel. In some embodiments, the desired mechanical and/or chemical property of a hydrogel vary depending on the use of the hydrogel. For example, more elasticity or less stiffness would be desired if the hydrogel will be used as a tissue substitute of relatively soft tissue (e.g., cartilage), while more stiffness or less elasticity would be desired if the hydrogel will be used as a tissue substitute of hard tissue (e.g., bone). In some cases, less stiffness and less chemical harshness (or closer to in vivo chemical properties of the subject) would be desired if cells are pre-mixed with the hydrogel such that the hydrogel would be polymerized with the cells inside of the hydrogel.

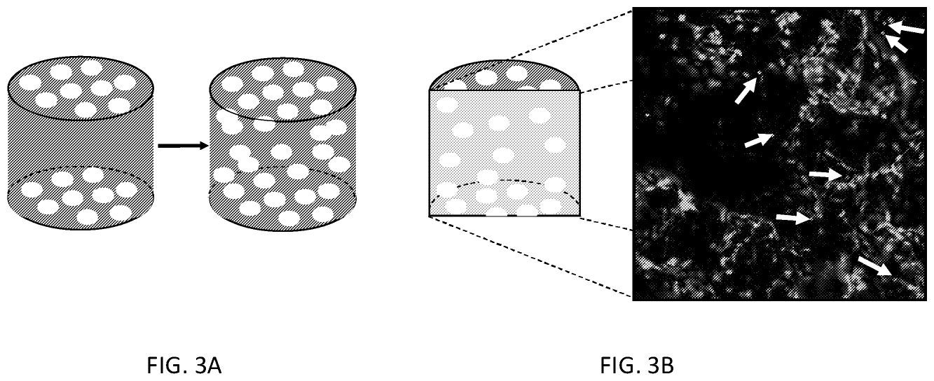

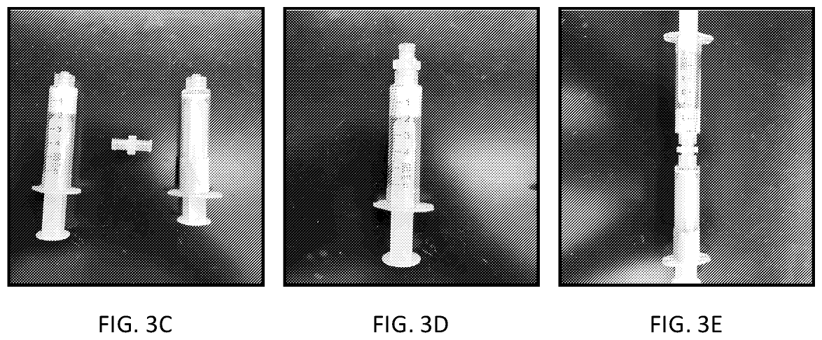

In some cases, DAS and COL are mixed using a mixing system comprising two or more containers (e.g., syringes) and a connection capable of connecting the two or more containers (e.g., syringes). For example, a first solution comprising DAS (e.g., DAS dissolved in water) is placed in a first syringe or container (e.g., as shown in FIGS. 3C-3D). In some cases, a solution comprising collagen (e.g., purified collagen dissolved in water) is placed in a second syringe or container. In some cases, DAS and collagen are mixed by connecting the first syringe to the second syringe with a syringe connector (e.g., male Luer-to-female Luer adapter, Cole-Palmer). In an exemplary embodiment, as shown in FIGS. 3C-3E, the two solutions are mixed by injecting the contents of one syringe into the other syringe, for example, by depressing the plunger of the first syringe and allowing the DAS to flow into the second syringe containing the collagen (e.g., as shown in the top panel of FIG. 3F). In some cases, improved mixing is achieved by subsequently depressing the plunger of the second syringe, causing the mixture of DAS and collagen to flow into the first syringe (e.g., as shown in the middle panel of FIG. 3F). In some cases, further mixing is achieved by repeating the reversal of flow in the syringes described above one or more times (e.g., as shown in the bottom panel of FIG. 3F). In some embodiments, mixing is performed at a steady, moderate rate to avoid introduction of air bubbles into the composition and is continued until the composition is substantially homogeneous (e.g., by visual reference).

In some cases, the predetermined condition also varies depending on the desired mechanical and/or chemical property of a hydrogel. For example, in some cases, the predetermined condition is a temperature of the mixture (at the time of mixing, during the mixing, etc.) that ranges between 10° C.-50° C., between 15° C.-45° C., between 15° C.-40° C., between 15° C.-35° C., between 15° C.-30° C., between 15° C.-25° C., about 20° C., about 25° C., or about 30° C. In some cases, the predetermined condition is a pH of the mixture (at the time of mixing, during the mixing, etc.) that ranges between 3.5 to 10.5, between 4 to 10, between 4.5 to 9.5, between 5 to 9, between 5.5 to 8.5, between 6 to 8, between 6.5 to 7.5, about 6, about 6.5, about 7, about 7.5, about 8, etc. Alternatively and/or additionally, in some cases, the predetermined condition is a time duration that the mixture is incubated after mixing, which ranges between 10 min to 24 hours, between 30 min to 12 hours, between 30 min to 6 hours, between 30 min to 3 hours, between 1 hour to 3 hours, between 1 hour to 6 hours, about 30 min, about 1 hour, about 2 hours, about 3 hours, about 4 hours, about 5 hours, about 6 hours, about 12 hours, etc.

In some embodiments, collagen in the composition is polymerized or crosslinked by photoactivation. For example, in some cases, the collagen is modified to tether with PEG-monoacrylate, which sometimes further reacts with crosslinking agent (e.g., poly(ethylene glycol) diacrylate) in the presence of photoinitiator (e.g., any type I photoinitiator or a type II photoinitiator, Irgacure 2959, 2,2′-azobis[2-methyl-n-(2-hydroxyethyl) propionamide] (VA-086), etc.). Examples of photoinitiators include, but are not limited to, (2-hydroxy-1-[4-(2-hydroxyethoxy) phenyl]-2-methyl-1-propanone; lithium phenyl-2,4,6-trimethylbenzoylphosphinate (LAP); (2,2′-azobis[2-methyl-N-(2-hydroxyethyl)propionamide]; 2-isocyanotoethyl methacrylate; benzoyl benzylamine; camphorquinone; thiol-norbornene (thiol-ene); riboflavin; lucirin-TPO; Rose Bengal/furfuryl; ethyl eosin; methacrylic anhydride; 2,2-dimethoxy-2-phenylacetophenone; and Eosin Y. In some embodiments, the photoinitiator is added at a final concentration of about 0.01%, 0.02%, 0.03%, 0.04%, 0.05%, 0.1%, 0.15%, 0.2%, 0.25%, 0.3%, 0.35%, 0.4%, 0.45%, 0.5%, 0.6%, 0.7%, 0.8%, 0.9%, or about 1% w/v gel. In some embodiments, the photoinitiator is added at a final concentration of about 0.05% w/v gel. In another example, collagen and/or DAS is placed in a photodegradable molecular cage that is degraded either by direct exposure to the light or by activation of a photoactivator. In such embodiments, the collagen-DAS mixture is placed near the light source for desired time durations (e.g., 10 sec, 20 sec, 30 sec, 60 sec, 2 min, 5 min, 10 min, 20 min, 30 min, etc.) until the crosslinking reaction or polymerization reaction is initiated. Any suitable light sources are contemplated, and the exemplary light source includes a laser or ultraviolet light, visible light, etc. In some embodiments, the light source emits light in a focused region. In some embodiments, the light source emits light in a pattern. In some embodiments, the pattern of light cross-links the composition. In some embodiments, the method further comprises a wash step to remove the composition which was not cross-linked. In some embodiments, the light source is connected to the endoscope. In some embodiments, the light source emits light with a visible wavelength of to 400 nm to 700 nm. In some embodiments, the light source emits UV light. In some embodiments, UV light comprises UV-A light, UV-B light, or UV-C light. In some embodiments, UV-A light comprises a wavelength of light between 315 nm and 400 nm. In some embodiments, UV-B light comprises a wavelength of light between 280 nm and 315 nm. In some embodiment, UV-C light comprises a wavelength of light between 100 nm to 280 nm. In some embodiments, the light source is an LED.

In some embodiments, the hydrogel comprises photo-releasable factors. In some embodiments, photo-releasable factors are selected from cells, growth factors, proteases, ligands, hormones, extracellular matrix, cytokines, anti-inflammatory factors, pro-inflammatory factors, adhesion molecules, or a combination thereof. In some embodiments, photo-releasable factors are used to form a feature of the bio-printed tissue (e.g. vasculature). In some embodiments, the composition comprises a PEG with a degradable ester linkage. In some embodiments, the composition comprises a factor that is attached to a component of the composition or the extracellular matrix. In some embodiments, the factor is released by hydrolysis or enzymolysis of a bond that attaches the factor to the component of the gel or extracellular matrix. In some embodiments, the factor is released by hydrolysis or enzymolysis of the gel component or the extracellular matrix. In some embodiments, the factor is released from the gel component or the extracellular matrix by the enzyme. In some embodiments, the enzyme is present in the internal tissue defect. In some embodiments, the factor released is a therapeutic agent or a growth factor. In some embodiments, the growth factor induces angiogenesis upon release.

In some embodiments, a population of cells is premixed with the collagen and/or DAS before crosslinking reaction (polymerization reaction) occurs between the collagen and DAS. Thus, in some embodiments, a population of cells is premixed with the collagen, then DAS is added to the collagen-cell mixture. In some embodiments, a population of cells is premixed with DAS, and the cell-DAS mixture is added to the collagen. In some embodiments, a population of is be added shortly after the collagen and DAS are mixed such that cells are further mixed with collagen-DAS mixture before the crosslinking reaction (polymerization reaction) occurs.

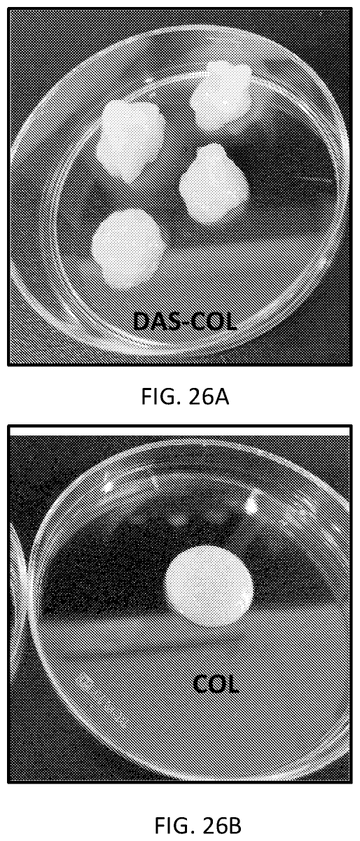

In some embodiments, a population of cells is seeded into the collagen-DAS hydrogel during or after crosslinking reaction (polymerization reaction) occurs. For example, in some embodiments, cells are added to the collagen-DAS mixture within 1 min, within 2 min, within 3 min, within 5 min, within 10 min, or alternatively before at most 20%, at most 30%, at most 40%, at most 50%, at most 60%, at most 70%, at most 80%, at most 90% of crosslinking or polymerization reaction is completed. In some embodiments, the cells are injected into the collagen-DAS mixture such that the cells are distributed evenly or randomly throughout the entire or a portion of hydrogel once polymerization is completed. Alternatively, a population of cells is seeded after the crosslinking reaction (polymerization reaction) is completed, or at least 70%, at least 80%, at least 90% of the crosslinking reaction (polymerization reaction) is completed. In such embodiments, the cells are injected into the hydrogel randomly or in a pattern. For example, in some embodiments, cells are seeded only on the surface or at near surface area of the hydrogel (e.g., within a depth of at most 10%, at most 20%, at most 30%, at most 40%, at most 50% of the entire depth or diameter of the hydrogel at a given point or location, etc.). In some embodiments, cells are seeded only or preferentially on one side of the hydrogel. In such embodiments, it is contemplated that at least 50%, at least 60%, at least 70%, at least 80%, at least 90%, or at least 95% of the cells are distributed in at most 60%, at most 50%, at most 40%, at most 30%, or at most 20% volume of the composition. In some embodiments the cells are seeded by a predetermined distance (e.g., every 2 mm, every 5 mm, every 10 mm, etc. or by increased or decreased distances between seeding locations throughout the hydrogel, etc.). In some embodiments, cells are seeded in a geometric pattern (e.g., square, triangle, round, rectangle, crosslines, pentagon, etc.) that is in vertical or horizontal plane of the hydrogel. In some cases, the crosslinking (or polymerization) conditions and/or cell seeding/mixing conditions are adjusted to reduce the toxicity to the cell or increase the cell viability to at least 30%, at least 40%, at least 50%, at least 60%, at least 70%, at least 80%, at least 85%, at least 90%, at least 95%, or at least 99% of cells initially mixed with the collagen-DAs mixture or seeded on the hydrogel would survive at least 1 day, at least 2 days, at least 3 days, at least 7 days, at least 14 days, at least 21 days, at least 30 days, at least 60 days after the polymerization reaction is completed or the hydrogel is implanted to the subject. In some embodiments, the pH of the collagen-DAS mixture is not optimal for cell survival (e.g., too acidic or too basic for the cells). In such embodiments, an optional step of neutralizing the collagen-DAS mixture (e.g., by addition of a buffering agent, NaOH, HEPES or NaHCO₃) is added before or during the addition of cells to the collagen-DAS mixture. Any suitable buffers to neutralize the collagen-DAS mixture or hydrogel are contemplated. In some embodiments, exemplary buffers comprise any zwitterionic buffer, or are selected from 2-(N-morpholino)ethanesulfonic acid (MES), 2-[(2-amino-2-oxoethyl)-(carboxymethyl)amino]acetic acid (ADA, also known as N-(2-acetamido)iminodiacetic acid), peperazine-N,N′-bis(2-ethanesulfonic acid) (PIPES, also known as 1,4-Piperazinediethanesulfonic acid), N-(2acetamido)-2-aminoethanesulfonic acid (ACES, also known as 2-(carbamoylmethylamino)ethanesulfonic acid), 2-hydroxy-3-morpholin-4-ylpropane-1-sulfonic acid (MOPSO), 3-(N-morpholino)propanesulfonic acid (MOPS, also known as 3-morpholinopropane-1-sulfonic acid), N,N-bis(2-hydroxyethyl)-2-aminoethanesulfonic acid (BES), 2-[[1,3,-dihydroxy-2-(hydroxymethyl)propan-2-yl]amino]ethanesulfonic acid (TES), (4-(2-hydroxyethyl)-1-piperazineethanesulfonic acid (HEPES), 3-(N,N-bis[2-hydroxyethyl]amino)-2-hydroxypropanesulfonic acid (DIPSO), Acetamidoglycine, 3-[[1,3-dihydroxy-2-(hydroxymethyl)propanyl-2-yl]amino]-2-hydroxypropane-1-sulfonic acid (TAPSO), piperazine-1,4-bis(2-hydroxypropanesulfonic acid) (POPSO), ((2-hydroxyethyl)-piperazine-N-2-hydroxypropanesulfonic acid (HEPPSO), 3-[4-(2-hydroxyethyl)piperazin-1-yl]propane-1-sulfonic acid (HEPPS, also known as EPPS), N-(2-hydroxy-1,1-bis(hydroxymethyl)ethyl)glycine (Tricine), 2-aminoacetamide (Glycinamide), 2(bis(2-hydroxyehtyl)amino) acetic acid (Bicine) or [tris(hydroxymethyl)methylamino]propanesulfonic acid (TAPS, also known as 3-{[1,3-dihydroxy-2-(hydroxymethyl)propan-2-yl]amino}propane-2-sulfonic acid). In some cases, a buffering agent comprises a zwitterion (e.g., a zwitterionic buffer such as MES, ADA, ACES, PIPES, BES, TES, HEPES, MOPS, MOPSO, DIPSO, TAPS, TAPSO, POPSO, HEPPSO, HEPPS, tricene, glycinamide, or bicine). In some cases, a buffer comprises saline or phosphate buffered saline. In some cases, a buffer comprises NaOH, HEPES, NaHCO₃ or a combination thereof. In some cases, a buffering agent is provided in solid (e.g. powder) or liquid forms. In some embodiments, NaOH is provided as a solution with a concentration of between about 0.5 normal (N) to about 4 N. In another example, HEPES is provided in a concentration of between about 100 millimolar (mM) to about 400 mM. In some cases, the NaHCO₃ in a concentration of between about 1.5 weight to volume percentage (w/v %) to about 3 (w/v %). In some cases, the neutralized collagen is less structured (e.g., lower stiffness, less physical stability) than the dialdehyde starch collagen DAS-COL. In an exemplary embodiment, FIG. 26A shows examples of dialdehyde starch crosslinked collagen (3 w/v %) with DAS (0.25 w/v %). In an exemplary embodiment, FIG. 26B shows neutralized collagen (3 w/v %). In some cases, a starch crosslinked collagen shows capability to form more stable three dimensional (3d) structures. In some cases, the neutralized collagen is less structured (e.g., more physical flexibility).

### (c) Methods of Bio-Printing

In some embodiments, such generated composition or collagen-DAS mixture is used a bioink for 3D printing of tissue substitutes. In some cases, the composition used as a bioink is a mixture of collagen and DAS (and optionally cells) that is not completely polymerized or crosslinked such that it maintains the fluidity or viscosity suitable for ejecting the composition through a nozzle of a bioprinting device. Thus, in some cases, the composition is the mixed collagen and DAS (and optionally cells) that are less than 80%, less than 70%, less than 60%, less than 50%, less than 40%, less than 30%, less than 20% crosslinked or polymerized at the time of ejection or when ready to be used as a bioink. Alternatively, in some cases, the composition is the mixed collagen and DAS (and optionally cells) that has viscosity of at most 1000 centipoise (CP), at most 900 CP, at most 800 CP, at most 700 CP, at most 600 CP, at most 500 CP, at most 400 CP, at most 300 CP, at most 200 CP, at most 100 CP, between 100 CP and 1000 CP, between 100 CP and 900 CP, between 100 CP and 800 CP, between 100 CP and 700 CP, between 100 CP and 600 CP, between 100 CP and 500 CP, between 200 CP and 500 CP, between 200 CP and 400 CP, etc. Thus, in some embodiments, cross-linking the polymers in the composition occurs after the composition is printed onto the substrate. Alternatively and/or additionally, cross-linking the polymers in the composition and printing occur simultaneously. Alternatively and/or additionally, in some embodiments, the composition comprises photoinitiator, and the composition is =exposed to the light (e.g., UV, visible light, etc.) after printing onto the substrate or during the extrusion from the bioprinting device.

In some embodiments, the bioink (mixed collagen and DAS (and optionally cells)) is deposited on the substrate using a bio-printing device that is configured to deposit a bio-ink composition onto the substrate. In some embodiments, the substrate comprises the tissue of a subject. For example, the substrate comprises a live, damaged tissue (e.g., damaged cartilage, teared cartilage, broken bone, etc.), or a space that the subject's tissue was located before injury or damage. In some cases, bioprinting of the composition is performed in vivo (e.g., during the surgery) directly onto the patient's tissue or ex vivo on the tissue that is temporarily removed from the subject or at least dislocated from the original location. In some cases, 3D printing of tissue substitute is performed in vitro. In such embodiments, the substrate comprises any sheet, frame or mold of any material (e.g., plastic, ceramic, metallic, carbon fiber, etc.). In some cases, it is contemplated that the in vitro printed tissue substitute is further customized to fit into the subject's damaged tissue or between broken tissues.

In some embodiments, the method comprises polymerization or degradation of the composition by exposure to electromagnetic radiation. In some embodiments the electromagnetic radiation comprises an electron beam, gamma-radiation, or UV radiation. In some embodiments, the method comprises degradation of the composition at least partially by exposure to light. In such embodiments, time, wavelength, and light intensity of light exposure vary depending on the size, location, and/or the chemical or mechanical properties of the composition. In some embodiments, degradation or polymerization are paused by shuttering the light. In some embodiments, the composition continues polymerizing or degrading once light exposure resumes. In some embodiments, the method further comprises removing composition components (e.g. non-cellular components, non-ECM components) after bioprinting by physical, chemical, or enzymatic means. In some embodiments, the composition components are removed by degradation of the composition components.

In some embodiments, the composition or hydrogel is printed into a three-dimensional tissue substitute using a bioprinter and/or a bioprinting system. Any suitable bioprinters or bioprinting systems are contemplated. For example, in some cases, a bioprinting system comprises a bio-printer, a substrate, a camera, and a computing device, which includes a processor that is operatively coupled to the bio-printer and to the camera, and a non-transitory computer readable storage medium with a computer program including instructions executable by the processor. The processor, upon receiving the instruction, is capable of i) convert an image of the substrate captured by the camera into a pattern that is recognizable by the bio-printer; and ii) provide instructions to the bio-printer instructing to deposit the bio-ink composition onto the substrate, in the shape of the pattern.

In some embodiments, the bioprinter comprises a printhead. In some embodiments, the printhead comprises a needle, an extended cylinder, a fluid line, a print nozzle, or a plurality of print nozzles. In some embodiments, the bioprinter comprises a second printhead.

In some embodiments, the bioprinting system or a bioprinter comprising a controller and a printhead. In some embodiments, the system comprises a controller. In some embodiments, the controller comprises a controller tip. In some embodiments, the controller tip comprises a printhead. In some embodiments, the printhead comprises a print nozzle. In some embodiments, the printhead comprises a plurality of print nozzles. In some embodiments, the printhead comprises a two-dimensional array of a plurality of print nozzles. In some embodiments, the system comprises a controller to control the printhead. In some embodiments, the controller is hand-held or mountable. In some embodiments, the controller is wireless. In some embodiments, the controller controls printhead parameters selected from: temperature; back-pressure; drops per nozzle; frequency of drop rate; number of nozzles in use; and firing energy, or a combination thereof. In some embodiments, the controller controls resolution, viscosity, cell concentration, physiological temperature and speed of printing. In some embodiments, the controller controls firing energy. In some embodiments, the firing energy comprises pulse energy, pulse width, length of gap between pulses, and voltage. In some embodiments, the printhead or controller further comprises a temperature control apparatus.

In some embodiments, the bio-printing systems disclosed herein comprise an extrusion bioprinting system. In some embodiments, the extrusion printing system comprises applying force, heat, or a combination thereof to eject the bio-ink. In some embodiments, the force is mechanical, pneumatic, or hydraulic force. In some embodiments, the extrusion printing system is a syringe. In some embodiments, the extrusion printing system ejects the bio-ink continuously. In some embodiments, the extrusion printing system ejects the bio-ink continuously when the force or heat is applied.

In some embodiments, the bio-printing systems disclosed herein comprise an ink-jet bioprinting system. Ink-jet printing is a printing technique that reproduces digital pattern information onto a substrate with ink drops. In some embodiments, the ink-jet printing system is a thermal ink-jet system. In some embodiments, the ink jet printing system is a piezoelectric ink jet system. In some embodiments, the ink jet printing system uses mechanical vibration. In some embodiments, the extrusion printing system is a diaphragm-based jetting implement.

In some embodiments, the inkjet printing system comprises a heating element in each print nozzle. In some embodiments, the heating element raises the local print nozzle temperature to about 100° C., about 150° C., about 200° C., about 250° C., about 260° C., about 270° C., about 280° C., about 285° C., about 290° C., about 295° C., about 298° C., about 300° C., about 302° C., about 305° C., about 310° C., about 315° C., about 320° C., about 325° C., about 350° C., about 375° C., or about 400° C. In some embodiments, the heating element raises the local nozzle temperature to about 300° C. In some embodiments, the heating element raises the temperature of the plurality of cells in the bio-ink about 1° C., about 2° C., about 3° C., about 4° C., about 5° C., about 6° C., about 7° C., about 8° C., about 9° C., about 10° C., about 11° C., about 12° C., about 13° C., about 14° C., or about 15° C. In some embodiments, the temperature of the plurality of cells in the bio-ink is raised for less than about 1 μsec, about 2 μsec, about 3 μsec, about 4 μsec, about 5 μsec, about 6 μsec, about 7 μsec, about 8 μsec, about 9 μsec or about 10 μsec. In some embodiments, the ink-jet printing system comprises one print nozzle. In other embodiments, the ink-jet printing system comprises a plurality of print nozzles.

Disclosed herein also includes methods of direct manufacturing of a tissue, tissue substitutes, or portion thereof within a tissue defect of a patient. The method comprises steps of i) positioning a printhead comprising a two-dimensional array of print nozzles within proximity of the tissue defect; and ii) ejecting a bio-ink comprising cells onto the tissue defect to produce a manufactured tissue in the tissue defect. Advantages of printing directly onto a tissue defect include, but are not limited to: i) eliminating the need for prior manufacturing, storage, or transportation; ii) providing the ability to customize the engineered tissue to perfectly fit defects of any shape or size; iii) the ability to vary the type or amount of tissue being generated during surgery; iv) the ability to combine artificial and natural scaffolds as well as living cells; and v) enabling direct integration of the newly printed tissue into the host tissue.

In some embodiments, the bioprinting system includes a computer system that comprises a processor, a memory device, an operating system, and a software module for monitoring or operating the printhead. In some embodiments, the computer system comprises a digital processing device and includes one or more hardware central processing units (CPU). In further embodiments, the computer system includes an operating system configured to perform executable instructions. In some embodiments, the operating system is software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux, and Palm® WebOS. In some embodiments, the computer system includes a storage and/or memory device. In some embodiments, the storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random-access memory (FRAM). In some embodiments, the non-volatile memory comprises phase-change random access memory (PRAM). In some embodiments, the device is a storage device including, by way of non-limiting examples, CD-ROMs, DVDs, flash memory devices, magnetic disk drives, magnetic tapes drives, optical disk drives, and cloud computing-based storage. In some embodiments, the storage and/or memory device is a combination of devices such as those disclosed herein.

In some embodiments, the computer systems described herein include user interfaces. In further embodiments, the user interfaces include graphic user interfaces (GUIs). In still further embodiments, the user interfaces are interactive and present a user with menus and options for interacting with the computer systems and bioprinters described herein. In further embodiments, the computer system includes a display screen to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of displays such as those disclosed herein. In still further embodiments, the device includes an input device to receive information from a user. In some embodiments, the input device is a keyboard. In further embodiments, the input device is a key pad. In a particular embodiment, the input device is a simplified key pad for use by a subject with communications limitations (e.g., due to age, infirmity, disability, etc.), wherein each key is associated with a color, a shape, and health/communication concept. In some embodiments, the input device is a pointing device including, by way of non-limiting examples, a mouse, trackball, track pad, joystick, game controller, or stylus. In some embodiments, the input device is the display screen, which is a touch screen or a multi-touch screen. In other embodiments, the input device is a microphone to capture voice or other sound input. In other embodiments, the input device is a video camera to capture motion or visual input. In still further embodiments, the input device is a combination of devices such as those disclosed herein. In some embodiments, the systems, and software modules disclosed herein are intranet-based. In some embodiments, the systems and software modules are Internet-based. In further embodiments, the computer systems and software modules are World Wide Web-based. In still further embodiments, the computer systems and software modules are cloud computing-based. In other embodiments, the computer systems and software modules are based on data storage devices including, by way of non-limiting examples, CD-ROMs, DVDs, flash memory devices, RAM (e.g., DRAM, SRAM, etc.), ROM (e.g., PROM, EPROM, EEPROM, etc.), magnetic tape drives, magnetic disk drives, optical disk drives, magneto-optical drives, solid-state drives, and combinations thereof.

### (d) Methods of Producing a Molded Scaffold

Further disclosed herein includes a method of producing a cell-laden, three-dimensional scaffold by depositing a mixture of the at least on population of cells and the collagen-DAS mixture or hydrogel onto a mold. In some embodiments, collagen-DAS mixture that is used as a substrate for molding is not completely polymerized or crosslinked such that it maintains the fluidity, elasticity, or viscosity suitable for shape changes in the mold. In some embodiments, the composition is mixed collagen and DAS (and optionally cells) that are less than 80%, less than 70%, less than 60%, less than 50%, less than 40%, less than 30%, less than 20% crosslinked or polymerized. In some embodiments, the collagen-DAS mixture or hydrogel used as a substrate for molding is 80-100%, 90-100% polymerized or cross-linked, and the polymerized or cross-linked hydrogel or mixture maintains mechanical stiffness less than 50 kpa, less than 40 kpa, less than 30 kpa, less than 20 kpa, less than 10 kpa, less than 5 kpa, less than 4 kpa, less than 3 kpa, less than 2 kpa, less than 1 kpa, between 0.5-50 kpa, between 1-40 kpa, between 1-30 kpa, between 1-20 kpa, between 2-10 kpa, such that the hydrogel or mixture changes its shape according to the shape of the mold when the hydrogel or mixture is placed and given a pressure of between 10 to 300 psi, between 10 to 200 psi, between 20 to 300 psi, between 20 to 200 psi, or between 50 to 200 psi.

In some cases, any suitable shape of mold is used to shape the collagen-DAS mixture or hydrogel. For example, shapes of molds includes a tissue, a portion of a tissue of any size, including, but not limited to a cartilage, a gum, a bone, at least a portion of a meniscus, at least a portion of an articular cartilage, at least a portion of a rib, at least a portion of an ear, at least a portion of a nose, at least a portion of a bronchial tube, at least a portion of an intervertebral disc, at least a portion of a maxillofacial cartilage, at least a portion of a temporomandibular joint (TMJ), at least a portion of a glenoid labrum, and at least a portion of an acetabular labrum, a cylinder, a cube, a three-dimensional rectangle, a sphere, a crescent, and a tetrahedron.

Also provided herein is a method of treating a bone or cartilage defect. In some embodiments, the method comprises transplanting a population of cells, as produced by the methods disclosed herein (e.g., a population of pluripotent or multipotent cells derived from chondrocytes and/or a population of chondrocytes or chondroprogenitors derived from a population of pluripotent or multipotent cells derived from chondrocytes), to the bone or cartilage defect. In some embodiments, new tissue is produced. In some cases, the method disclosed herein (e.g., a method of treating a bone or cartilage defect) comprises the use of a plurality of chondrocytes (e.g., in a mixture comprising dialdehyde starch, a collagen molecule or portion thereof, a heparin molecule or portion thereof, and/or a growth factor molecule or portion thereof). In some embodiments, the new tissue integrates with the tissue of the bone or cartilage defect. In some embodiments, the new tissue restores the surface of the cartilage or bone. In some embodiments, the new tissue comprises collagen type II. In some embodiments, the new tissue comprises superficial, intermediate, and deep zones characteristic of normal articular cartilage. In some embodiments, the superficial zone of the new tissue comprises lubricin.

Further disclosed herein, includes a method of regenerating cartilaginous tissue. In some embodiments, the method comprises a step of transplanting a population of cells, as produced by the methods disclosed herein (e.g., a population of pluripotent or multipotent cells derived from chondrocytes and/or a population of chondrocytes or chondroprogenitors derived from a population of pluripotent or multipotent cells derived from chondrocytes), to a bone or cartilage defect, wherein new cartilaginous tissue is produced. In some embodiments, the population of cells is transplanted into a bone or cartilage defect in a subject in need thereof. In many cases, a method disclosed herein (e.g., a method of regenerating cartilaginous tissue) comprises the use of a plurality of chondrocytes (e.g., in a mixture comprising dialdehyde starch, a collagen molecule or portion thereof, a heparin molecule or portion thereof, and/or a growth factor molecule or portion thereof). In some embodiments, new (e.g., regenerated) cartilaginous tissue integrates with the tissue of the bone or cartilage defect. In some embodiments, the new cartilaginous tissue restores the surface of the cartilage or bone. In some embodiments, the new cartilaginous tissue comprises collagen type II. In some embodiments, the new cartilaginous tissue comprises superficial, intermediate, and deep zones characteristic of normal articular cartilage. In some embodiments, the superficial zone of the new cartilaginous tissue comprises lubricin. In some embodiments, the new cartilaginous tissue does not comprise teratomas, neoplastic cells, evidence of deformation, abnormal architectural features, or other inappropriate cell types.

It is contemplated that bio-printed hydrogels are especially useful in filling cartilage defects with custom-shaped implants that, in some cases, are difficult to form with a mold or for cartilage defects that are too large for intraarticular injection.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, in some cases, “about” means within 1 or more than 1 standard deviation, per the practice in the given value. Where particular values are described in the application and claims, unless otherwise stated the term “about” should be assumed to mean an acceptable error range for the particular value.

The terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker).

“Treating” or “treatment” of a state, disorder or condition (e.g., cancer) includes: (1) preventing or delaying the appearance of clinical or sub-clinical symptoms of the disorder developing in a human that is afflicted with or pre-disposed to the disorder but does not yet experience or display clinical or subclinical symptoms of the disorder; and/or (2) inhibiting the disorder, including arresting, reducing or delaying the clinical manifestation of the disorder or at least one clinical or sub-clinical symptom thereof; and/or (3) relieving the disorder, e.g., causing regression of the disorder or at least one of its clinical or sub-clinical symptoms; and/or (4) causing a decrease in the severity of one or more symptoms of the disorder. The benefit to a subject to be treated is either statistically significant or at least perceptible to the patient or to the physician.

As used herein, a “bio-ink” refers to a composition suitable for bioprinting comprising a biopolymer and/or a plurality of cells. In some embodiments, bio-ink comprises cell solutions, cell aggregates, cell-comprising gels, proteins, multicellular bodies, or tissues.

As used herein, “chondrocytes” includes chondrocytes, articular chondrocytes, fibrochondrocytes, chondroblasts, chondrocyte precursors, chondrocyte progenitors, mesenchymal stem cells, osteoblasts, immature chondrocytes, cartilage cells, chondrogenic cells, osteogenic cells, osteoprogenitor cells, osteochondro progenitor cells, connective tissue fibroblasts, tendon fibroblasts, and cells that support the growth or differentiation of such cells.

As used herein, “polymerization” refers to both the process of forming a polymer chain and the process of forming networks of polymers. Polymerization includes cross-linking of polymers, including covalent and ionic cross-linking. In some embodiments, polymerization includes gelatinization, or gelling, of the compositions described herein. As used herein, the term “weight to volume” (abbreviated “w/v”) refers to a weight in grams of a component per 100 milliliters (mL) of a solution. For example, a composition comprising 10% w/v of dialdehyde starch would comprise 10 grams of dialdehyde starch per 100 milliliters of total solution.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Provision of Primary Human or Bovine Chondrocytes

This example shows a method for the provision of primary human chondrocytes for transplantation into a cartilage defect.

To obtain human primary chondrocytes, sterile scalpels were used to excise articular cartilage from femoral condyles and tibia plateaus of a healthy human subject under aseptic conditions. To obtain bovine chondrocytes, osteochondral plugs were removed from bovine femoral condyles under aseptic conditions. Harvested cartilage samples, for example osteochondral plugs, were minced and treated with 0.5 mg/mL trypsin at 37° C. for 15 min. The minced tissue was washed with 10% calf serum to remove trypsin solution and to deactivate any remaining trypsin. Next, the cartilage tissues were digested with 2 mg/mL type IV clostridial collagenase in DMEM with 5% fetal calf serum for 12 h to 16 h at 37° C. Released human articular chondrocytes were washed three times with DMEM supplemented with 1× penicillin-streptomycin-glutamine (PSG, 100 U/mL penicillin, 0.5 mg/mL streptomycin, 2 mM L-glutamine) The washing is generally performed at room temperature. Cell viability was determined using trypan blue exclusion and visual assessment of morphology. Isolated chondrocytes were seeded (plated) into T175 tissue culture flasks at 5 million cells per flask for expansion in monolayer and cultured in DMEM supplemented with 10% calf serum and 1×PSG. Cells were incubated at 37° C. with humidified air containing with 5% CO₂. The culture medium was changed every 4 days. Human chondrocytes were ready to be used in methods and compositions disclosed herein when 80% to 90% confluence was reached, which typically occurred from 1 to 2 weeks in primary cell culture. Primary human chondrocytes were typically used for treatment of a subject at passage one or passage two. In some embodiments, primary human chondrocytes or bovine chondrocytes provided in this manner were added to DAS-collagen matrices to generate compositions disclosed herein.

### Example 2: Chondrogenic Differentiation from Human Pluripotent Stem Cells

This example shows a method for providing human chondrocytes from human pluripotent stem cells.

H9 pluripotent stem cells (WiCell, Madison, Wis.) were maintained in culture in an undifferentiated state by passaging on irradiated human foreskin fibroblasts (line HS27, ATCC, Manassas, Va.) on gelatin coated plates. Gelatin is added to provide a coating to promote better cell proliferation. Cell differentiation was promoted using chemical compounds (e.g., a growth factor). To differentiate the pluripotent stem cells towards a mesodermal and then mesenchymal lineage, the colonies of the pluripotent stem cells were mechanically dissected into small pieces under microscopic guidance and then transferred to tissue culture-treated 6-well plates (Corning). The cells at this stage were considered passage 0 (P0). The cells were cultured in DMEM/F12 supplemented with non-essential amino acids and 10% fetal bovine serum (FBS, Invitrogen-Gibco, Grand Island, N.Y.). Cells were trypsinized before reaching confluency and were transferred to a new tissue culture flask.

Pluripotent stem cell-derived cells (2.5×10⁵ cells) were passaged at least five times using type II collagenase before being collected in 15-ml conical tubes and centrifuged at 150 g for 5 min after which they were transferred to serum-free chondrogenic media (Lonza, Basel Switzerland) in the presence of TGFβ3 (10 ng/ml; Peprotech, Rocky Hill, N.J.). The media was changed twice weekly. The passaging enzyme comprised 0.05% Trypsin-EDTA.

Chondroprogenitors were also differentiated from iPSC or human embryonic stem cells (hESC) (WAO9, Wicell) by culturing in DMEMIF12 containing 10% FBS. Chondrogenic differentiation of chondroprogenitors was performed by centrifuging 5×10⁵ cells to form a pellet and culturing the pellet in chondrogenic media (Lonza) containing TGFβ3 with BMP4 at about 10-100 ng/ml. The pellet culture was performed using hanging drops in a 15 ml conical tube.

At the end of 3 weeks of culture, some cell pellets were fixed with Z-Fix (Anatech, Battle Creek, Mich.), paraffin-embedded, sectioned, and assessed for their chondrogenic differentiation status using histochemical stains, immunocytochemical imaging, and qPCR.

For qPCR, total RNA was extracted from cell pellets with RNeasy kit (Invitrogen, Carlsbad, Calif.) and was reverse transcribed to cDNA with SuperScript (Invitrogen, Carlsbad, Calif) for confirmation of chondrocyte phenotype. After 2 weeks, type II collagen expression in these pellets was verified with RT-qPCR. Real-time RT-qPCR of collagen IIA1 and aggrecan was performed using Taqman® Gene expression assays as per manufacturer's instructions (Applied Biosystems, Foster City, Calif.).

For histochemical stains or immunocytochemical imaging, cell surface antigens on hESC-derived chondrocyte cells were analyzed by fluorescence-activated cell sorting (FACS). The cells were released from the tissue culture flask with Accutase, centrifuged, washed with phosphate buffered saline (PBS), and blocked in 2% FBS for 0.5 h at room temperature (RT). Cells (2×10⁵) were then incubated with each of the following using a BD Stemflow™ Human MSC Analysis Kit (BD Biosciences, San Jose, Calif), hMSC positive markers (CD73, CD90, CD105), and hMSC negative markers (CD11b, CD19, CD34, CD45, HLA-DR). After incubation, cells were washed and resuspended in PBS. Data were analyzed by collecting 20,000 events on a Cyan LX (Dako North America, Inc., Carpinteria, Calif.) instrument using WinMDI software. Nonspecific fluorescence was determined by incubation of similar cell aliquots with isotype-matched mouse monoclonal antibodies (PharMingen, San Diego, Calif) or with secondary antibody alone. Such differentiated or obtained human chondrocytes can be further added to DAS-collagen matrices to generate the disclosed composition.

### Example 3: Producing a Dialdehyde Starch-Collagen Hydrogel

This example shows a method for producing a dialdehyde starch (DAS)-collagen hydrogel for the treatment of cartilage defects.

DAS (Sigma) is dissolved in water at 70° C. and placed in a first syringe or container (e.g., as shown in FIGS. 3C-3D). Purified collagen dissolved in water is placed in a second syringe or container. DAS and collagen are mixed by connecting the first syringe to the second syringe with a syringe connector (male Luer-to-female Luer adapter, Cole-Palmer), for example as shown in FIGS. 3C-3E, and injecting the contents of one syringe into the other syringe, for example, by depressing the plunger of the first syringe and allowing the DAS to flow into the second syringe containing the collagen (e.g., as shown in the top panel of FIG. 3F). Improved mixing is achieved by subsequently depressing the plunger of the second syringe, causing the mixture of DAS and collagen to flow into the first syringe (e.g., as shown in the middle panel of FIG. 3F). Optionally, further mixing can be achieved by repeating the reversal of flow in the syringes described above one or more times (e.g., as shown in the bottom panel of FIG. 3F). In some cases, mixing is performed at a steady, moderate rate to avoid introduction of air bubbles into the composition and is continued until the composition is visually homogeneous. In some embodiments, the mixing is performed at room temperature. In some embodiments, the mixing is performed at a temperature between 18° C. to about 33° C.

Optional addition of heparin to DAS-Collagen hydrogel or mixture: Heparin in a 0.05 M MES buffer comprising 25 mM 1-ethyl-3-(3-dimethylaminopropyl)carboiimide and 10 mM N-hydroxysuccinimide is added to the DAS-collagen hydrogel at a concentration of 1 mg/ml and stirred for 4 hours. The hydrogel is washed three times with distilled water, and PDGF-BB is added to the hydrogel at a concentration of 100 ng/mg of hydrogel at a temperature of 4° C. for 4 hours. The hydrogel is then washed three times with PBS.

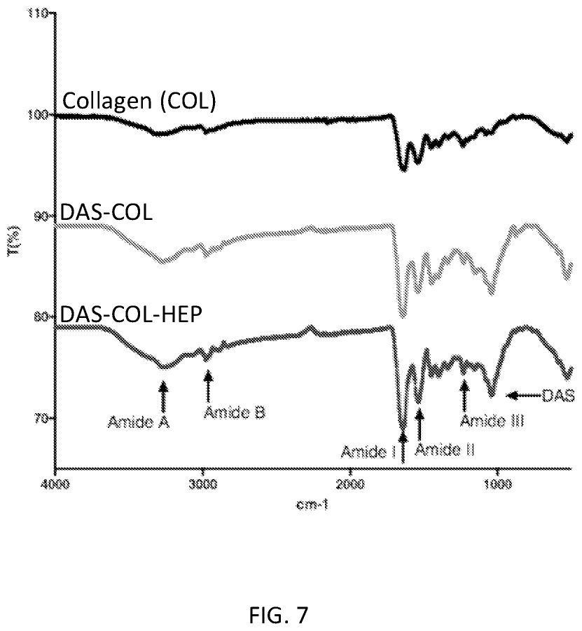

FIG. 7 shows Fourier transform infrared (FTIR) traces for collagen, DAS-collagen mixture (DAS-COL), and DAS-collagen-heparin mixture (DAS-COL-HEP) samples. The Amide A and B bands (approximately 3310 cm⁻¹ to 3030 cm⁻¹), Amide I band (approximately 1650 cm⁻¹), and DAS bands (approximately 1050 cm⁻¹) indicate successful bonding of DAS and collagen in the DAS-COL and DAS-COL-HEP compositions. Pronounced Amide I, Amide II (approximately 1550 cm⁻¹), and Amide III (approximately 1200 cm⁻¹) bands in the DAS-COL-HEP trace indicate successful bonding of heparin to the DAS-COL backbone. Heparin is masking the DAS-COL hydrogel.

### Example 4: Various Conditions for Polymerization of a Dialdehyde Starch-Collagen Hydrogel or Molded Scaffold

This example describes the effects of adjusting reagent concentration and time of polymerization on the mechanical properties of dialdehyde starch-collagen (DAS-COL) compositions described herein.

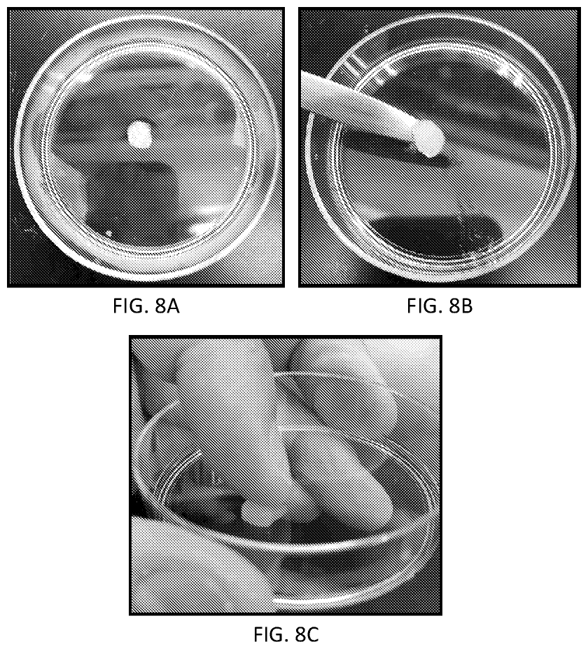

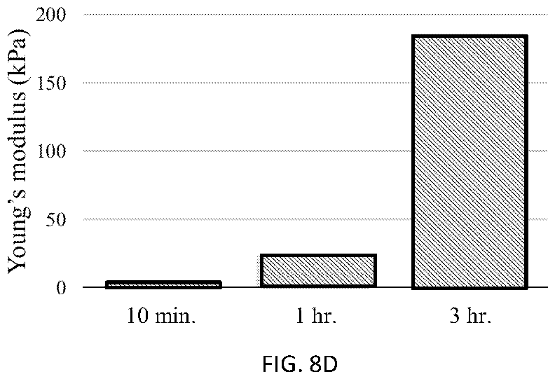

Hydrogels formed using the method described in Example 3 were subjected to qualitative visual and compressive evaluation of mechanical stiffness at 10 minutes, 1 hour, and 3 hours after mixing of DAS and collagen reagents. Hydrogels exhibited high viscosity and an ability to flow at 10 minutes after mixing (FIG. 8A). At 1 hour after mixing, hydrogels exhibited visual and compressive characteristics of a viscoelastic solid (FIG. 8B). At 3 hours after mixing, hydrogels exhibited mechanical characteristics of a resilient solid (FIG. 8C). The timing of pH neutralization of mixed DAS-COL hydrogels is adjusted based on the desired viscosity and stiffness of the hydrogel used in applications described herein. FIG. 8D shows measured Young's modulus values for DAS-COL hydrogels at 10 minutes (2.48 kPa), 1 hour (24.28 kPa), and 3 hours (185.3 kPa) after mixing DAS and collagen. Young's modulus values were higher at 1 hour than 10 minutes and higher at 3 hours than 1 hour.

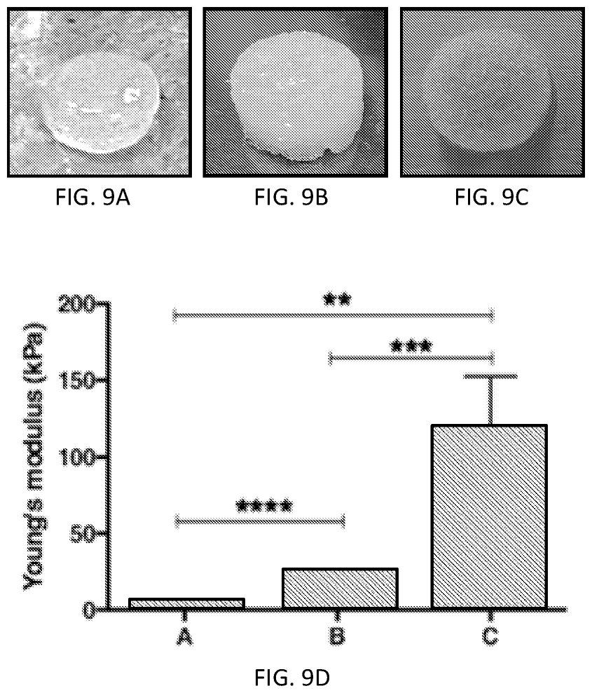

pH neutralization was achieved during testing by including various concentrations of NaOH in a buffering reagent used during hydrogel synthesis. Hydrogels formed in the presence of 0.25 N NaOH were able to be molded into cylindrical (e.g., disc-shaped) plugs and exhibited viscoelastic solid mechanical characteristics 3 hours after DAS and collagen were mixed (FIG. 9A). Hydrogels formed in the presence of 0.5 N NaOH were able to be molded into cylindrical plugs and showed viscoelastic solid mechanical characteristics 3 hours after DAS and collagen were mixed (FIG. 9B). Hydrogels formed in the presence of 1.0 N NaOH were able to be molded into cylindrical plugs and showed resilient solid characteristics (FIG. 9C). FIG. 9D shows measured Young's modulus values for DAS-COL hydrogels formed using (A) 0.25 N NaOH buffer (2.48 kPa), (B) 0.5 N NaOH buffer (24.25 kPa), or (C) 1.0 N NaOH buffer (118.57 kPa) at 3 hours after mixing. Hydrogel stiffness was significantly higher for hydrogels formed using 0.5 N NaOH (B) than those formed using 0.25 N NaOH (A). Hydrogel stiffness was significantly higher for hydrogels formed using 1.0 N NaOH (C) than those formed using 0.5 N NaOH (B). A one-way ANOVA test was used to evaluate statistical differences (** p<0.005, *** p<0.001, **** p<0.0001). An Instron Universal Testing Machine, 3342 Single Column Model was used with a 500N load cells at 1 mm/min speed to perform the test described herein.

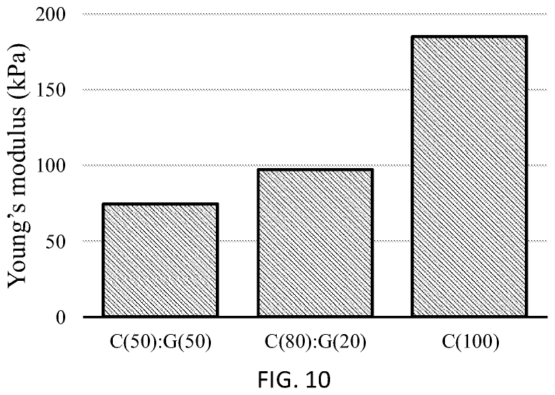

FIG. 10 shows that the measured Young's modulus of hydrogels comprising collagen to gelatin at a 4:1 ratio (C(80):G(20)) is higher than the measured Young's modulus of hydrogels comprising collagen to gelatin at a 1:1 ratio (C(50):G(50)). Mechanical testing shows that the Young's modulus of hydrogels comprising collagen to gelatin at a 4:1 ratio (C(80):G(20)) is less than that of compositions comprising 100% collagen (C(100)).

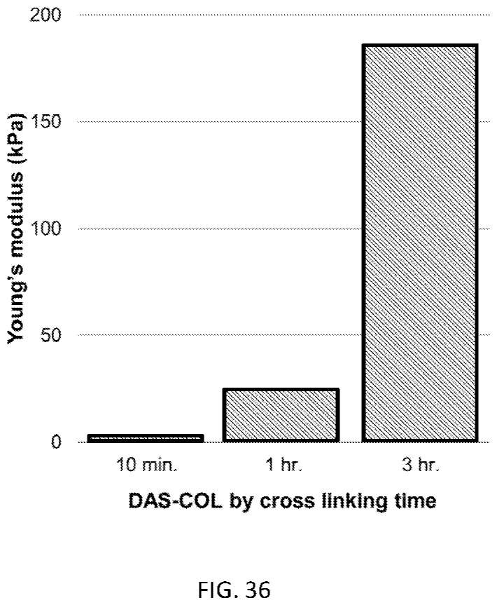

A similar mechanical testing was performed to analyze the effect of crosslinking time on the Young's modulus of a DAS-COL hydrogel cylinder. The DAS-COL hydrogel cylinder was formed by mixing 4% w/v collagen and 10% w/v DAS were mixed in a volume ratio of 9:1. FIG. 36 shows that increasing the crosslinking time from 10 minutes to 3 hours can significantly increase the stiffness of a cylindrical block of a DAS-COL gel.

FIG. 11 shows that a dialdehyde starch-collagen-heparin (DAS-COL-HEP) hydrogel (8% w/v collagen) prepared as in Example 2 has sufficient mechanical stiffness and consistency to be successfully sutured. This data indicates that DAS-COL-HEP hydrogels can be sutured in situ in a surgical setting.

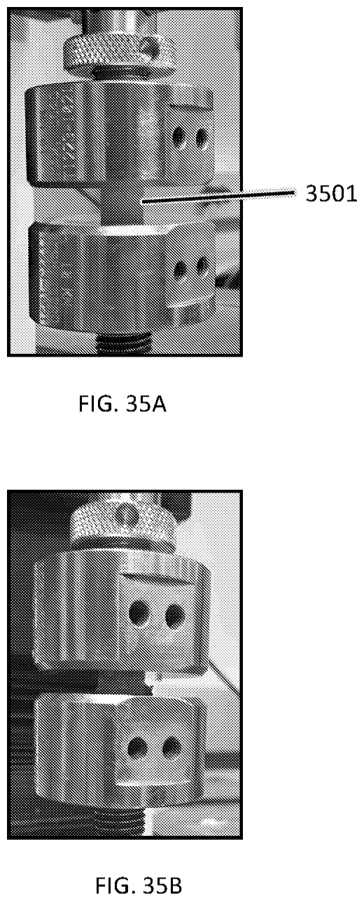

FIG. 35A-35B show an example of a compression test performed on a DAS-COL gel. Cylindrical gels were made using about 4% w/v collagen and about 10 w/v DAS in a volume ratio of 9:1. A compression test apparatus comprising a load cell 3501 was used to test the compression of the cylindrical DAS-COL gels. A cylindrical DAS-COL gel 3501 was placed in the compression apparatus (FIG. 35A). The compression apparatus provided an axial force to the sample to compress the sample. A height difference before and after compression was measured to calculate a percentage height change. FIG. 35B shows DAS-COL gel cylindrical block was compressed up to 50% of its initial height (e.g., a before breakage test). In some cases, there irreversible loss of height was observed. In some embodiments, a DAS-COL based gel is compressed to more than 50% of its initial (or original) dimension (e.g., height, width, thickness or length).

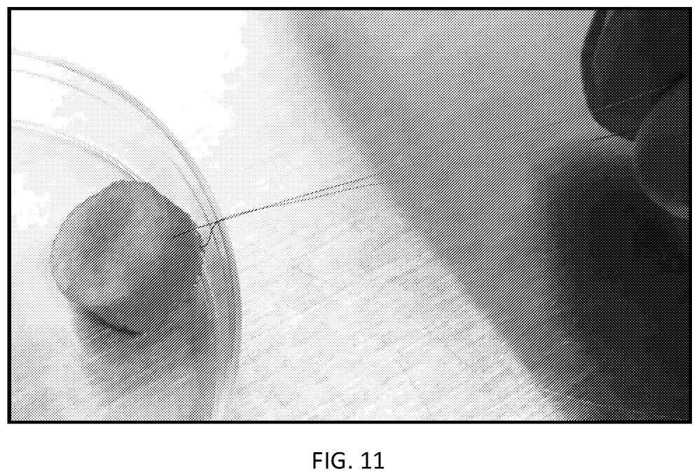

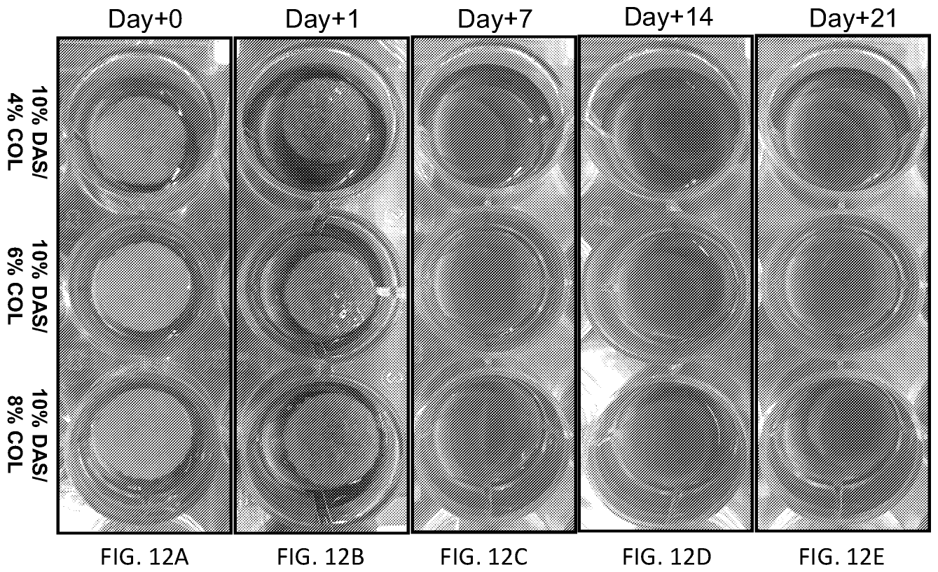

Degradation of molded DAS-COL hydrogel plugs in culture medium (DMEM) at 37° C. in the presence of 0.75 N NaOH was observed for various ratios of DAS content to collagen content (10% w/v DAS to 4% collagen; 10% DAS to 6% collagen, and 10% DAS to 8% collagen). Degradation of plugs was apparent in all hydrogels one day after placing into culture (Day +1, FIG. 12B) compared to the Day 0 (FIG. 12A). While hydrogel plugs exhibited a slightly more rounded shape at Day +1 than Day 0, the diameter of the hydrogel plugs was approximately the same at Day +0 and Day +1. In 4% gel, the diameter reduced about 1³0.93% at D +1, and 18.03% at D +7 comparing to D +0. At Days+14 and 21 the diameter did not change significantly comparing to D +7. In 6% and 8% gels, the diameter reduced about 4.10% at D +1. The diameter remained did not change considerably at other time points.

By Day +7 (FIG. 12C), all hydrogel plugs were noticeably smaller in diameter than those hydrogel plugs at either Day +1 or Day 0. Hydrogels also exhibited a slightly ellipsoid shape in all cases at Day +7. Hydrogel plugs comprising 4% collagen were noticeably smaller and more ellipsoid than those comprising 6% or 8% collagen. The dimensions and shapes of hydrogel plugs at Day +14 (FIG. 12D) were comparable to those observed at Day +7. By Day +21 (FIG. 12E), hydrogels maintained a rounded (e.g., ellipsoid or cylindrical) shape. Sizes of hydrogel plugs were comparable between 4% collagen, 6% collagen, and 8% collagen samples at Day +21. These results show that DAS-COL hydrogels of various collagen compositions are suitable for implantation in vivo, as they maintain overall structure and size for 21 days in culture while allowing for progressive degradation and integration or release of hydrogel components (e.g., cellular integration with host tissue and/or release of growth factors associated with the hydrogel).

### Example 5: Cell Seeding on a Dialdehyde Starch-Collagen Hydrogel or Molded Scaffold

This example shows successful incorporation of chondrocytes into DAS-collagen (DAS-COL) hydrogels.

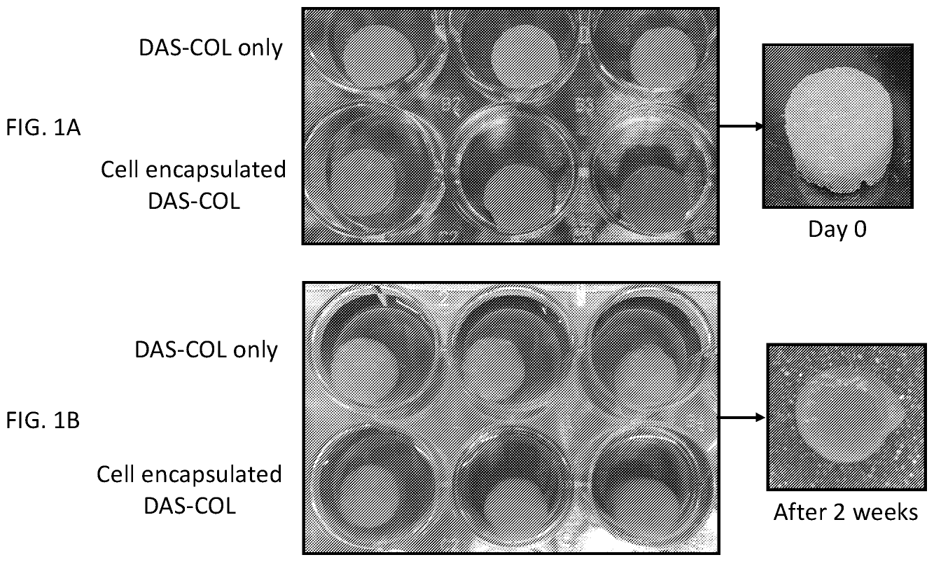

Bovine chondrocytes provided by the method of Example 1 or Example 2 are seeded in DAS-COL hydrogels prepared as in Example 4 and formed into a cylindrical plug using a mold. About 7 million bovine chondrocytes were applied to half of the DAS-COL hydrogel plugs. Cells were seeded on both sides (e.g., top and bottom) of the plugs. 50 □□□ of cell media was added to the top, and the bottom of each plug using a pipette. FIG. 1A shows initial morphology of DAS-COL hydrogel plugs seeded with bovine chondrocytes or human chondrocytes (lower wells) and/or unseeded with chondrocytes (upper wells) at Day 0. Cells were allowed to infiltrate the hydrogels for 3 days (FIG. 3A). On day 3, hydrogels were removed from culture, stained with calcein am and ethidium homodimer-1 (Invitrogen) and imaged in a longitudinal cross-section using confocal microscopy to assess viability of cells within the hydrogel plugs (FIG. 3B). Most cells showed green staining (cells showing positive staining in FIG. 3B grayscale image not identified with an arrow), with only a few cells showing red ethidium homodimer-1 staining (cells identified with an arrow in FIG. 3B). These results show that chondrocytes are able to infiltrate and evenly distribute through DAS-COL hydrogels in vitro and that the vast majority of cells in cell-seeded hydrogels are viable in both interior and superficial regions of the hydrogel plugs.

Cylindrical plugs of chondrocyte-seeded DAS-COL cultured in vitro for 2 weeks using DMEM (FIG. 1B, lower wells) showed more degradation of DAS-COL matrix than unseeded DAS-collagen cultured hydrogel plugs under the same conditions for 2 weeks (FIG. 1i, upper wells).

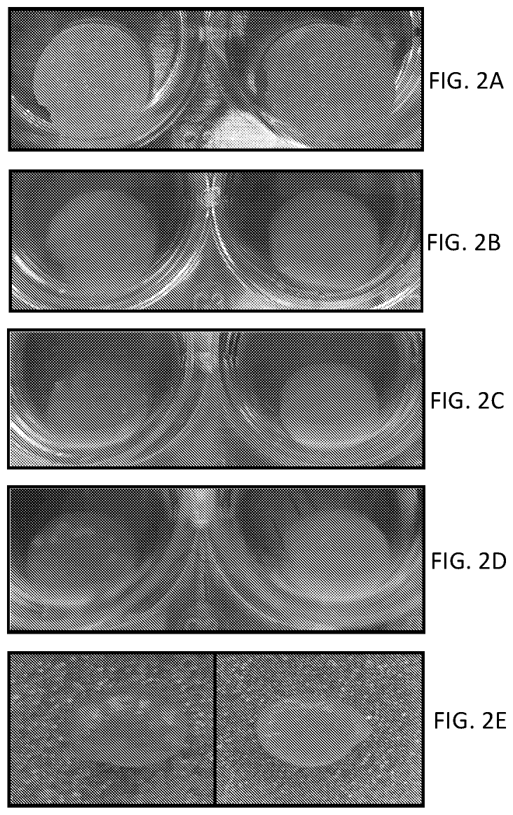

When degradation experiments were performed with molded, bovine chondrocyte-seeded dialdehyde starch-collagen-heparin (DAS-COL-HEP) hydrogel plugs seeded with 5 million chondrocytes, using DAS-COL-HEP hydrogels cultured in insulin, transferrin, sodium selenite (ITS) either supplemented with TGF-□3 (FIGS. 2A-2E, right side) or without TGF-□3 (FIGS. 2A-2E, left side), hydrogels were observed to degrade at similar rates and in similar morphologies regardless of TGF-□3 treatment at Day 1 (FIG. 2B), Day 7 (FIG. 2C), and Day 14 (FIG. 2D), compared to Day 0 (FIG. 2A). When hydrogels were removed from culture at Day 14 and examined, similar morphologies and sizes were observed in control-treated (ITS alone, FIG. 2E left side) and TGF-□3-treated hydrogels (ITS+TGF-□3, FIG. 2E right side).

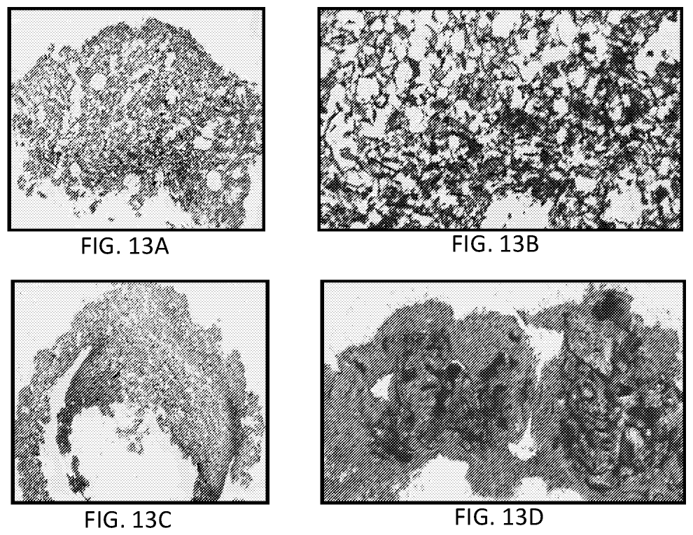

Day 14 hydrogels were sectioned and stained with safranin O to assess glycosaminoglycan (GAG) deposition in the hydrogel (FIGS. 13A-13D). Chondrocyte-seeded DAS-COL hydrogels cultured in ITS+TGF-b3 medium (FIG. 13C, 10× magnification objective; FIG. 13D, 40× magnification objective) showed increased cellular proliferation and GAG deposition than chondrocyte-seeded DAS-COL hydrogels culture in ITS medium (FIG. 13A, 10× magnification objective; FIG. 13B, 40× magnification objective). The ITS media comprises 1×ITS (e.g., from a 100× stock), 0.1 mM ascorbic acid 2-phosphate, 1.25 mg/ml human serum albumin, 10−7M dexamethasone, 1×PSG (penicillin/streptomycin/glutamine) with DMEM media, and 10 ng/ml TGF-□3.

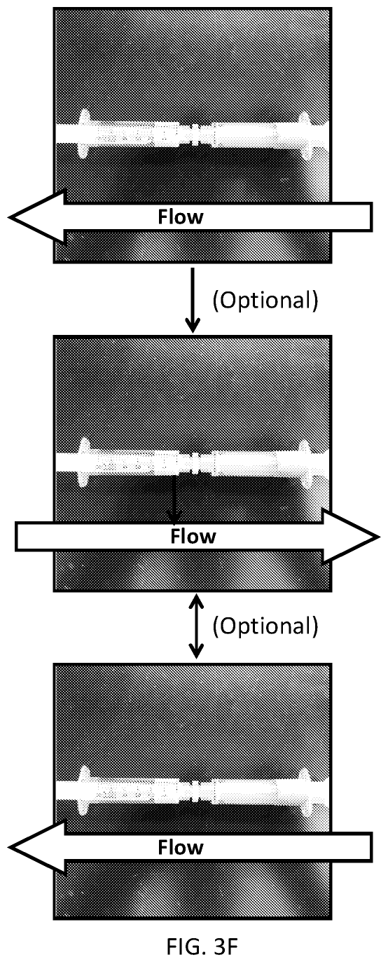

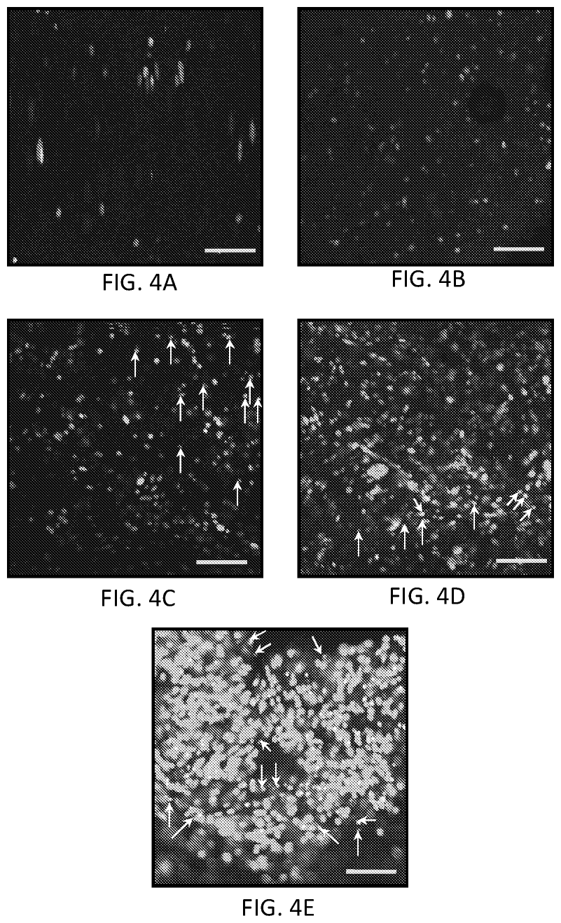

Viability of cells seeded onto dialdehyde starch-collagen-heparin (DAS-COL-HEP) hydrogels was evaluated 2 days after cell seeding using Calcein-AM-EthD-1 staining. In this staining method, live cells are distinguished by the presence of ubiquitous intracellular esterase activity, determined by the enzymatic conversion of the virtually nonfluorescent cell-permeant calcein AM to the intensely fluorescent calcein. The polyanionic dye calcein can be retained within live cells, producing an intense uniform green fluorescence in live cells (ex/em ˜495 nm/˜515 nm). EthD-1 can enter cells with damaged membranes and undergoes a 40-fold enhancement of fluorescence upon binding to nucleic acids, thereby producing a bright red fluorescence in dead cells (ex/em ˜495 nm/˜635 nm). EthD-1 can be excluded by the intact plasma membrane of live cells. In FIGS. 4A-4E dead cells or non-viable cells are marked by arrows. In these figures, arrows indicate cells stained as non-viable. Cells were seeded at densities of 1 million cells/mL (FIG. 4A), 2 million cells/mL (FIG. 4B), 4 million cells/mL (FIG. 4C), 8 million cells/mL (FIG. 4D), and 10 million cells/mL (FIG. 4E). Positive staining in FIGS. 4A-4E indicates viable cells except for cells indicated by arrows, which stained positive for ethidium homodimer-1 in the viability testing assay. Results show that the vast majority of cells present in DAS-COL-HEP hydrogels are viable. The scale bars in FIGS. 4A-4E indicate 50 □m and arrows depicted in the same figures indicate cells stained as non-viable.

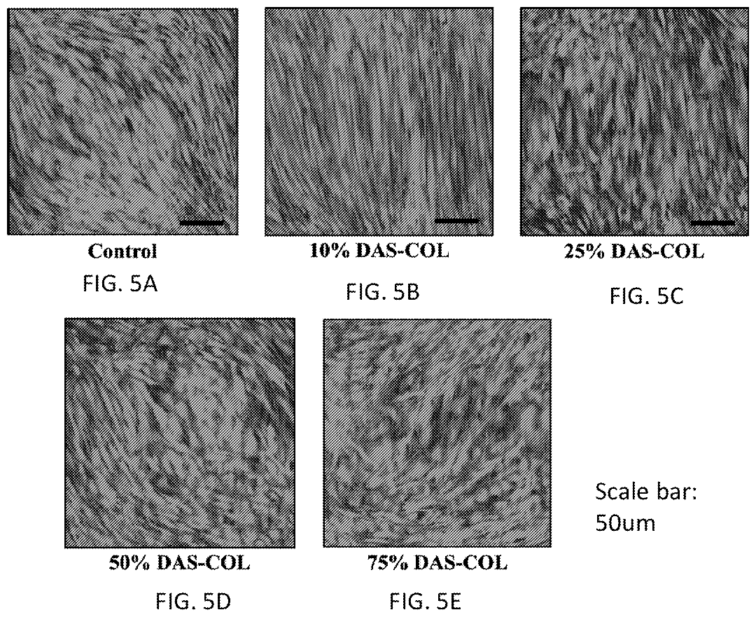

When DAS-COL hydrogels of varying DAS-COL concentrations were seeded with human meniscal cells and stained with a viability dye (calcein AM and ethidium homodimer-1, Invitrogen) after 3 days in culture, human meniscal cells were observed to be viable in all cases (FIGS. 5A-5E). Cells were seeded by adding the cells to the surface of the hydrogel. The scale bars in FIGS. 5A-5E represent 50 □m. In DAS-COL hydrogels comprising 100% collagen (FIG. 5A), 10% DAS with 90% collagen (FIG. 5B), 25% DAS with 75% collagen (FIG. 5C), 50% DAS with 50% collagen (FIG. 5D), and 75% DAS with 25% collagen (FIG. 5E), human meniscal cells were found to be uniformly viable (positive staining indicates viable cells). No decrease in cell viability was observed with increasing DAS concentration in DAS-COL mixture.

### Example 6: Cell Embedding within a Dialdehyde Starch-Collagen Hydrogel or Molded Scaffold

This example shows a method for forming DAS-COL hydrogels (e.g., DAS-COL bio-inks) comprising living cells.

Prior to mixing dialdehyde starch and collagen, 10 million human or bovine cells, such as chondrocytes, were added to the container comprising dialdehyde starch (DAS) described in Example 3. The mixture of DAS and cells was then mixed with the collagen, as described in Example 3. In order to embed the cells in the hydrogel, the hydrogel mixture was neutralized with a neutralizing agent and cells were mixed within the hydrogel substantially immediately after neutralization. Mixed DAS-COL hydrogels with embedded cells were then formed into a desired shape using a mold or applied directly to a target site.

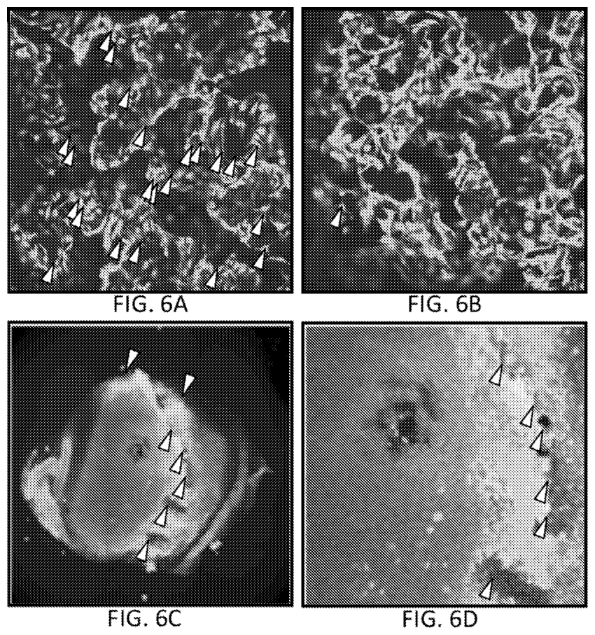

Viability testing was performed on cultured DAS-COL hydrogels formed with embedded cells using calcein am and ethidium homodimer-1 viability staining (Invitrogen). Good viability was observed at 1 day (FIG. 6A), 9 days (FIG. 6B), and 21 days (FIG. 6C) of culture after hydrogel formation (positive staining indicates viable cells except for cells identified with arrowheads, which stained positively for ethidium homodimer-1). FIG. 6D shows a high-power view of the day 21 hydrogel shown in FIG. 6C.

In a separate preparation, human or bovine cells, such as chondrocytes, were added to the container comprising collagen described in Example 3. The mixture of collagen and cells was then mixed with the collagen, as described in Example 3. Mixed DAS-COL hydrogels with embedded cells were formed into a desired shape using a mold or applied directly to a target site.

### Example 7: Addition of Supplements in a Dialdehyde Starch-Collagen Hydrogel or Molded Scaffold

This example shows the characterization of a growth factor to a dialdehyde-starch-heparin (DAS-COL-HEP) hydrogel.

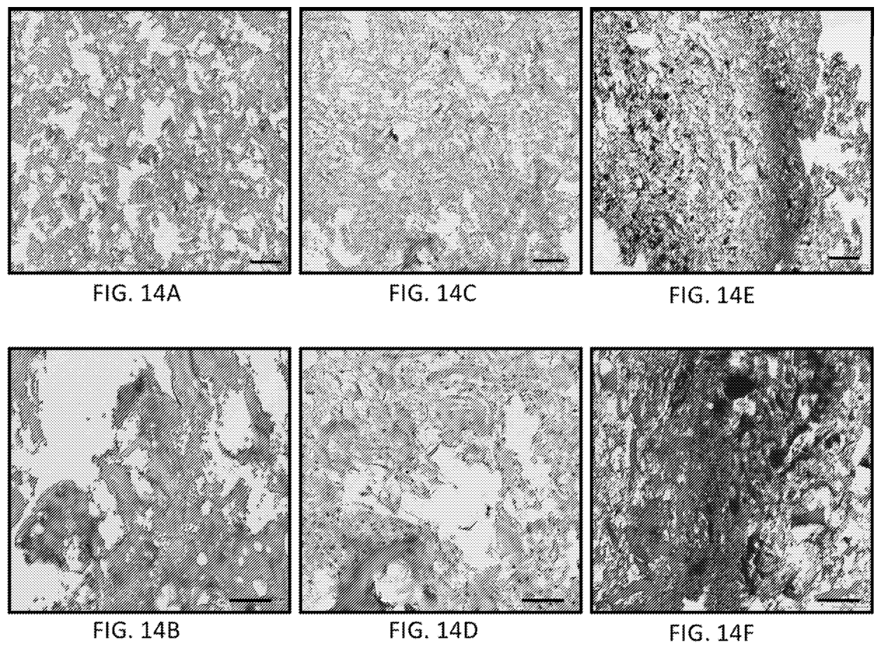

Bovine chondrocytes were mixed with DAS-COL-HEP hydrogel and insulin-like growth factor-1 (DAS-COL-IGF1), transforming growth factor beta 3 (DAS-COL-TGF□3), or no growth factor (DAS-COL-HEP) and formed into cylindrical plugs in a mold before being placed in culture with DMEM supplemented with insulin-transferrin-sodium selenite (ITS) for three weeks. The collagen was first dissolved using acetic acid, DAS was then added followed by adding the heparin (HEP). A concentration of a growth factor (e.g., 10 nanogram per milliliter (ng/ml) of TGF D 3) was added to the mixture. The mixed DAS-COL gel is neutralized by 10× phosphate buffered saline (PBS), and a neutralizing buffer. DAS-COL-HEP, DAS-COL-IGF1, and DAS-COL-TGF □3 plugs were characterized using safranin-O staining (FIGS. 14A, 14D, and 14G, respectively). After 3 weeks in culture, safranin-O-stained sections of DAS-COL-IGF1, and DAS-COL-TGF □3 hydrogels showed more positive staining than DAS-COL-HEP hydrogels, indicating that the presence of growth factors in DAS-COL hydrogels could increase production of glycosaminoglycans by chondrocytes embedded into the hydrogels (FIGS. 14D and 14G compared to FIG. 14A). These results are shown in higher magnification images in FIGS. 14E (DAS-COL-IGF1), 14H (DAS-COL-TGF □3), and 14B (DAS-COL-HEP). More safranin O was observed in DAS-COL-TGF□3 hydrogels than DAS-COL-IGF1 hydrogels after three weeks in culture (compare FIG. 14G to FIG. 14D and FIG. 14H to FIG. 14E).

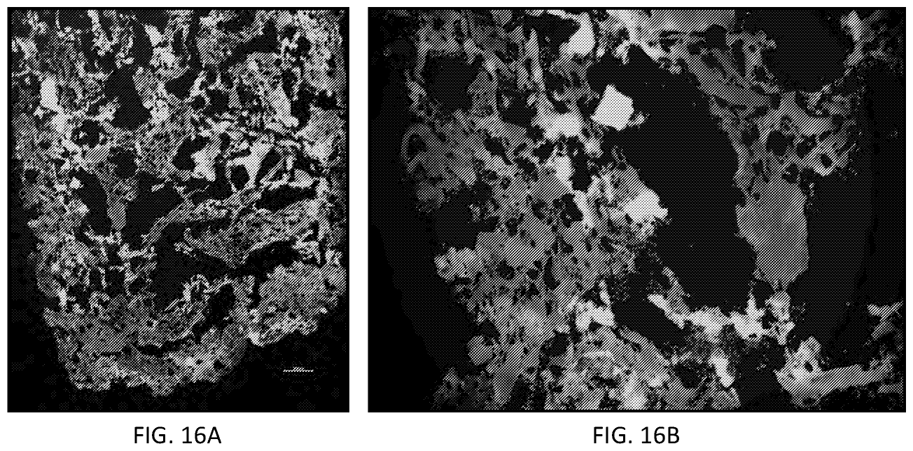

When 40 kilodalton (kDa) dextran was conjugated to IGF-1 prior to incorporation into DAS-COL-HEP hydrogels comprising bovine chondrocytes, fluorescent imaging showed that IGF-1 was evenly and well-distributed throughout the hydrogel at 21 days of culture (see FIGS. 16A and 16B, punctate staining is Hoechst nuclear stain, indicating cell nuclei).

### Example 8: Cell Growth in a Dialdehyde Starch-Collagen Hydrogel or Molded Scaffold

This example shows growth and viability of bovine chondrocytes in DAS-COL hydrogels prepared with or without growth factors.

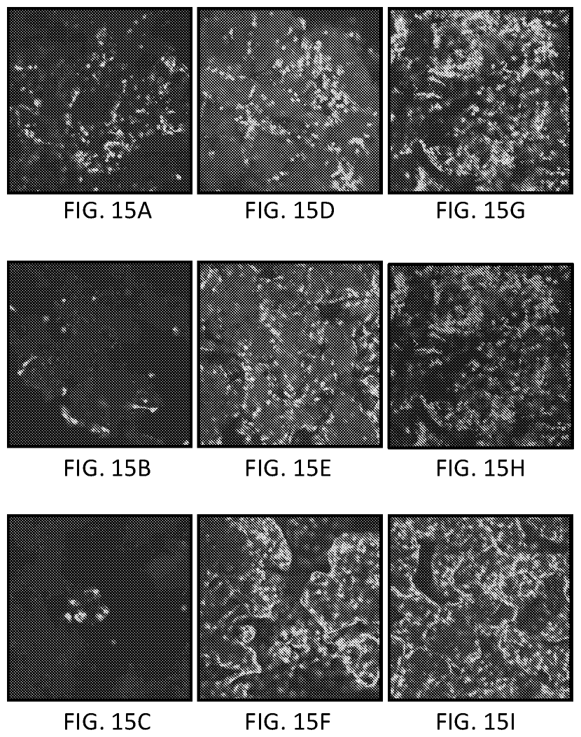

DAS-COL-HEP hydrogels were mixed with bovine chondrocytes and cultured for three weeks as described in example 7. DAS-COL-HEP hydrogels showed viable chondrocytes at Day +1 (FIG. 15A), Day +9 (FIG. 15B), and Day +21 (FIG. 15C) in culture and a decrease in the number of cells staining positive for ethidium homodimer-1, indicating a decrease in the number of dead cells over time.

DAS-COL-HEP hydrogels were mixed with bovine chondrocytes and IGF-1 (DAS-COL-IGF1 hydrogels) and cultured for three weeks. DAS-COL-IGF1 hydrogels showed strong viable chondrocyte staining at Day +1 (FIG. 15D), Day +9 (FIG. 15E), and Day +21 (FIG. 15F) in culture and a low number of cells staining positive for ethidium homodimer-1 at each time point.

DAS-COL-HEP hydrogels were mixed with bovine chondrocytes and TGF-1 (DAS-COL-TGF□1 hydrogels) and cultured for three weeks. DAS-COL-TGF1 hydrogels showed strong viable chondrocyte staining at Day +1 (FIG. 15G), Day +9 (FIG. 15H), and Day +21 (FIG. 15I) in culture and a low number of cells staining positive for ethidium homodimer-1 at each time point.

FIGS. 16A-16B show fluorescent images of DAS-COL-IGF1 hydrogels seeded with bovine chondrocytes and cultured for 21 days, as described herein. FIG. 16B shows a magnified portion of the hydrogel section shown in FIG. 16A. FIGS. 16A and 16B show fluorescence-labeled 40 kDa dextran conjugated to IGF-1 of the hydrogel; punctate staining in FIGS. 16A and 16B indicates cellular nuclei (Hoechst staining).

These results show that the inclusion of growth factors in DAS-COL hydrogels that comprise chondrocytes improves cell viability, especially at early time points.

### Example 9: Cell Migration Assay Using a Dialdehyde Starch-Collagen Hydrogel or Molded Scaffold

This example shows that growth factors associated with DAS-COL hydrogels exhibit functional chemotactic activity.

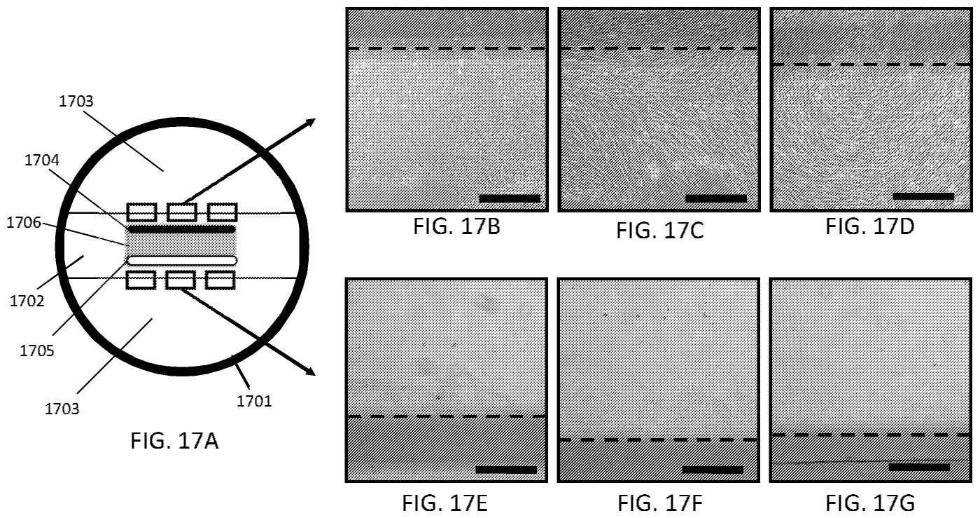

A 30 mm wide migration channel 1702 were cleared from a 10 cm culture dish 1701 grown to 90% confluence with bovine chondrocytes 1703 using a cell scraper, as shown in FIG. 17A. Separate compositions of DAS-COL-HEP mixed with 200 ng/mL PDGF-BB (DAS-COL-PDGF-BB; FIG. 17A, element 1704) and DAS-COL-HEP hydrogel without PDGF-BB (DAS-COL-HEP; FIG. 17A, element 1705) were printed along opposite long edges of a 50 mm×22 mm glass coverslip 1706. The coverslip was then placed in the migration channel 1702 of the culture dish 1701, and the culture dish 1701 was cultured in DMEM with ITS at 37° C. for 3 days. Images of the migration channel along each side of the coverslip 1706 were imaged at Day 3 (e.g., as indicated by black boxes in FIG. 17A) and quantified for migration of bovine chondrocytes into the migration channel region 1702 of the culture dish surface. FIGS. 17B-17D show migration of bovine chondrocytes into the migration channel 1702 (dotted lines indicate original edge of cultured bovine chondrocytes following scraping) on the side of the channel nearest to the DAS-COL-PDGF-BB hydrogel. Scale bars represent 500 i m. FIGS. 17E-17G show migration of bovine chondrocytes into the migration channel 1702 (dotted lines indicate the original edge of cultured bovine chondrocytes following scraping) on the side of the channel nearest to the DAS-COL-HEP hydrogel. Results show that DAS-COL hydrogels comprising growth factors exert a chemotactic effect on cells in a region to which the DAS-COL hydrogel is deployed.

### Example 10: Verification of Heparin Content on Dialdehyde Starch-Collagen Hydrogels

This example shows a method of quantifying heparin content of a dialdehyde starch-collagen (DAS-COL) hydrogel.

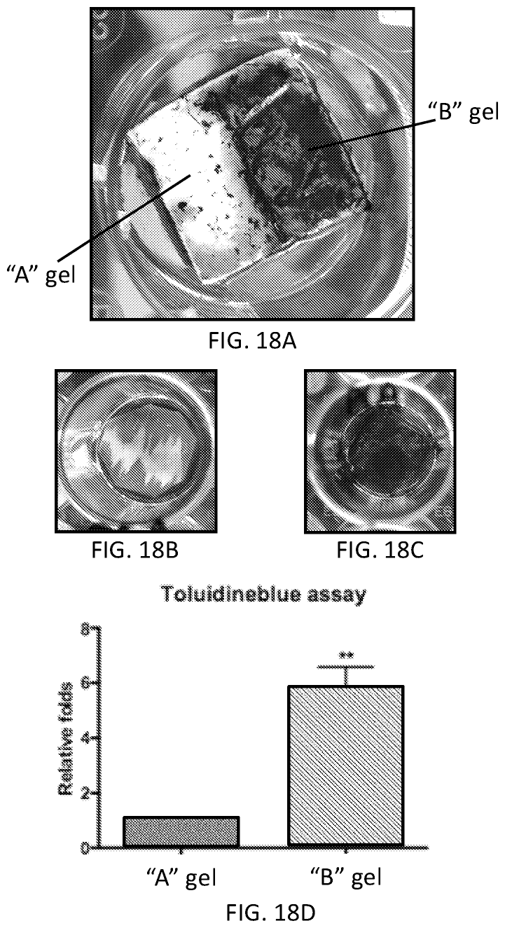

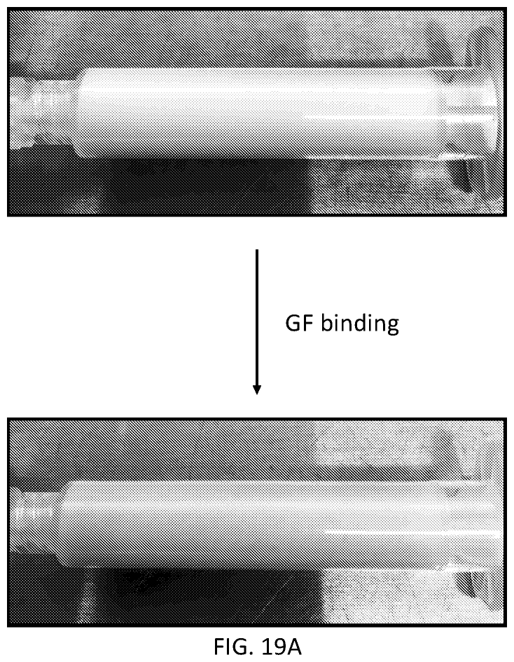

DAS-COL hydrogels were prepared by mixing the DAS-COL hydrogel with heparin sodium salt (0.1% w/v) either before a neutralization step of DAS-COL hydrogel formation (“A” gel as shown in FIG. 18A and FIG. 18B) or after a neutralization step of DAS-COL hydrogel formation (“B” gel, or “DAS-COL-HEP,” as shown in FIG. 18A and FIG. 18C), as described herein. “A” gels and “B” gels were printed on individual 1 cm×1 cm hydrophobic polylactic acid (PLA) sheets (e.g., as shown in FIGS. 18B and 18C, respectively) or on portions of the same PLA sheet (e.g., as shown in FIG. 18A). After 4 hours, PLA sheets comprising “A” gel and/or “B” gel were stained with 0.4 mg/mL toluidine blue to determine an amount of heparin on each hydrogel. PLA sheets were washed three times with distilled water before being extracted using a mixture of 0.1 M NaOH (20% v/v) and absolute ethanol (80% v/v). Hydrogel-bound heparin was quantified using a microplate reader. As shown in FIG. 18D, heparin was successfully associated with DAS-COL hydrogels when heparin was added either before or after a neutralization step; however, significantly more heparin was associated with DAS-COL hydrogels when heparin was mixed with the DAS-COL hydrogel after a neutralization step (e.g., “B” gels).

### Example 11: Molding a Dialdehyde Starch-Collagen Hydrogel

This example shows methods for molding a dialdehyde starch-collagen (DAS-COL) hydrogel into a desired shape.

DAS-COL hydrogels were mixed with growth factors in a syringe in the presence of a buffer to neutralize the acidic pH of the hydrogel and to allow the growth factors to associate with the molecules of the DAS-COL hydrogel (FIG. 19A). DAS-COL hydrogels comprising growth factors were deposited into a cylindrical tube (e.g., a syringe with the tip removed), compressed between two pistons (e.g., syringe plungers), and allowed to incubate at room temperature for 3 hours (FIG. 19B). After 3 hours, molded cylindrical DAS-COL hydrogel plugs were placed in culture dish wells and a buffer was added during the steps of blocking, cell seeding, blocking, or delivery to a target tissue. FIG. 19C. Optionally, molded DAS-COL hydrogels can be lyophilized into DAS-COL sponges for storage and/or transportation (FIG. 19D).

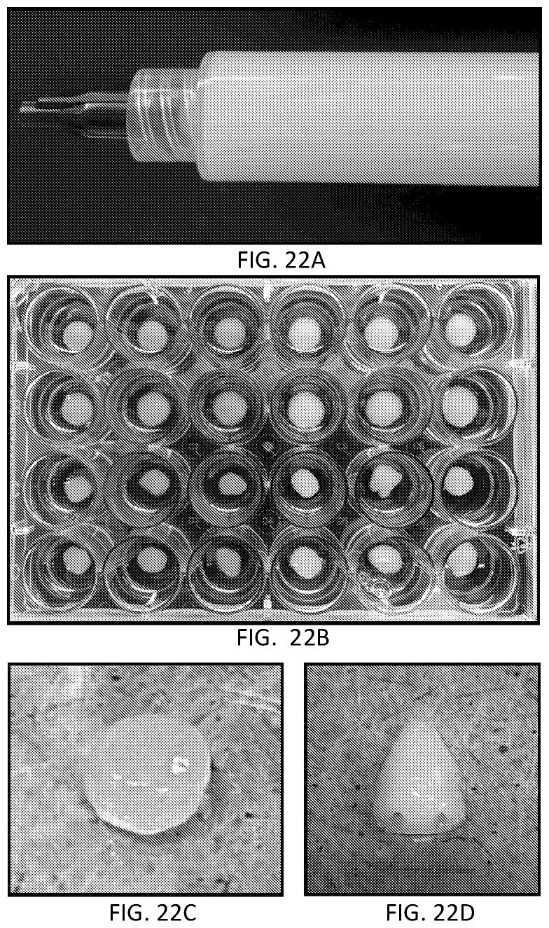

FIG. 22A shows a DAS-COL hydrogel embedded with cells and loaded into a syringe from which it is ready to be extruded into a patterned hydrogel or into a target location of a subject's body (such as a knee joint). FIG. 22B shows twenty-four DAS-COL hydrogels embedded with cells that were extruded into various shapes, including ellipsoid and disc shapes, in culture wells. FIG. 22C shows a cylindrical disc shaped DAS-COL hydrogel after extrusion. FIG. 22D is an image of a DAS-COL hydrogel that was molded into the shape of a nose.

### Example 12: Layered Dialdehyde Starch-Collagen Hydrogel

This example shows the generation of layered dialdehyde starch-collagen (DAS-COL) hydrogels.

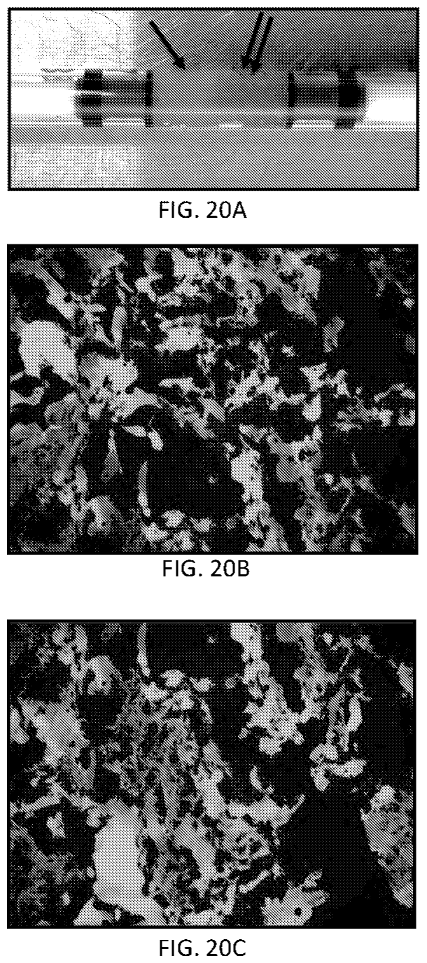

A layered DAS-COL hydrogel comprising a first layer of DAS-COL matrix bound to IGF-1 and embedded with 10 million bovine chondrocytes per mL hydrogel and a second layer of DAS-COL matrix bound to TGF-□3 and embedded with 10 million bovine chondrocytes per mL hydrogel was formed in a cylindrical mold (FIG. 20A). To create the layers of the hydrogel, each of two aliquots of DAS-COL-heparin matrix (DAS-COL-HEP) was mixed separately with IGF-1 or TGF-□3, and the two aliquots were loaded into a cylindrical mold adjacent to and in physical contact with one another. Plungers were used to compress the two layers of hydrogel together, and the layered cylindrical plug of hydrogel was placed into a tissue culture plate for 3 weeks with DMEM supplemented with insulin, transferrin, and sodium selenite (ITS). FIG. 20B shows fluorescent imaging of a first layer of the two-layer DAS-COL hydrogel shown in FIG. 20A comprising bovine chondrocytes and IGF-1 conjugated to 40 kDa dextran. FIG. 20C shows fluorescent imaging of a second layer of the two-layer DAS-COL hydrogel shown in FIG. 20A comprising bovine chondrocytes and TGF-□3 conjugated to 40 kDa dextran. Together, these data show that a DAS-COL hydrogel can be formed to include a plurality of distinct layers, one of which comprising a first growth factor (e.g., IGF-1) and the other of which comprising a second growth factor (e.g., TGF-□3).

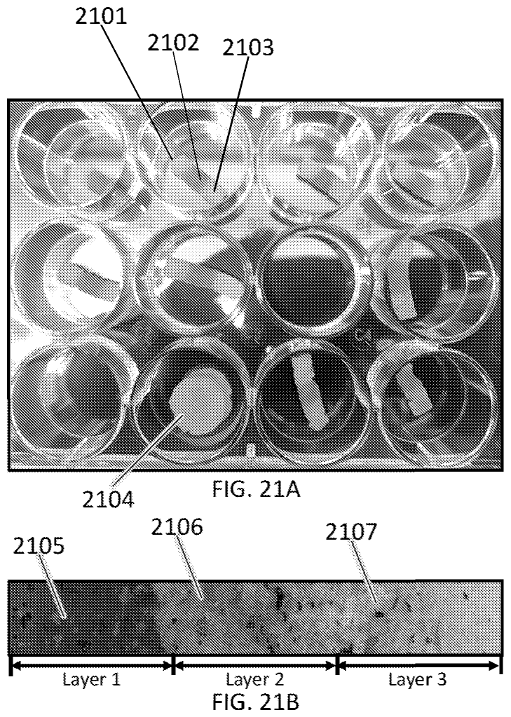

FIG. 21A shows various DAS-COL hydrogels with heparin comprising three distinct layers, each comprising a first layer comprising a hydrogel 2101, a second layer comprising a first growth factor (e.g., IGF-1) 2102, a third layer comprising second growth factor (e.g., TGF-□3) 2103. The first layer sometimes comprises a third growth factor, which is optionally the same as the first growth factor or the second growth factor or different than either (e.g., PDGF-BB). A 4% collagen solution was mixed with a 5% DAS solution and HEP (10 ug/ml). The growth factor IGF-1 (10 ng/ml) couple to dextran expressing yellow 2102 and a growth factor TGF-□3 (10 ng/ml) mixed with dextran expressing red 2103 were mixed with DAS-COL-HEP gel. Three layers of the hydrogel (or gel), made as described herein, were loaded into the Icc syringe mold. A first layer gel was loaded into the syringe mold first followed by the second layer gel, followed by the third layer gel. 2104 shows an example of a three layer hydrogel with a TGF-□3 layer on top and an IGF-1 layer in the middle, as described hereinbefore.

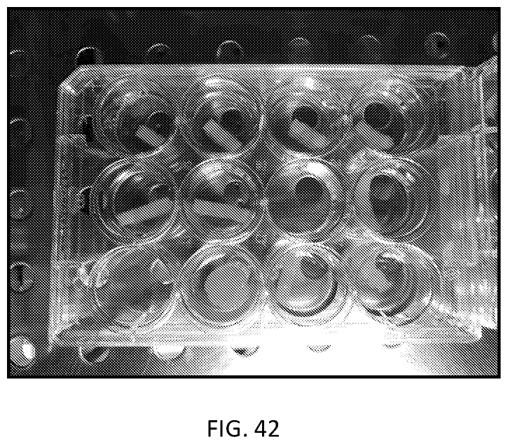

FIG. 21B shows a composited microscope image of a longitudinal section of a DAS-COL hydrogel with heparin shown in FIG. 21A comprising three distinct layers; 2105 contains no Dextran, 2106 contains yellow Dextran-conjugate, 2107 contains green Dextran-conjugate. FIG. 42 shows the same DAS-COL hydrogels with three different layers, as mentioned herein, degeneration test in medium culture. The gels maintained integrity in the medium culture for 21 days.

### Example 13: Preparation of Dialdehyde Starch-Collagen Hydrogels in Various Delivery Formats

This example shows that DAS-COL hydrogels can be prepared as bio-inks, unmolded viscous solids, and molded hydrogel plugs for use in various biological settings.

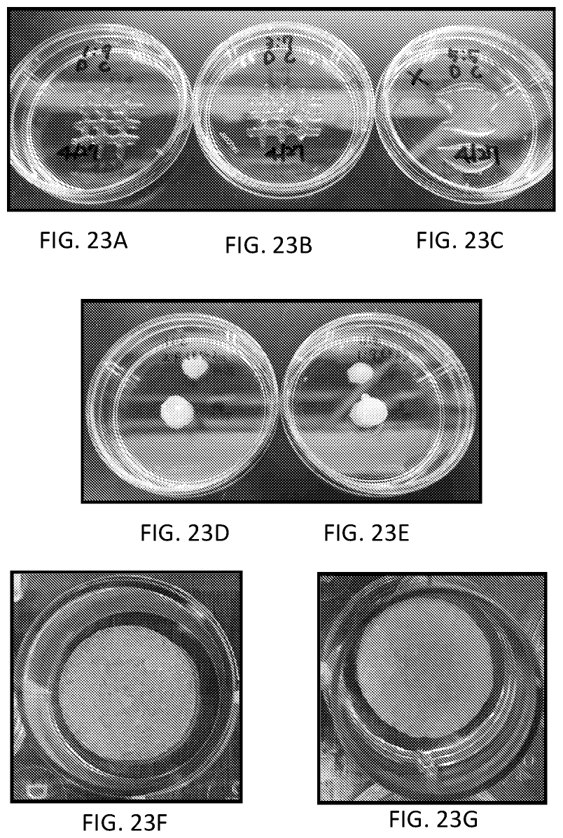

FIG. 23A-23C show various compositions of DAS-COL hydrogels formulated as bio-inks. A 1% solution of DAS and a 4% solution of collagen (COL) were prepared. 23A shows a DAS-COL hydrogel comprising a 1:9 ratio of 1% DAS solution to 4% collagen solution that has been printed in a pattern on the dish surface. 23B shows a DAS-COL hydrogel a 3:7 ratio of 1% DAS solution to 4% collagen that has been printed in a pattern on the dish surface. 23C shows a DAS-COL hydrogel a 1:1 ratio of 1% DAS solution to 4% collagen that has been deposited onto the dish surface. The ability of the hydrogel to retain the shape in which it was printed increases with increasing ratio of collagen to DAS. The ease of injection/deposition increased with decreasing ratio of collagen to DAS.

FIG. 23D-23E show DAS-COL hydrogels that have been printed onto the surface of culture dishes. A 10% solution of DAS and an 8% solution of collagen (COL) were prepared. FIG. 23D shows a hydrogel comprising a ratio of DAS to COL of 2:8, which is 2 parts 10% DAS solution mixed with 8 parts 8% collagen solution. FIG. 23E shows a hydrogel comprising a 1:9 ratio of a 10% solution of DAS and an 8% solution of collagen.

FIGS. 23F-23G show DAS-COL hydrogels that have been molded into cylindrical discs and placed into culture dishes. The hydrogel in FIG. 23F comprises a 1:9 mixture of DAS to collagen made using 10% DAS and 4% collagen. The hydrogel in FIG. 23G comprises a 1:9 mixture of DAS to collagen made using 10% DAS and 8% collagen. The molded hydrogels of FIGS. 23F and 23G are subsequently delivered to a target region and, optionally, sutured in place, in accordance with methods described herein. Optionally, a molded hydrogel, such as those shown in FIGS. 23F and 23G, is seeded with cells in accordance with the methods described herein prior to delivery to a target tissue.

### Example 14: Automated Production of a Dialdehyde Starch-Collagen Molded Scaffold

This example shows a method for automated production of a DAS-collagen hydrogel for the treatment of cartilage defects.

DAS (Sigma) is dissolved in water at 70° C. using a stir bar and then added to a first reservoir of a hydrogel preparation system. Purified collagen from a second reservoir of the hydrogel preparation system is added to the solution at a ratio of 4 parts collagen to 1 part DAS via a computer-controlled fluidic system. The mixture is conveyed to a mold via the fluidic system and allowed to cross-link for 1 hour at 4° C. while being stirred.

Heparin in a 0.05 M MES buffer comprising 25 mM 1-ethyl-3-(3-dimethylaminopropyl) carboiimide and 10 mM N-hydroxysuccinimide is added to the DAS-collagen hydrogel from a third reservoir at a concentration of 1 mg/ml and stirred for 4 hours. The hydrogel is washed three times with distilled water, and PDGF-BB is added to the hydrogel at a concentration of 100 ng/mg of hydrogel at a temperature of 4° C. for 4 hours. The hydrogel is then washed three times with PBS.

Chondrocytes provided by the method of Example 1 or Example 2 are seeded in the prepared DAS-collagen hydrogel by flowing the chondrocytes into a reservoir containing the hydrogel from a fourth reservoir via a fluidic system. Chondrocyte-seeded DAS-collagen are cultured in vitro for 2 weeks using DMEM, at which time they are ready for implantation into a cartilage defect.

The use of an automated system to prepare a DAS-collagen implant allows for faster, more precise, and more repeatable hydrogels to be made for implantation in a cartilage defect of a subject.

### Example 15: Transplantation of DAS-COL-Chondrocytes Hydrogel in Bovine Knee

This example shows successful implantation of DAS-COL hydrogels into bovine knee defects.

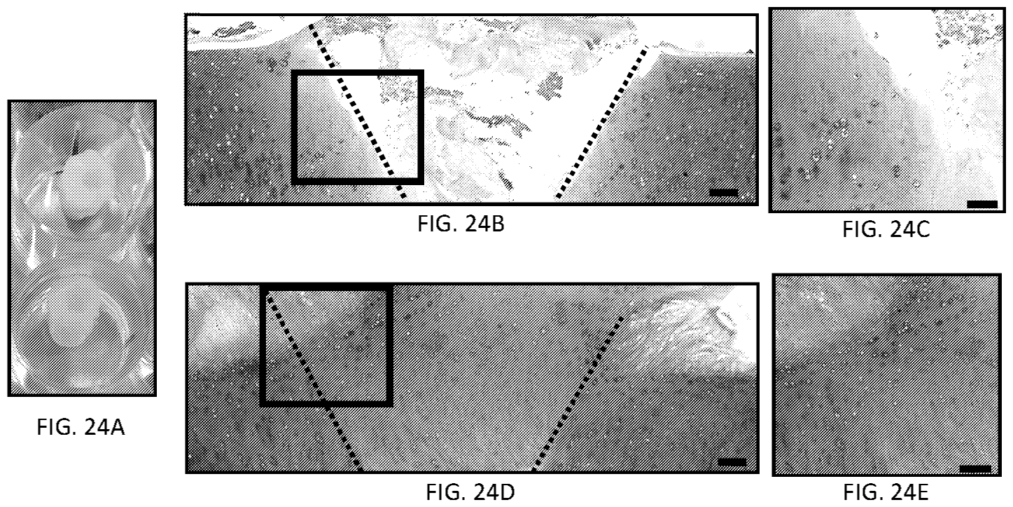

FIG. 24A shows a DAS-COL hydrogel formed into a cylindrical plug and seeded with 10 million cells (top well) and a DAS-COL-IGF1-TGF □3 hydrogel formed into a cylindrical plug and seeded with 10 million cells (bottom well). FIG. 24B shows a sectioned bovine knee articular cartilage defect three weeks after implantation of the control DAS-COL hydrogel in the top well of FIG. 24A (dotted lines indicate the original border of the defect made in the articular cartilage). FIG. 24C shows a higher magnification image of the portion of FIG. 24B indicated by the black box. The scale bar represents 200 □m. The staining was Safranin O. FIG. 24D shows a sectioned bovine knee articular cartilage defect three weeks after implantation of the DAS-COL-IGF1-TGF G 3 hydrogel in the bottom well of FIG. 24A (dotted lines indicate the original border of the defect made in the articular cartilage). At three weeks after implantation, cellular tissue in the defect is continuous and integrated with the cellular tissue outside of the defect. FIG. 24E shows a higher magnification image of the portion of FIG. 24D indicated by the black box. Incorporation of growth factors IGF-1 and TGF-□3 increased the cellular growth in the bovine cartilage defect at 3 weeks compared to defects treated with DAS-COL hydrogels that did not comprise growth factors.

### Example 16: Repairing Cartilage Defects in Explants from Adult Human Arthritic Joints by Transplantation

This example shows a method for filling an articular defect using tissue from a donor location in a subject's joint.

Osteochondral specimens were surgically resected from the joints of adult arthritic human patients undergoing total knee replacement. Six-mm diameter cylindrical plugs are cored out of donor locations of the subjects' joints with an Arthrex Single Use OATS System (Naples, Fla.). A surgical curette was used to make partial-thickness defects approximately 2 mm in size in the articular surface of a recipient subject's joint. The defects were filled with pluripotent stem cell-derived chondrogenic precursors which had been aggregated under the following mechanical pressures: 5×10⁵ cells centrifuged in 15-ml conical tubes at 150 g for 5 min in DMEM/F12 supplemented with 10% FBS and incubated overnight in the presence or absence of TGFβ. After 4 weeks, explants were fixed, paraffin-embedded, sectioned, and stained with Safranin O.

This method of treating a cartilage defect is most useful in subjects having acceptable donor locations for cartilage transplantation or subjects with a compatible cartilage donor subject.

### Example 17: Repairing Cartilage Defects in Adult Human Arthritic Joints by Intraarticular Injection

Intraarticular injection of DAS-collagen matrix seeded with chondrocytes allows for minimally invasive treatment of a defect site. The following technique is a representative example of the use of DAS-Collagen compositions described herein to repair cartilage defects in human articular joints.

A chondrocyte-seeded DAS-collagen hydrogel is provided according to Example 5. The chondrocyte-seeded DAS-collagen hydrogel is chilled to 4° C. and loaded into a chilled syringe. Injections of 0.3 ml of the chondrocyte-seeded DAS-collagen matrix per defect site are made in target cartilage tissue of a subject during a surgery. Follow-up evaluations of the injected cartilage tissue are performed periodically, and additional injections of DAS-collagen matrix seeded with chondrocytes are given as needed to maintain the repaired site.

### Example 18: Bioprinting of DAS-Collagen Hydrogels

This example shows a method of treating cartilage defects using bio-printed DAS-collagen hydrogel seeded with chondrocytes.

A chondrocyte-seeded DAS-collagen hydrogel is provided in accordance with the methods of Example 5, with DAS from a first reservoir being mixed with collagen from a second reservoir and chondrocytes of a third reservoir of a bioprinting platform via a fluidic system controlled by a computer with a graphical interface. A bioprinting platform with a two-dimensional printhead of 300 dots or nozzles per square inch is set at a distance of 1 to 2 mm from the substrate. Patterns with the shape and size of the cartilage defect of mold are designed using a compatible software program (e.g., Adobe® Photoshop®) and printed layer by layer to fabricate a three-dimensional construct shaped to fill a cartilage defect of a patient. Printed cell-hydrogel constructs are cultured with DMEM supplemented with 1× insulin-transferrin-selenium, 0.1 mM ascorbic acid 2-phosphate, 1.25 mg/ml human serum albumin, 10⁻⁷ M dexamethasone, 1×PSG, and 10 ng/mL TGF-β1 to maintain chondrogenic phenotype of the chondrocytes and then surgically implanted into a defect site in the cartilage of a patient.

### Example 19: Mixing Procedure of a Collagen Composition

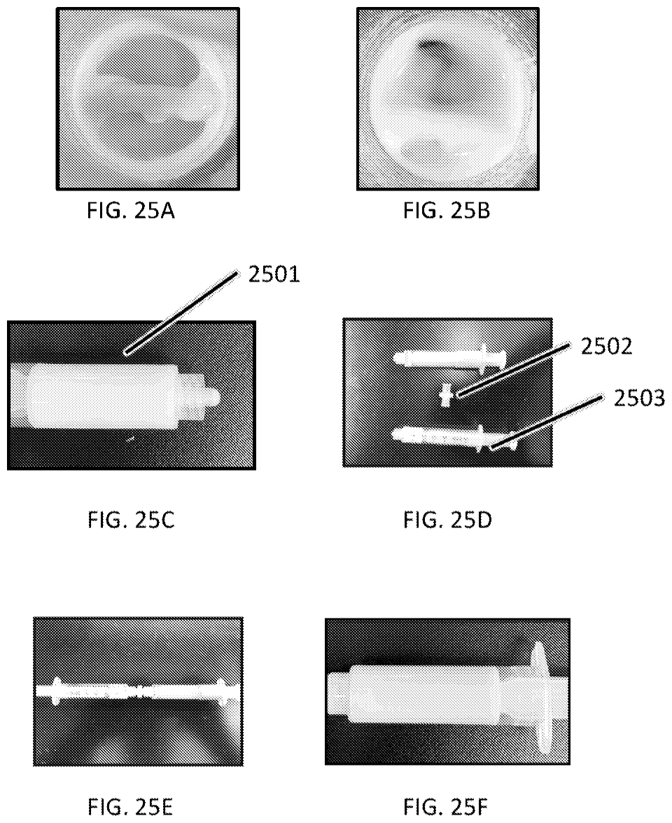

Two different collagen compositions comprising DAS-COL or DAS-COL-STARCH were formed using this mixing procedure. First collagen (2˜4 w/v %) was dissolved with acetic acid (0.1M) (FIG. 25A). Then, collagen was mixed with first dialdehyde starch (0.1˜10 w/v %) followed by 10×DPBS (Dulbecco's phosphate-buffered saline); at this step, starch (15 w/v % at a DAS-COL-STARCH ratio of 1:8:1) was mixed to form a gel (FIG. 25B). DAS-COL mixture was transferred to a syringe 2501 (FIG. 25C). Subsequently, another syringe 2503 was filled with a composition comprising NaOH (0.75˜2N), HEPES (200 mM) and NaHCO₃(2.2 w/v %) (FIG. 25D). Both syringes 2501 and 2503 were connected with a connector 2502 to facilitate mixing (FIG. 25E). FIG. 25F shows crosslinked DAS-COL or DAS-COL-STARCH after neutralization.

### Example 20: Comparison Between Dialdehyde Starch Crosslinked Collagen and Neutralized Collagen

Dialdehyde starch crosslinked collagen and neutralized collagen were formed using methods described hereinbefore. FIG. 26A shows examples of dialdehyde starch crosslinked collagen (3 w/v %) with DAS (0.25 w/v %). FIG. 26B shows neutralized collagen (3 w/v %). The starch crosslinked collagen showed capability to form more stable three dimensional (3d) structures. The neutralized collagen was more relaxed in form. DAS-COL can have a higher viscosity than COL (without DAS). DAS-COL is shown to be easier to print and holds its structure when printing multiple layers.

### Example 21: DAS-COL Mixed by Different Collagen and Dialdehyde Starch Concentrations

A composition that can be used as bio ink for printing was formed by mixing 3 w/v % collagen and 0.1 w/v % dialdehyde starch (FIG. 27A). FIG. 27B shows an example of bio ink for printing comprising 4 w/v % collagen and 0.1 w/v % dialdehyde starch. FIG. 27C shows disc-shaped hydrogels, which in this example comprised 4 w/v % collagen and higher dialdehyde starch of 0.25 w/v % (FIG. 27C, top row), 0.5 w/v % (FIG. 27C, middle row), or 1.0 w/v % (FIG. 27C, bottom row). FIG. 27D shows examples of lyophilized sponges comprising 4 w/v % collagen and 1.0 w/v % dialdehyde starch.

### Example 22: Printable DAS-COL with Added Starch

A 3 w/v % collagen solution, 0.25 w/v % DAS solution, and 15 w/v % starch solution were mixed the ratio of 8:1:1. FIG. 28A shows an example of a printed DAS-COL hydrogel mixed with 10% volume of starch (15 w/v % concentration). FIG. 28B shows an example of a printed DAS-COL hydrogel mixed with 20% volume of starch (15 w/v % concentration). FIG. 28C-D show examples of cultured gel in the PBS at Day +1. FIG. 28C shows the sample in FIG. 28A after culture for 1 day. FIG. 28D shows the sample in FIG. 28B after culture for 1 day. FIGS. 28E-F show examples of cultured gel in the PBS at Day +7. FIG. 28E shows the sample in FIG. 28A after culture for 7 days. FIG. 28F shows the sample in FIG. 28B after culture for 7 days.

### Example 23: A DAS-COL Degradation Test

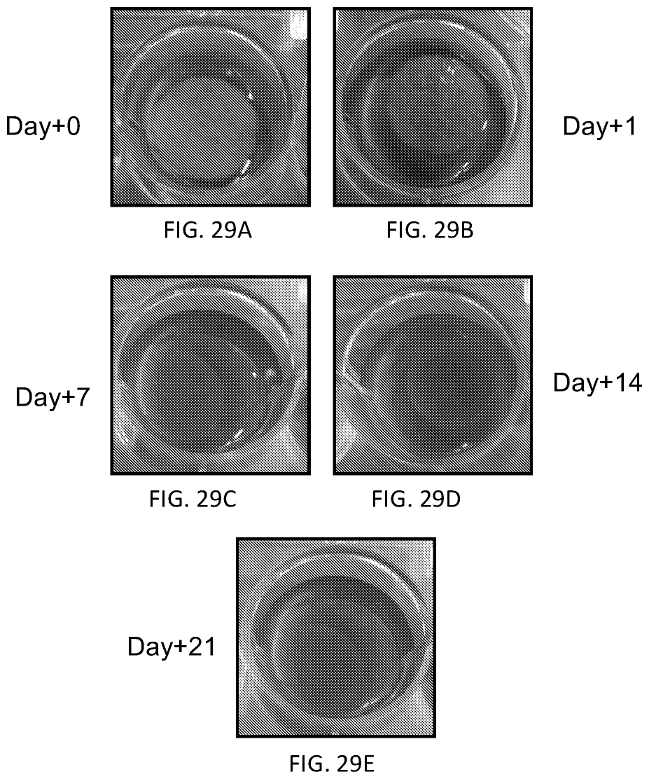

DAS-COL gels were made of 10 w/v % DAS and 4 w/v % collagen, mixing ratio of 1:9. Cross-linked DAS-COL gels were blocked with 0.75N NaOH buffer and were incubated in a 12-well plate with Dulbecco's Modified Eagle Medium (DMEM) media comprising 10% calf serum (CS) and 1% Penicillin-streptomycin-L-glutamine (PSG)) at 37° C. for 21 days. Gel degradation in culture media was observed at Day +0 (FIG. 29A); Day +1 (FIG. 29B); Day +7 (FIG. 29C); Day +14 (FIG. 29D); Day +21 (FIG. 29E).

### Example 24: Cell Viability in DAS-COL Based Gels

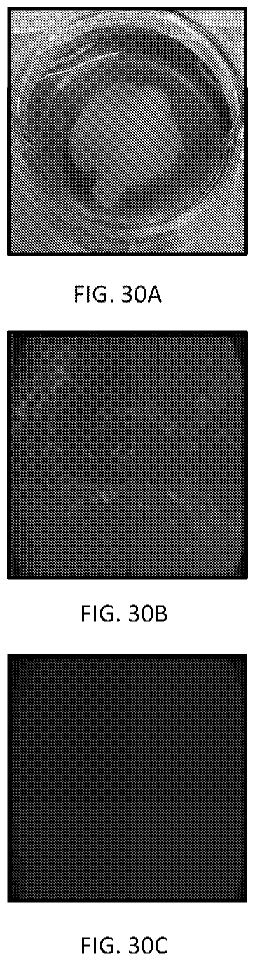

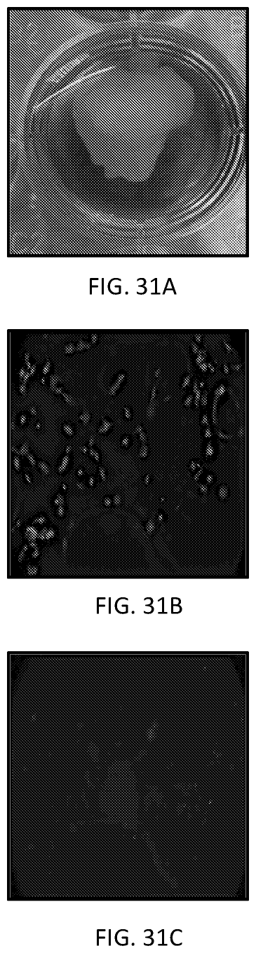

DAS-COL extruded gel (FIG. 30A) and DAS-COL-STARCH extruded gel (FIG. 31A). HUCEC cells (human umbilical vein endothelial cells) were seeded and incubated for 3 days. Cell viability was evaluated using live-dead cell fluorescent staining. Examples of a fluorescent image of live cells in DAS-COL gel is shown in FIG. 30B and in DAS-COL-STARCH is shown in FIG. 31B. Examples of a fluorescent image of dead cells in DAS-COL gel is shown in FIG. 30C and in DAS-COL-STARCH is shown in FIG. 31C. Cell viability in both gels were substantially comparable (e.g., equal cell viability).

### Example 25: Bio Printing Using DAS-COL-STARCH with Encapsulated Cells

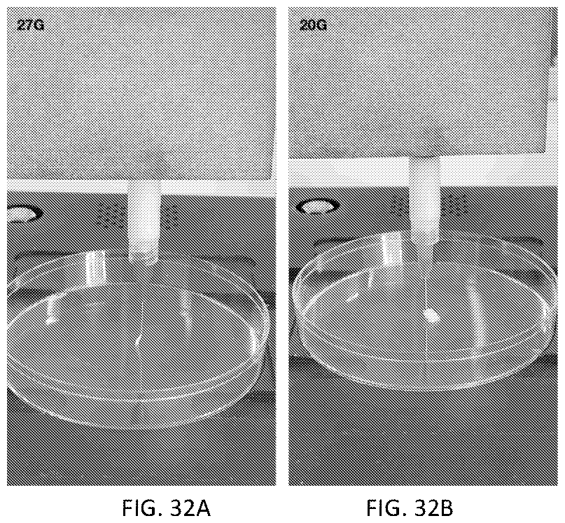

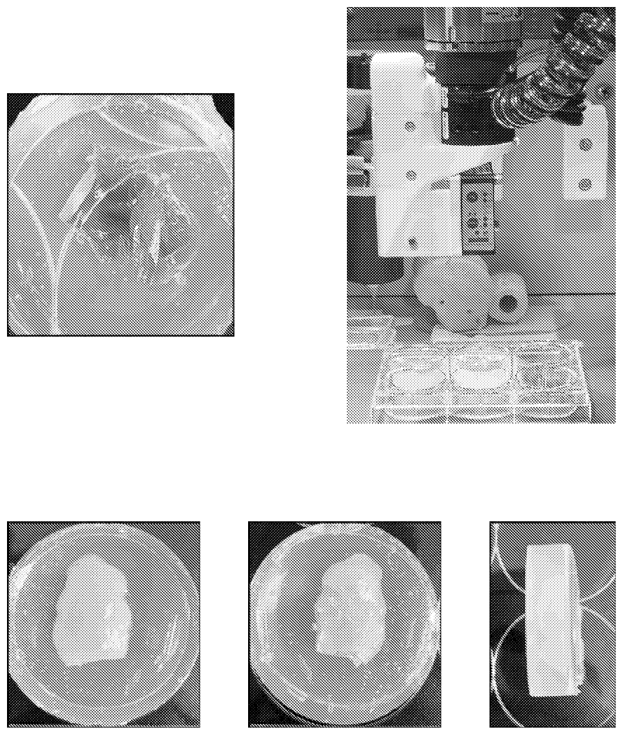

HUVEC (10 million cells/ml) were encapsulated in DAS-COL-STARCH gel, as described in example 24. The DAS-COL-STARCH gel encapsulating cells was used for bio-printing (e.g., three dimensional (3D) printing) using a 27G needle (FIG. 32A) or a 29G needle (FIG. 32B). Needles with different gauges were used to control the thickness of the bio-printing.

### Example 26: Bio Printed DAS-COL-STARCH on Articular Cartilage or Wound Defect Models

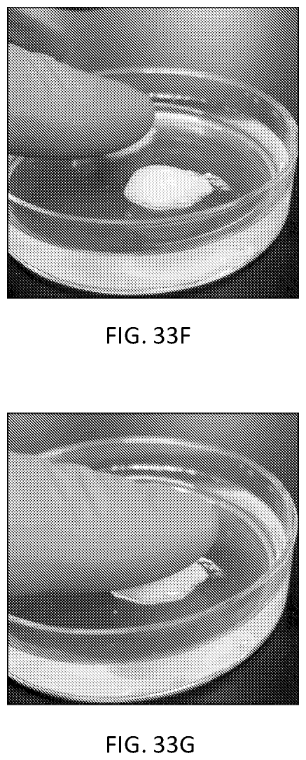

Irregular shape defects were created in agarose gel to model surgical defects (e.g., cartilage or wound defect models). An example of an irregular shape defect made in agarose in a dish is shown in FIG. 33A. DAS-COL-STARCH gel was printed in the defect using a 3D-printer. FIG. 33B shows laser scanning of the surface of the defect. The laser scanner scans the surface of the defect and surrounding area. The laser scan is used to guide the printing. After printing the laser scanner is used again to verify that the defect has been appropriately filled. FIG. 33C-33E show the gel was printed in the defect with a high accuracy from a top view, a bottom view and a side view respectively. FIG. 33F-33G show that the printed gel in this example after crosslinking was able to withstand physiologic compressive stress. FIG. 33F is printed gel on the agarose gel mold right after mixed (neutralized state) and FIG. 33G shows the same printed gel after 5 minutes on the gel mold.

### Example 27: DAS-COL Sponge In Vitro Culture and Rehydration

Lyophilized DAS-COL sponge was generated by mixing 4 w/v % collagen and 0.1 w/v % DAS at a ratio of 9:1. An example of the lyophilized DAS-COL sponge is shown in FIG. 34A. Human umbilical vein endothelial cells (HUVECs) (1 million cells/scaffold) were seeded in the lyophilized DAS-COL sponge and the scaffold was cultured in agarose gel mold for at least 3 days. FIG. 34B shows a top view and FIG. 34C shows a side view of an example of DAS-COL sponge cultured in the agarose gel mold. A section view of an example of a DAS-COL sponge after 3-day culture (FIG. 34D). The cut section DAS-COL sponge after 3-day culture was used for a cell viability assay. FIG. 34E shows an example of a DAS-COL sponge that was loaded with bone marrow or growth factor media by using a porous syringe cap system. FIG. 34F-34G show the live-dead cell staining of bone marrow cells loaded on DAS-COL sponge using a half-cut sponge. Live cells are shown in FIG. 34F and dead cells are shown in FIG. 34G.

### Example 28: Compression Test of Cylindrical DAS-COL Gel

Cylindrical DAS-COL gels were made using 4% w/v collagen and 10 w/v DAS were mixed in a volume ratio of 9:1. A compression test apparatus comprising a load cell 3501 was used to test the compression of the cylindrical DAS-COL gels. A cylindrical DAS-COL gel 3501 was placed in the compression apparatus (FIG. 35A). The compression apparatus provided an axial force to the sample to compress the sample. A height difference before and after compression was measured to calculate a percentage height change before breakage. FIG. 35B shows DAS-COL gel cylindrical block was compressed up to 50% of its initial height. The deformation was irreversible in some cases.

### Example 29: Bioprinting Parameter of DAS-COL-STARCH Encapsulated with HUVEC

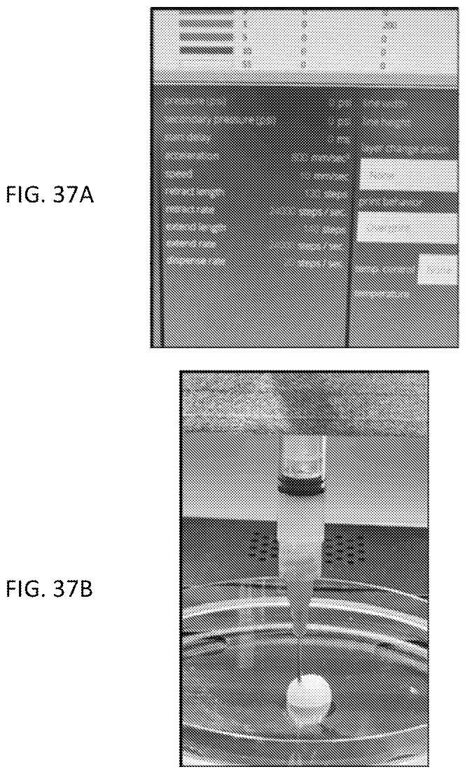

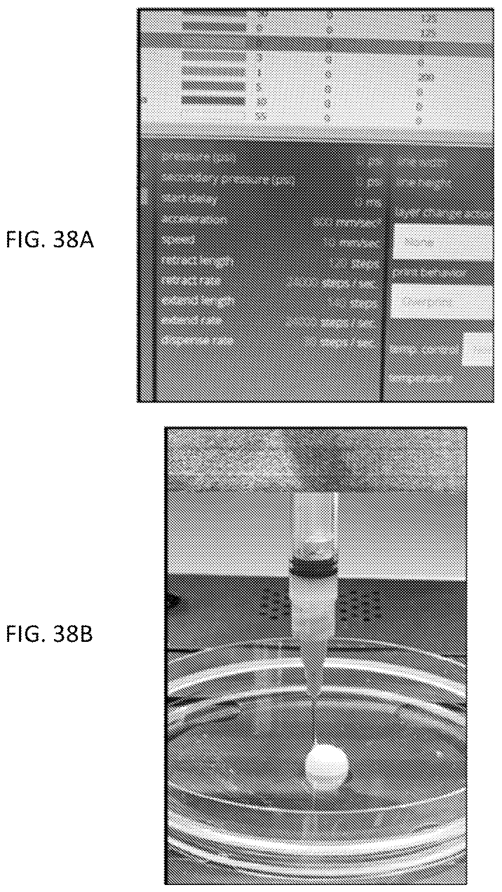

DAS-COL-STARCH gel was prepared using DAS (0.1-0.25 w/v %), COL (3 w/v %) and STARCH (15 w/v %) at a COL:DAS:STARCH of 8:1:1 ratio. HUVEC (5 million cells/ml) were encapsulated in the DAS-COL-STARCH gel and were bio-printed using a printing device. FIG. 37A and FIG. 38A show the printer parameters for two examples of bio-printing using materials described hereinbefore; the parameters comprised 10 mm/sec printing speed, retract length between 120-130 steps, 24000 steps/sec of retract rate and extend rate, 140 steps of extend length, and dispense rate between 20-30 steps/sec. FIG. 37B and FIG. 38B show examples of two three dimensional shapes that were formed using the printing process described hereinbefore.

### Example 30: HUVEC Cell Mixed DAS-COL-STARCH Gel after 7 Days

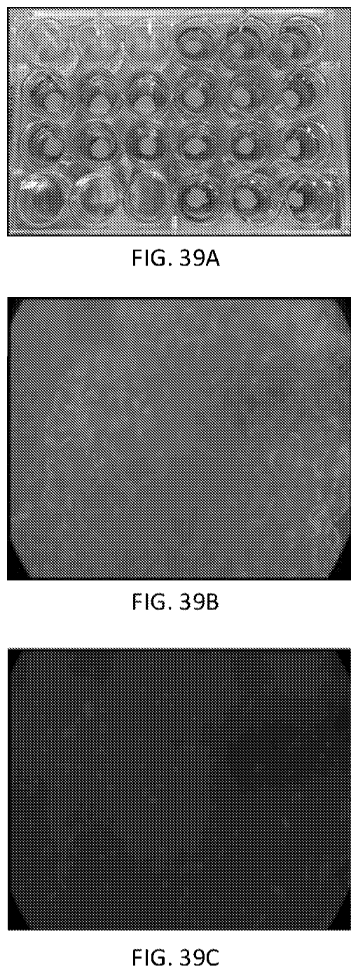

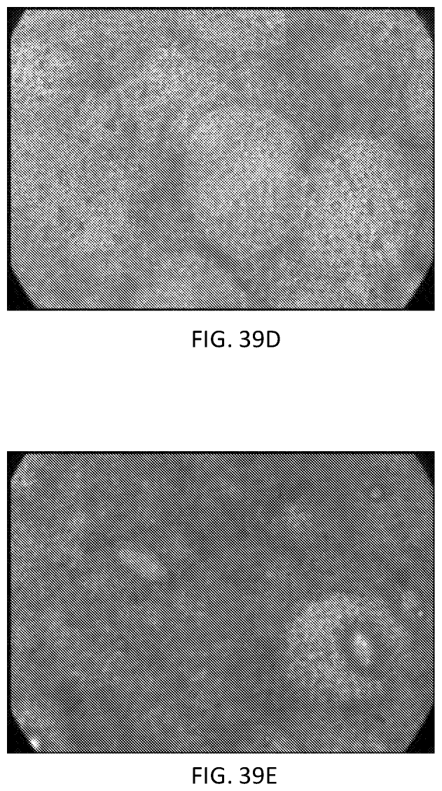

HUVEC (5M/ml) were mixed with DAS-COL-STARCH (0.25 w/v % DAS, 3 w/v % Collagen, and 15 w/v % starch in 1:8:1 ratio). Extruded gel encapsulating HUVEC cells were placed in cell growth media in a 24-well plate for 7 days (FIG. 39A). Cell viability was monitored using live-dead cell assay of HUVEC in the DAS-COL-STARCH hydrogel at day 7. An example of a fluorescent microscopy image of the live cells is shown in FIG. 39B. An example of a fluorescent microscopy image of the dead cells is shown in FIG. 39C. A majority of cells were alive at day 7 (FIG. 39B). HUVEC cells in the DAS-COL-STARCH gel in normal media or FGF (10 ng/ml) growth media were subjected to bright field microscopy. Examples of bright field microscopy images of cells are shown in FIG. 39D and FIG. 39E. FIG. 39D shows that cell density was high, and cells were fairly uniformly distributed. FIG. 39E shows that cell density significantly increased when cultured with FGF growth media.

### Example 31: Cross-Linked Human Derived Tissue and Collagen-Based Material

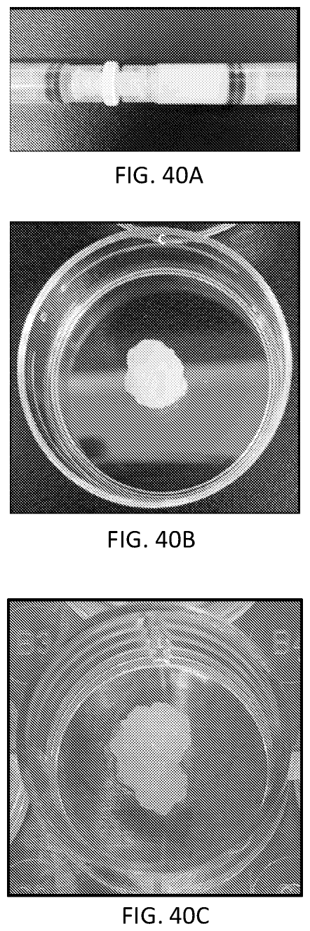

Human skin collagen type I (3 w/v %) was obtained and mixed with DAS and acetic acid. The mix was then cross-linked to form cross-linked DAS and human skin collagen type I (FIG. 40A). The crosslinked material was then extruded using an 18G syringe needle to form an arbitrary shape (FIG. 40B). FIG. 40C shows another example of crosslinking human derived tissue with DAS material. Synovial cells (25 million cells/ml) were mixed with the gel comprising the human skin collagen type I and DAS. FIG. 40C shows an example of an arbitrary shape formed from synovial cell and human skin collagen type I mix gel cross-linked with DAS. The results (FIG. 40B and FIG. 40C) showed that printed (e.g., the arbitrary shape formed using the syringe) material comprising DAS with the human skin collagen type I and/or synovial cells maintained structure.

### Example 32: Cross-Linked Human Derived ECM and Collagen-Based Material

Human extracellular matrix (ECM) extracts (1 w/v %) from placenta were obtained and mixed with DAS and acetic acid (FIG. 41A). The mix was then cross-linked to form cross-linked DAS and human ECM extracts. The crosslinked material was then extruded using an 18G syringe needle to form an arbitrary shape (FIG. 41). FIG. 41C shows another example of crosslinking human derived tissue with DAS material. Synovial cells (25 million cells/ml) were mixed with the gel comprising the ECM and DAS. FIG. 41C shows an example of an arbitrary shape formed from synovial cell mixed with human ECM extracts gel cross-linked with DAS. The results (FIG. 41B and FIG. 41C) showed that the extruded material comprising DAS, ECM and/or synovial cells maintained structure after extrusion.

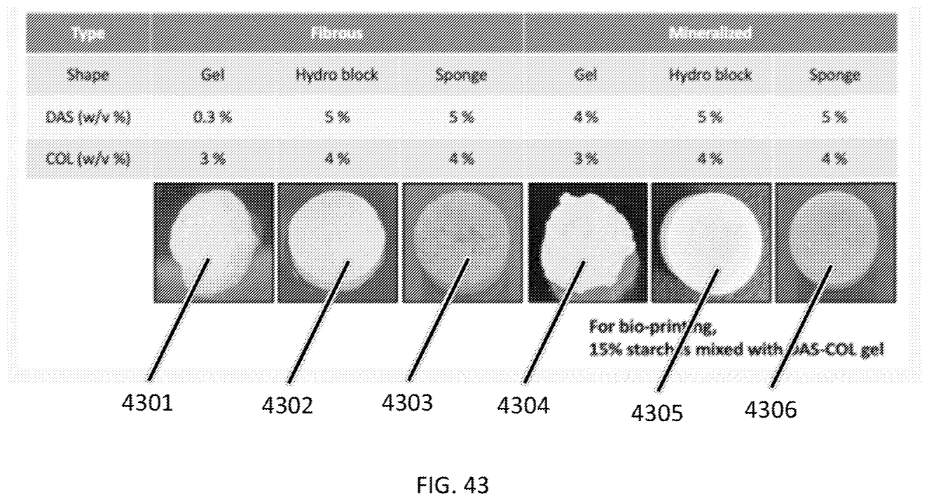

Various examples of DA-COL hydrogels made using different formulations to form into different shapes are shown in FIG. 43. A fibrous substance was formed as a gel 4301 comprising a 1:9 DAS:COL ratio of a solution comprising 0.3% w/v DAS and a solution comprising 3% w/v of COL; a hydro block 4302 comprising 5% w/v of DAS and a 4% w/v of COL at a 1:9 DAS:COL ratio; or a sponge 4303 comprising 5% w/v of DAS and a 4% w/v of COL at a 1:9 DAS:COL ratio. A fibrous substance was formed as a gel 4301 can be modified to be used for bio-printing, the modified substance can comprise a 1:8:1 volume ratio of DAS:COL:STARCH using a solution comprising 0.3% w/v DAS, a solution comprising 3% w/v of COL, and a solution comprising 15% w/v of a starch (e.g., corn starch). The fibrous sponge can be used for direct implantation of scaffold or culturing neo-tissue in cartilage defects. The fibrous gel substance is used for culturing neo-tissue or implantation in cartilage detects. The fibrous material is used along with perfusion cell culture.

A mineralized substance was formed as a gel 4304 comprising 4% w/v of DAS and a 3% w/v of COL at a 1:9 DAS:COL ratio; a hydro block 4305 comprising 5% w/v of DAS and a 4% w/v of COL at a 1:9 DAS:COL ratio; or a sponge 4306 comprising 5% w/v of DAS and a 4% w/v of COL at a 1:9 DAS:COL ratio. To generate a substance to be used for bio-printing 15% starch was mixed with the DAS-COL hydrogel. The mineralized gel can be directly printed into a defect, for example, a bone defect or be implanted as scaffold for bone defect or articular cartilage. The mineralized sponge can be implanted for scaffold in a bone defect or articular cartilage defect.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A composition, comprising:
a hydrogel comprising collagen cross-linked with dialdehyde starch; and
at least one population of cells comprising a plurality of chondrocytes, wherein the at least one population of cells is seeded on the hydrogel.

2. The composition of claim 1, wherein the collagen is Type I collagen.

3. The composition of claim 1, wherein the collagen is Type II collagen, Type IX collagen, Type X collagen, or Type XI collagen.

4. The composition of any one of claims 1-3, wherein the concentration of the collagen in the hydrogel is from 0.1% to 75% weight to volume.

5. The composition of any one of claims 1-4, wherein the concentration of the collagen in the hydrogel is from 0.5% to 50% weight to volume.

6. The composition of any one of claims 1-5, wherein the concentration of the collagen in the composition is from 4% to 8% weight to volume.

7. The composition of any one of claims 1-6, wherein the collagen is derived from an animal.

8. The composition of claim 7, wherein the collagen is derived from skin.

9. The composition of any one of claims 1-8, wherein the concentration of the dialdehyde starch in the composition is from 0.01% to 15% weight to volume.

10. The composition of any one of claims 1-9, wherein the concentration of the dialdehyde starch in the composition is from 5% to 10% weight to volume.

11. The composition of any one of claims 1-10, wherein the concentration of the dialdehyde starch in the composition is about 10% weight to volume.

12. The composition of any one of claims 1-11, wherein the composition further comprises extracellular matrix (ECM).

13. The composition of any one of claims 1-12, wherein the at least one population of cells comprises a plurality types of cells.

14. The composition of claim 13, wherein the at least one population of cells comprises at least three different types of cells, at least four different types of cells, or at least five different types of cells.

15. The composition of claim 14, wherein the at least one population of cells comprises two different types of cells, three different types of cells, four different types of cells, or five different types of cells.

16. The composition of any one of claims 1-15, wherein the at least one population of cells is randomly distributed throughout at least 10%, at least 20%, at least 30%, at least 40%, at least 50%, at least 60%, at least 70%, at least 80%, at least 90%, or 100% of the volume of the hydrogel.

17. The composition of any one of claims 1-16, wherein the at least one population of cells is evenly distributed on average throughout at least 10%, at least 20%, at least 30%, at least 40%, at least 50%, at least 60%, at least 70%, at least 80%, at least 90%, or 100% of the volume of the hydrogel.

18. The composition of any one of claims 1-17, wherein the at least one population of cells comprises from 1×10⁶ cells per mL of the hydrogel to 50×10⁶ cells per mL of the hydrogel.

19. The composition of any one of claims 1-18, wherein the at least one population of cells secretes an extracellular matrix protein.

20. The composition of any one of claims 1-19, wherein the at least one population of cells comprises at least one cell selected from the group consisting of: a synovial cell, a meniscus cell, an embryonic stem cell, a bone marrow-derived stem cell, an adipose-derived stromal cell, an infrapatellar fat pad-derived stem cell (IPFP), a pericyte, an induced pluripotent stem cell, a mesenchymal stem cell, an osteoblast, an endothelial cell, a human umbilical vein endothelial cell (HUVEC), and a myoblast.

21. The composition of any one of claims 1-20, wherein the at least one population of cells comprises at least one cell selected from the group consisting of: a human cell, a bovine cell, an equine cell, a murine cell, a canine cell, a feline cell, a lapine cell, and a porcine cell.

22. The composition of any one of claims 1-21, wherein at least 80% of cells are viable in the composition.

23. The composition of any one of claims 1-22, wherein the hydrogel is a viscous gel.

24. The composition of claim 23, wherein the viscosity of the hydrogel is from 1 to 5000 centipoise.

25. The composition of any one of claims 1-24, wherein the hydrogel has a mechanical stiffness ranging from 2 kPa to 1000 kPa.

26. The composition of any one of claims 1-24, wherein the hydrogel has a mechanical stiffness ranging from 1000 kPa to 20 GPa.

27. The composition of any one of claims 1-26, wherein the hydrogel further comprises at least one growth factor.

28. The composition of claim 27, wherein the at least one growth factor comprises a plurality of different types of growth factors.

29. The composition of claim 28, wherein the plurality of different types of growth factors comprises at least three different types of growth factors, at least four different types of growth factors, or at least five different types of growth factors.

30. The composition of claim 28, wherein the at least one growth factor comprises one type of growth factor, two different types of growth factors, three different types of growth factors, four different types of growth factors, or five different types of growth factors.

31. The composition of any one of claims 27-30, wherein the at least one growth factor is selected from the group consisting of PDGF-BB, PDGF-AA, PDGF-AB, PEDF, IGF, TGF-beta1, TGF-beta2, TGF-beta3, FGF, a BMP, EGF, GDF5, and a Wnt ligand.

32. The composition of any one of claims 1-31, further comprising heparin.

33. The composition of claim 32, wherein the heparin is conjugated to the collagen.

34. The composition of any one of claims 32-33, wherein the heparin is conjugated to a growth factor of the at least one growth factor.

35. The composition of any one of claims 1-34, wherein the hydrogel further comprises gelatin.

36. The composition of claim 31, wherein the ratio of gelatin to collagen in the hydrogel is 1:1 or less.

37. The composition of claim 35, wherein the ratio of gelatin to collagen in the hydrogel is 1:4 or less.

38. The composition of any one of claims 1-37, wherein the composition further comprises a starch.

39. The composition of claim 38, wherein a concentration of starch in the composition is from 10% w/v to 20% w/v.

40. The composition of claim 39, wherein the starch is derived from corn.

41. The composition of any one of claims 1-40, wherein the hydrogel is injectable.

42. The composition of any one of claims 1-40, wherein the hydrogel is moldable.

43. The composition of any one of claims 1-42, wherein the composition is a bio-ink composition.

44. The composition of claim 43, wherein the hydrogel is moldable into a shape selected from the group consisting of: at least a portion of a meniscus, at least a portion of an articular cartilage, at least a portion of a rib, at least a portion of an ear, at least a portion of a nose, at least a portion of a bronchial tube, at least a portion of an intervertebral disc, at least a portion of a maxillofacial cartilage, at least a portion of a temporomandibular joint (TMJ), at least a portion of a glenoid labrum, and at least a portion of an acetabular labrum, a cylinder, a cube, a three-dimensional rectangle, a sphere, a crescent, and a tetrahedron.

45. The composition of claim 43, wherein the hydrogel is printed using a three-dimensional (3D) printer.

46. The composition of claim 45, wherein the hydrogel is printed into a shape selected from the group consisting of: at least a portion of a meniscus, at least a portion of an articular cartilage, at least a portion of a rib, at least a portion of an ear, at least a portion of a nose, at least a portion of a bronchial tube, at least a portion of an intervertebral disc, at least a portion of a maxillofacial cartilage, at least a portion of a temporomandibular joint (TMJ), at least a portion of a glenoid labrum, and at least a portion of an acetabular labrum, a cylinder, a cube, a three-dimensional rectangle, a sphere, a crescent, and a tetrahedron.

47. The composition of any one of claims 1-46, wherein the hydrogel is lyophilized.

48. A layered composition comprising:
a first layer comprising a first composition according to any one of claims 1 to 47; and
a second layer comprising a second composition according to any one of claims 1 to 47 coupled to the first composition,
wherein the first composition is different than the second composition by at least one of a mechanical property, a chemical property, and a biological property.

49. The layered composition of claim 48, wherein the biological property is a type of cell in the composition, and wherein the at least one population of cells of the first layer comprises a different cell type than the at least one population of cells of the second layer.

50. The layered composition of claim 48 or 49, wherein at least 40%, at least 50%, at least 60%, at least 70%, at least 80%, at least 90%, or at least 95% of collagen molecules of the first layer are oriented at an angle of 30 degrees or less relative to a first direction.

51. The layered composition of any one of claims 48-50, wherein at least 40%, at least 50%, at least 60%, at least 70%, at least 80%, at least 90%, or at least 95% of collagen molecules of the second layer are oriented at an angle 30 degrees or less relative to a second direction.

52. The layered composition of claim 51, wherein the first direction is oriented at an angle that is at least 10 degrees, at least 20 degrees, at least 30 degrees, at least 45 degrees, or at least 60 degrees relative to the second direction.

53. The layered composition of claim 51, wherein the first direction is perpendicular to the second direction.

54. The layered composition of any one of claims 48-53, wherein the first layer is mechanically anisotropic.

55. The layered composition of any one of claims 48-54, wherein the second layer is mechanically anisotropic.

56. The layered composition of any one of claims 48-44, wherein the second layer is mechanically isotropic.

57. The layered composition of any one of claims 48-56, wherein the concentration of the collagen in the first layer is from 0.5% to 50% weight to volume.

58. The layered composition of any one of claims 48-57, wherein the concentration of the collagen in the first layer is from 4% to 8% weight to volume.

59. The layered composition of any one of claims 38-58, wherein the concentration of the dialdehyde starch in the first layer is from 0.01% to 15% weight to volume.

60. The layered composition of any one of claims 48-59, wherein the concentration of the dialdehyde starch in the first layer is from 5% to 10% weight to volume.

61. The layered composition of any one of claims 48-60, wherein the concentration of the dialdehyde starch in the first layer is 10% weight to volume.

62. The layered composition of any one of claims 48-61, wherein the concentration of the collagen in the second layer is from 0.5% to 50% weight to volume.

63. The layered composition of any one of claims 48-62, wherein the concentration of the collagen in the second layer is from 4% to 8% weight to volume.

64. The layered composition of any one of claims 48-63, wherein the concentration of the dialdehyde starch in the second layer is from 0.01% to 15% weight to volume.

65. The layered composition of any one of claims 48-64, wherein the concentration of the dialdehyde starch in the second layer is from 5% to 10% weight to volume.

66. The layered composition of any one of claims 48-65, wherein the concentration of the dialdehyde starch in the second layer is 10% weight to volume.

67. The layered composition of any one of claims 48-66, wherein the first layer comprises at least one growth factor.

68. The layered composition of any one of claims 48-67, wherein the second layer comprises at least one growth factor.

69. The layered composition of any one of claims 67-68, wherein the at least one growth factor comprises a plurality of different types of growth factors.

70. The layered composition of claim 69, wherein the plurality of different types of growth factors comprises at least three different types of growth factors, at least four different types of growth factors, or at least five different types of growth factors.

71. The layered composition of claim 69, wherein the at least one growth factor comprises two different types of growth factors, three different types of growth factors, four different types of growth factors, or five different types of growth factors.

72. The layered composition of any one of claims 67-71, wherein the at least one growth factor is selected from the group consisting of PDGF-BB, PDGF-AA, PDGF-AB, PEDF, IGF, TGF-beta1, TGF-beta2, TGF-beta3, FGF, a BMP, EGF, GDF5, and a Wnt ligand.

73. The layered composition of claim 72, wherein the ratio of gelatin to collagen in the hydrogel is 1:4 or less.

74. The layered composition of any one of claims 67-72, wherein a growth factor of the at least one growth factor is conjugated with heparin.

75. The layered composition of any one of claims 48-74, further comprising a buffer.

76. The layered composition of claim 75, wherein the buffer is a zwitterionic buffer.

77. The layered composition of claim 75 or claim 76, wherein the buffer is selected from the group consisting of: MES, ADA, PIPES, ACES, MOPSO, MOPS, BES, TES, HEPES, DIPSO, Acetamidoglycine, TAPSO, POPSO, HEPPSO, HEPPS, Tricine, Glycinamide, Bicine, and TAPS.

78. A method of forming a hydrogel, comprising:
mixing a collagen with a dialdehyde starch and a population of cells comprising a plurality of chondrocytes.

79. The method of claim 78, wherein the method further comprises mixing a starch.

80. The method of claim 79, wherein a concentration of starch in the hydrogel is from 10% w/v to 20% w/v.

81. The method of claim 80, wherein the starch is derived from corn.

82. The method of claim 78-81, wherein the collagen is Type I collagen.

83. The method of claim 78-82, wherein the collagen is Type II collagen, Type IX collagen, Type X collagen, or Type XI collagen.

84. The method of any one of claims 78-83, wherein the concentration of the collagen in the hydrogel is from 0.1% to 75% weight to volume.

85. The method of any one of claims 78-84, wherein the concentration of the collagen in the hydrogel is from 0.5% to 50% weight to volume.

86. The method of any one of claims 78-85, wherein the concentration of the collagen in the hydrogel is from 4% to 8% weight to volume.

87. The method of any one of claims 78-86, wherein the collagen is derived from an animal.

88. The method of claim 87, wherein the collagen is derived from skin.

89. The method of any one of claims 78-88, wherein the concentration of the dialdehyde starch in the hydrogel is from 0.01% to 15% weight to volume.

90. The method of any one of claims 78-89, wherein the concentration of the dialdehyde starch in the hydrogel is from 5% to 10% weight to volume.

91. The method of any one of claims 78-90, wherein the concentration of the dialdehyde starch in the hydrogel is about 10% weight to volume.

92. The method of any one of claims 78-91, wherein the method further comprises adding extracellular matrix (ECM) to the mixing.

93. The method of any one of claims 78-92, wherein the at least one population of cells comprises a plurality types of cells.

94. The method of claim 93, wherein the at least one population of cells comprises at least three different types of cells, at least four different types of cells, or at least five different types of cells.

95. The method of claim 93, wherein the at least one population of cells comprises two different types of cells, three different types of cells, four different types of cells, or five different types of cells.

96. The method of any one of claims 78-95, wherein the at least one population of cells is randomly distributed throughout at least 10%, at least 20%, at least 30%, at least 40%, at least 50%, at least 60%, at least 70%, at least 80%, at least 90%, or 100% of the volume of the hydrogel.

97. The method of any one of claims 78-96, wherein the at least one population of cells is evenly distributed on average throughout at least 10%, at least 20%, at least 30%, at least 40%, at least 50%, at least 60%, at least 70%, at least 80%, at least 90%, or 100% of the volume of the hydrogel.

98. The method of any one of claims 78-97, wherein the at least one population of cells comprises from 1×10⁶ cells per mL of the hydrogel to 50×10⁶ cells per mL of the hydrogel.

99. The method of any one of claims 78-98, wherein the at least one population of cells comprises at least one cell selected from the group consisting of: a synovial cell, a meniscus cell, an embryonic stem cell, a bone marrow-derived stem cell, an adipose-derived stromal cell, an infrapatellar fat pad-derived stem cell (IPFP), a pericyte, an induced pluripotent stem cell, a mesenchymal stem cell, an osteoblast, an endothelial cell, a human umbilical vein endothelial cell (HUVEC), and a myoblast.

100. The method of any one of claims 78-99, wherein the at least one population of cells comprises at least one cell selected from the group consisting of: a human cell, a bovine cell, an equine cell, a murine cell, a canine cell, a feline cell, a lapine cell, and a porcine cell.

101. The method of any one of claims 78-100, wherein the at least one population of cells secretes an extracellular matrix protein.

102. The method of any one of claims 78-101, wherein the mixing comprises steps of:
generating a first mixture of collagen and at least one population of cells comprising a plurality of chondrocytes;
generating the hydrogel by adding dialdehyde starch to the first mixture.

103. The method of any one of claims 78-102, further comprising neutralizing a pH of the hydrogel while the collagen, the population of cells, and the dialdehyde starch are mixed.

104. The method of claim 103, wherein the neutralizing comprises adding a buffer.

105. The method of claim 104, wherein the buffer is zwitterionic.

106. The method of claim 104 or 105, wherein the buffer is selected from the group consisting of: MES, ADA, PIPES, ACES, MOPSO, MOPS, BES, TES, HEPES, DIPSO, Acetamidoglycine, TAPSO, POPSO, HEPPSO, HEPPS, Tricine, Glycinamide, Bicine, and TAPS.

107. The method of any one of claims 78-106, wherein the hydrogel is a viscous gel.

108. The method of claim 107, wherein the viscosity of the hydrogel is from 1 to 5000 centipoise.

109. The method of any one of claims 78-108, wherein the hydrogel has a mechanical stiffness ranging from 2 kPa to 1000 kPa.

110. The method of any one of claims 78-108, wherein the hydrogel has a mechanical stiffness ranging from 1000 kPa to 20 GPa.

111. The method of any one of claims 78-110, wherein the hydrogel further comprises at least one growth factor.

112. The method of claim 111, wherein the at least one growth factor comprises a plurality of different types of growth factors.

113. The method of claim 112, wherein the plurality of different types of growth factors comprises at least three different types of growth factors, at least four different types of growth factors, or at least five different types of growth factors.

114. The method of claim 112, wherein the at least one growth factor comprises two different types of growth factors, three different types of growth factors, four different types of growth factors, or five different types of growth factors.

115. The method of any one of claims 94-114, wherein the at least one growth factor is selected from the group consisting of PDGF-BB, PDGF-AA, PDGF-AB, PEDF, IGF, TGF-beta1, TGF-beta2, TGF-beta3, FGF, a BMP, EGF, GDF5, and a Wnt ligand.

116. The method of any one of claims 78-115, further comprising heparin.

117. The method of claim 116, wherein the heparin is conjugated to the collagen.

118. The method of any one of claims 99-117, wherein the heparin is conjugated to a growth factor of the at least one growth factor.

119. The method of any one of claims 78-118, wherein the hydrogel further comprises gelatin.

120. The method of claim 118, wherein the ratio of gelatin to collagen in the hydrogel is 1:1 or less.

121. The method of claim 118, wherein the ratio of gelatin to collagen in the hydrogel is 1:4 or less.

122. The method of any one of claims 78-103, wherein the collagen, the dialdehyde starch, and the population of cells are mixed in a mold.

123. The method of claim 122, wherein a shape of the mold comprises at least a portion of a meniscus, at least a portion of an articular cartilage, at least a portion of a rib, at least a portion of an ear, at least a portion of a nose, at least a portion of a bronchial tube, at least a portion of an intervertebral disc, at least a portion of a maxillofacial cartilage, at least a portion of a temporomandibular joint (TMJ), at least a portion of a glenoid labrum, at least a portion of an acetabular labrum, a cylinder, or any combinations thereof.

124. The method of any one of claims 78-123, further comprising extruding a pattern onto a substrate with the collagen, the dialdehyde starch, and the population of cells as they are mixed.

125. The method of any one of claims 78-123, further comprising depositing the collagen, the dialdehyde starch, and the population of cells as they are mixed in a predetermined shape onto a substrate.

126. The method of any one of claims 78-125, wherein at least 80% of the populations of cells are viable.

127. The method of claim 126, wherein the substrate comprises a target region of a subject.

128. The method of claim 127, wherein the target region comprises at least a portion of a knee joint, at least a portion of a shoulder joint, at least a portion of a hip joint, at least a portion of a temporomandibular joint (TMJ), at least a portion of a rib, at least a portion of an ear, at least a portion of a nose, at least a portion of a bronchial tube, at least a portion of an intervertebral disc, or at least a portion of a maxillofacial cartilage.

129. The method of claim 127, wherein the at least a portion of a knee joint comprises a meniscus.

130. The method of claim 127, wherein the at least a portion of a shoulder joint comprises a glenoid labrum.

131. The method of claim 127, wherein the at least a portion of a hip joint comprises an acetabular labrum.

132. The method of claim 127, wherein the at least a portion of a temporomandibular joint comprises a maxillofacial cartilage.

133. The method of any one of claims 125-132, wherein the predetermined shape is at least 50%, at least 60%, at least 70%, at least 80%, or at least 90% identical to the target region.

134. A method of forming a hydrogel scaffold, comprising:
mixing a collagen with a dialdehyde starch to cross-link the collagen into a hydrogel; and
seeding the cross-linked collagen with at least one population of cells comprising a plurality of chondrocytes.

135. The method of claim 134, wherein the method further comprises mixing a starch.

136. The method of claim 135, wherein a concentration of starch in the hydrogel scaffold is from 10% w/v to 20% w/v.

137. The method of claim 136, wherein the starch is derived from corn.

138. The method of claim 134-137, wherein the collagen is Type I collagen.

139. The method of claim 134-137, wherein the collagen is Type II collagen, Type IX collagen, Type X collagen, or Type XI collagen.

140. The method of any one of claims 134-139, wherein the concentration of the collagen in the hydrogel scaffold is from 0.1% to 75% weight to volume.

141. The method of any one of claims 134-140, wherein the concentration of the collagen in the hydrogel scaffold is from 0.5% to 50% weight to volume.

142. The method of any one of claims 134-141, wherein the concentration of the collagen in the hydrogel scaffold is from 4% to 8% weight to volume.

143. The method of any one of claims 134-142, wherein the collagen is derived from an animal.

144. The method of claim 143, wherein the collagen is derived from skin.

145. The method of any one of claims 134-144, wherein the concentration of the dialdehyde starch in the hydrogel scaffold is from 0.01% to 15% weight to volume.

146. The method of any one of claims 134-145, wherein the concentration of the dialdehyde starch in the hydrogel scaffold is from 5% to 10% weight to volume.

147. The method of any one of claims 134-146, wherein the concentration of the dialdehyde starch in the hydrogel scaffold is about 10% weight to volume.

148. The method of any one of claims 119-127, wherein the method further comprises mixing extracellular matrix (ECM).

149. The method of any one of claims 134-148, wherein the at least one population of cells comprises a plurality types of cells.

150. The method of claim 149, wherein the at least one population of cells comprises at least three different types of cells, at least four different types of cells, or at least five different types of cells.

151. The method of claim 149, wherein the at least one population of cells comprises two different types of cells, three different types of cells, four different types of cells, or five different types of cells.

152. The method of any one of claims 134-151, wherein the at least one population of cells is randomly seeded throughout at least 10%, at least 20%, at least 30%, at least 40%, at least 50%, at least 60%, at least 70%, at least 80%, at least 90%, or 100% of the volume of the hydrogel scaffold.

153. The method of any one of claims 134-152, wherein the at least one population of cells is evenly seeded on average throughout at least 10%, at least 20%, at least 30%, at least 40%, at least 50%, at least 60%, at least 70%, at least 80%, at least 90%, or 100% of the volume of the hydrogel scaffold.

154. The method of any one of claims 134-153, wherein the at least one population of cells is seeded in a pattern within at least 10%, at least 20%, at least 30%, at least 40%, at least 50%, at least 60%, at least 70%, at least 80%, at least 90%, or 100% of the volume of the hydrogel scaffold.

155. The method of any one of claims 134-154, wherein the at least one population of cells comprises from 1×10⁶ cells per mL of the hydrogel scaffold to 50×10⁶ cells per mL of the hydrogel scaffold.

156. The method of any one of claims 134-155, wherein the at least one population of cells comprises at least one cell selected from the group consisting of: a synovial cell, a meniscus cell, an embryonic stem cell, a bone marrow-derived stem cell, an adipose-derived stromal cell, an infrapatellar fat pad-derived stem cell (IPFP), a pericyte, an induced pluripotent stem cell, a mesenchymal stem cell, an osteoblast, an endothelial cell, a human umbilical vein endothelial cell (HUVEC), and a myoblast.

157. The method of any one of claims 134-156, wherein the at least one population of cells comprises at least one cell selected from the group consisting of: a human cell, a bovine cell, an equine cell, a murine cell, a canine cell, a feline cell, a lapine cell, and a porcine cell.

158. The method of any one of claims 134-157, wherein the at least one population of cells secretes an extracellular matrix protein.

159. The method of any one of claims 134-158, further comprising neutralizing a pH of the hydrogel before seeding the cross-linked collagen with the at least one population of cells.

160. The method of claim 159, wherein the neutralizing comprises adding a buffer.

161. The method of claim 160, wherein the buffer is zwitterionic.

162. The method of claim 160 or 161, wherein the buffer is selected from the group consisting of: MES, ADA, PIPES, ACES, MOPSO, MOPS, BES, TES, HEPES, DIPSO, Acetamidoglycine, TAPSO, POPSO, HEPPSO, HEPPS, Tricine, Glycinamide, Bicine, and TAPS.

163. The method of any one of claims 134-162, further comprising placing the hydrogel into a mold.

164. The method of any one of claims 134-163, further comprising placing the hydrogel scaffold into a mold.

165. The method of claim 163 or 164, wherein a shape of the mold comprises at least a portion of a meniscus, at least a portion of an articular cartilage, at least a portion of a rib, at least a portion of an ear, at least a portion of a nose, at least a portion of a bronchial tube, at least a portion of an intervertebral disc, at least a portion of a maxillofacial cartilage, at least a portion of a temporomandibular joint (TMJ), at least a portion of a glenoid labrum, at least a portion of an acetabular labrum, a cylinder, or any combinations thereof.

166. The method of any one of claims 134-165, wherein the collagen is conjugated with a heparin.

167. The method of claim 166, wherein the heparin is conjugated with at least one growth factor.

168. The method of claim 167, wherein at least two different growth factors, at least three different growth factors, at least four different growth factors, or at least five different growth factors are conjugated to the heparin.

169. The method of claim 167, wherein two different growth factors, three different growth factors, four different growth factors, or five different growth factors are conjugated to the heparin.

170. The method of any one of claims 167-169, wherein the growth factor is selected from the group consisting of PDGF-BB, PDGF-AA, PDGF-AB, PEDF, IGF, TGF-beta1, TGF-beta2, TGF-beta3, FGF, a BMP, EGF, GDF5, and a Wnt ligand.

171. The method of any one of claims 134-170, wherein the hydrogel further comprises gelatin.

172. The method of claim 171, wherein the ratio of gelatin to collagen in the hydrogel is 1:1 or less.

173. The method of any one of claims 134-172, wherein the hydrogel is a viscous gel.

174. The method of any one of claims 134-173, wherein the viscosity of the hydrogel is from 1 to 5000 centipoise.

175. The method of any one of claims 134-174, wherein the hydrogel is injectable.

176. The method of any one of claims 134-175, wherein the hydrogel has a mechanical stiffness ranging from 2 kPa to 1000 kPa.

177. The method of any one of claims 134-175, wherein the hydrogel has a mechanical stiffness ranging from 1000 kPa to 20 GPa.

178. A method comprising forming a layered hydrogel by forming a first layer comprising a hydrogel according to any one of claims 78-177 and a second layer comprising a composition according to any one of claims 78-177, wherein the composition of the first layer is different than the composition of the second layer by at least one of a mechanical property, a chemical property, and a biological property.

179. The method of claim 178, wherein the biological property is a type of cell in the composition, and wherein the at least one population of cells of the first layer comprises a different cell type than the at least one population of cells of the second layer.

180. The method of claim 178 or 179, wherein at least 40%, at least 50%, at least 60%, at least 70%, at least 80%, at least 90%, or at least 95% of collagen molecules of the first layer are oriented at an angle of 30 degrees or less relative to a first direction.

181. The method of any one of claims 178-180, wherein at least 40%, at least 50%, at least 60%, at least 70%, at least 80%, at least 90%, or at least 95% of collagen molecules of the second layer are oriented at an angle 30 degrees or less relative to a second direction.

182. The method of claim 181, wherein the first direction is oriented at an angle that is at least 10 degrees, at least 20 degrees, at least 30 degrees, at least 45 degrees, or at least 60 degrees relative to the second direction.

183. The method of claim 182, wherein the first direction is perpendicular to the second direction.

184. The method of any one of claims 178-183, wherein the second layer is mechanically anisotropic.

185. The method of any one of claims 154-184, wherein the second layer is mechanically isotropic.

186. The method of any one of claims 178-185, wherein the concentration of the collagen in the first layer is from 0.5% to 50% weight to volume.

187. The method of any one of claims 178-186, wherein the concentration of the collagen in the first layer is from 4% to 8% weight to volume.

188. The method of any one of claims 178-187, wherein the concentration of the dialdehyde starch in the first layer is from 0.01% to 15% weight to volume.

189. The method of any one of claims 178-188, wherein the concentration of the dialdehyde starch in the first layer is from 5% to 10% weight to volume.

190. The method of any one of claims 178-189, wherein the concentration of the dialdehyde starch in the first layer is 10% weight to volume.

191. The method of any one of claims 178-190, wherein the concentration of the collagen in the second layer is from 0.5% to 50% weight to volume.

192. The method of any one of claims 178-191, wherein the concentration of the collagen in the second layer is from 4% to 8% weight to volume.

193. The method of any one of claims 178-192, wherein the concentration of the dialdehyde starch in the second layer is from 0.01% to 15% weight to volume.

194. The method of any one of claims 178-193, wherein the concentration of the dialdehyde starch in the second layer is from 5% to 10% weight to volume.

195. The method of any one of claims 178-194, wherein the concentration of the dialdehyde starch in the second layer is 10% weight to volume.

196. The method of any one of claims 178-195, wherein the first layer comprises at least one growth factor.

197. The method of any one of claims 178-196, wherein the second layer comprises at least one growth factor.

198. The method of any one of claims 178-197, wherein the at least one growth factor comprises a plurality of different types of growth factors.

199. The method of claim 198, wherein the plurality of different types of growth factors comprises at least three different types of growth factors, at least four different types of growth factors, or at least five different types of growth factors.

200. The method of claim 199, wherein the at least one growth factor comprises two different types of growth factors, three different types of growth factors, four different types of growth factors, or five different types of growth factors.

201. The method of any one of claims 197-200, wherein the at least one growth factor is selected from the group consisting of PDGF-BB, PDGF-AA, PDGF-AB, PEDF, IGF, TGF-beta1, TGF-beta2, TGF-beta3, FGF, a BMP, EGF, GDF5, and a Wnt ligand.

202. The method of any one of claims 197-201, wherein a growth factor of the at least one growth factor is conjugated with heparin.

203. The method of any one of claims 178-202, further comprising a buffer.

204. The method of claim 203, wherein the buffer is a zwitterionic buffer.

205. The method of claim 203 or 204, wherein the buffer is selected from the group consisting of: MES, ADA, PIPES, ACES, MOPSO, MOPS, BES, TES, HEPES, DIPSO, Acetamidoglycine, TAPSO, POPSO, HEPPSO, HEPPS, Tricine, Glycinamide, Bicine, and TAPS.

206. The method of any one of claims 178-205, wherein the first and second layers are in different shapes.

207. The method of any one of claims 178-206, wherein the first and second layers are in different pH.

208. The method of any one of claims 178-207, wherein the first and second layers have different stiffness.
